(19) United States
(12) Patent Application Publication
(10) Pub. No.: US 2024/0309301 A1
(43) Pub. Date: Sep. 19, 2024

(54) Biobased Core-Shell Microcapsules

(71) Applicant: Symrise AG, Holzminden (DE)

(72) Inventors: John Teffenhart, Edison, NJ (US); Katherine Tecchio, Parsippany, NJ (US); Rose Scott, Queens Village, NY (US); Jean Eisenhauer, Staten Island, NY (US)

(21) Appl. No.: 18/577,687

(22) PCT Filed: Dec. 21, 2021
(86) PCT No.: PCT/EP2021/086989

§ 371 Date: Jan. 8, 2024

Related U.S. Application Data
(60) Provisional application No. 63/227,700, filed on Jul. 30, 2021.

(51) Int. Cl. C11D 17/00; B01J 13/16; C11D 3/00; C11D 3/37; C11D 3/50

(52) U.S. Cl. CPC C11D 17/0039; B01J 13/16; C11D 3/001; C11D 3/3726; C11D 3/505

(57) ABSTRACT

The present invention relates to (amino)saccharide-based polyurea and/or polyurethane-based microcapsules containing at least one lipophilic active ingredient, which have a good balance of increased biodegradability, stability in product formulations, and performance compared to commercially available state-of-the-art microcapsules, which are fully synthetically based. Moreover, the present invention discloses a microcapsule slurry comprising a plurality of said core-shell microcapsules dispersed in an aqueous phase. In addition, the present invention relates to a process for the preparation of a microcapsule slurry and the biobased core-shell microcapsules contained therein as such. In a further aspect, the invention described herein relates to the use of such microcapsules or microcapsule dispersions comprising the microcapsules according to the invention for the production of various consumer products. Finally, the present invention also relates to consumer products comprising such microcapsules or microcapsule dispersions.

### FIELD OF THE INVENTION

The present invention relates to biobased core-shell microcapsules that contain natural (amino)saccharide segments in the capsule wall as building blocks making the shell wall of the biobased microcapsules according to the invention more biodegradable than the existing fully synthetically created state-of-the-art microcapsules being based on non-biobased segments. More specifically, the present invention relates to biobased microcapsules comprising a microcapsule shell being based on polyurea and/or polyurethane structures formed using monomeric or short-chained saccharides and/or aminosaccharides as building blocks and containing at least one lipophilic active ingredient, which have a good balance of increased biodegradability, stability in product formulations and performance (targeted release properties) compared to commercially available state-of-the-art microcapsules. Moreover, the present invention discloses a slurry comprising a plurality of said biobased core-shell microcapsules dispersed in an aqueous phase (microcapsule dispersion). In addition, the present invention relates to a process for the preparation of a slurry and the biobased core-shell microcapsules contained therein as such. In a further aspect, the invention described herein relates to the use of such microcapsules or microcapsule dispersions comprising the microcapsules according to the invention for the preparation of various consumer products. Finally, the present invention also relates to these consumer products comprising said microcapsules or microcapsule dispersions.

### PRIOR ART

Nowadays, for example, many consumer products such as detergents, fabric softeners, washing powders, liquid detergents, shower gels, shampoos, deodorants, lotions, etc. are perfumed with fragrance materials or contain cosmetic ingredients to deliver a specific effect. Unfortunately, often, for example, the fragrant substances or cosmetic ingredients of such products interact with other components of the product formulation, or the more volatile components of the perfume formulation tend to evaporate prematurely. Consequently, this leads to an undesired change and/or decrease of the fragrance impression or premature “use” of the cosmetic ingredient over time or might even cause disadvantageous reactions with the other ingredients of the product formulation resulting in a reduced product quality and/or stability. In order to prevent possible interactions of the fragrances or other active components with the other ingredients of the product or, for example, to prevent the volatilization of the fragrances and thus not to distort or reduce the desired olfactory impression, the fragrances or other active ingredients can be added to the formulation in encapsulated form. In this way, for example the desired olfactory impression can be guaranteed. Moreover, interactions between the product components can be reduced allowing for improved product quality and storage stability.

Encapsulation is a common technique used to protect active components (also known as actives or active ingredients) and to release said substances forming the core of the capsule in a targeted manner at a specific point in time. In addition, some active ingredients or active components can often not be used directly for various reasons (e.g., due to their solubility, reactivity, stability, etc.). However, based on the encapsulation method and the chosen materials, lipophilic or hydrophobic active ingredients, such as fragrances, cosmetic ingredients, or flavorings, can be easily and stably incorporated into various product formulations thus reducing or completely preventing interactions in the perfumed product or the evaporation of the highly volatile fragrance components.

The contents of such core-shell microcapsules can be released in various ways, for example by mechanical destruction of the capsule wall by crushing or shearing, destruction of the capsule by melting the wall material, destruction of the capsules by dissolving the wall material or diffusion of the active ingredients through the capsule wall after activation. Alternatively, the shell wall could be destroyed by biological or enzymatic interactions to release the active ingredient(s) in a targeted manner.

Interfacial polymerization is an efficient and common encapsulation process for the production of core-shell microcapsules, whereby a suspension is produced starting from an initial emulsion or suspension of the hydrophobic core material, i.e., the active component, in water. The monomers or reagents in the aqueous phase diffuse through the interface to react with the reactants of the oil phase or monomers of the aqueous phase react with themselves and are deposited in a layer around the emulsion droplets or the dispersed liquid particles of the core material and polymerize to form a compact crosslinked and continuous capsule shell. Depending on the wall material and degree of crosslinking, different properties of the microcapsules can be achieved.

Polyurea-based, polyurethane-based or polyurea/polyurethane mixed microcapsules, which are formed by interfacial polymerization between a polyisocyanate dissolved in the hydrophobic phase and a polyamine and/or a diol or polyol dissolved in the aqueous phase, are well-known capsules used in a variety of technical fields, including perfumery.

Many currently developed polyurea and/or polyurethane-based encapsulation formulations involve the use of fragrances dissolved with at least one polyisocyanate dispersed in an aqueous phase containing at least one polyfunctional amine (such as guanidine salts, aromatic, aliphatic or alkyl polyamines) and/or at least one polyol (such as glycerol or alkyl, alkylene oxide, aliphatic and aromatic polyols) with emulsion stabilizing agents (such as surfactants or protective colloids). Alternatively, the liquid cores of the emulsion are stabilized by using solid particles being smaller than 1 μm in size (such as starches, silicas, metal oxides) instead of protective colloids and/or surfactants which adsorb onto the interface between the two liquid phases (so-called Pickering emulsions).

By diffusion of small monomers, i.e., the shell building blocks, the shell is initially rapidly forming. However, as the shell grows, the diffusion rate of the monomers through the shell towards the oil phase decreases and the rate of the shell formation reaction drops. In the case of larger monomers as building blocks, on the other hand, diffusion into the reaction area (i.e., the oil phase) is hindered and thus generally low and therefore only surface crosslinked structures are formed resulting in a reduced reaction of said larger building blocks with the polyisocyanate component, causing the active ingredient to diffuse out of the capsule prior to the targeted release due to the high porosity of the capsule wall in combination with its reduced mechanical stability. In addition, these formulations for the preparation of oil core-shell microparticles require the use of protective colloids such as polyvinyl alcohols (PVA), polyvinylpyrrolidones, water-soluble acrylate copolymers, modified starches, gelatin, gum arabic, cellulosics such as carboxymethycellulose, and other large polysaccharides, and/or surface active nonionic, cationic or anionic substances (so-called surfactants) as formation aids, which arrange at the surface of the oil droplets of the emulsion to lower the surface tension of the aqueous phase. Thereby, the interfacial polymerization reaction of the polyisocyanates in the oil phase and the polyamine and/or polyol in the aqueous phase on the surface of the oil droplets is limited due to the low diffusion extent of the building blocks towards the interface of the oil phase and the aqueous phase as the diffusion is hindered, thus likewise resulting in surface crosslinked structures with a limited degree of urea/urethane-based linkages.

However, the state-of-the-art core-shell microcapsules have the disadvantage that their fully synthetic shell materials are largely hydrolytically stable and resistant to environmental decomposition.

Because non-biodegradable plastic particles are increasingly subject to public criticism with regard to their environmental impact, and because the demand for biobased and more biodegradable solutions is growing due to the ever-increasing social pressure and new and future regulatory (governmental, country and regional) regulations regarding plastics in terms of environmental and health aspects, there is a need for the development of new encapsulation materials in order to achieve a reduction of microplastics in the environment and an increase of biodegradability towards more eco-sensitive and preferably biobased products. Thus, nowadays, primarily biobased and biodegradable materials are the focus of attention.

The state-of-the-art microcapsules, produced by interfacial polymerization are either based on fully synthetically derived polyurea, polyurethane, aminoplast, polyacrylate structures or mixtures of these structures that are largely non-biodegradable in the environment (by having a direct negative environmental impact or by resulting in non-degradable residues) or do not exhibit sufficient stability for the efficient encapsulation of active ingredients. Therefore, microcapsules that are not solely synthetic and biodegradable in the environment and which are simultaneously highly stable are required.

First steps towards more natural-based products have been made by preparing biodegradable coatings on non-degradable core-shell capsule substrates called “green washing” to achieve the impression of “biodegradation”, for example by using high molecular weight polysaccharides and amino-polysaccharides such as high molecular weight maltodextrins and chitosan as additional coatings on polyurea-based core-shell substrates instead of substances such as polyaldehydes and polyvalent salts for additional stability of the microcapsules. However, since chitosan is only soluble at a few percent in acidic solutions, its direct incorporation into the shell of the microparticles is strongly limited. Furthermore, attempts were made to partially incorporate biodegradable starches and celluloses into synthetic polymers to achieve the impression of increased “biodegradability”.

For example, WO 2020/058044 A1 (Givaudan) describes a consumer product comprising a plurality of microcapsules dispersed in a dispersing medium, wherein the shells of the microcapsules are coated with chitosan and wherein the dispersing medium comprises additional free chitosan. Thereby, the free chitosan comprised in the dispersing medium has preferably a molecular weight between 500 000 g/mol and 3 000 000 g/mol and the chitosan that is coating the shells of the microcapsules has preferably a molecular weight ranging from 30 000 g/mol to 300 000 g/mol.

In WO 2019/175017 A1 (Givaudan) compositions comprising at least one core-shell microcapsule in a suspending medium are disclosed, wherein the shell comprises a hyperbranched polysaccharide having a specific ratio of 1,6-glycosidic bonds to 1,4-glycosidic bond selected from the group consisting of amylopectins, dextrins, hyperbranched starches, glycogen and phytoglycogen and mixtures thereof. These microcapsules show improved deposition behavior on keratinous substrates and improved rinse resistance.

US 2020/0046616 A1 (International Flavors and Fragrances) refers to polyurea and polyurethane-based capsule compositions and a method of preparing a capsule composition based on an active emulsion containing a polyisocyanate and one or more materials selected from the group consisting of a pectin, chitosan, arginine, lysine, polylysine, protein, gelatin, guar gum, and hydroxyethyl cellulose.

A microcapsule composition is disclosed in WO 2019/210125 A1 (International Flavors and Fragrances), wherein the encapsulating polymer contains a polyurea polymer that is a reaction product of a multi-functional electrophile and a multi-functional nucleophile, and wherein the multi-functional electrophile contains a polyisocyanate and the multi-functional nucleophile contains a polyamine such as chitosan having a molecular weight of >30 000 g/mol.

WO 2019/179939 A1 (Firmenich) discloses microcapsules made of an oil-based core and a shell formed from the reaction between a monomer and modified starch as the primary shell material in the presence of low levels of high molecular weight chitosan (N-acetylglucosamine polymer).

U.S. Pat. No. 5,780,060 A (Centre National de la Recherche Scientifique) relates to microcapsules comprising a wall formed of at least one plant polyphenol interfacially crosslinked with a diacid halide crosslinking agent and a protein, a polysaccharide, a polyalkylene glycol or mixture thereof co-crosslinked with the at least one plant polyphenol.

The chitosan used within the state of the art is a natural biopolymer (biomacromolecule) derived from chitin consisting of about 2 000 monomers (or monomeric units), and more specifically a polymeric aminosaccharide (also called polyaminosaccharide) consisting of β-1,4-glycosidically linked N-acetylglucosamine and D-glucosamine units. Commercially available chitosan usually exhibits a high molecular weight of at least 30 000 g/mol. As indicated above, chitosan has a limited solubility in organic acids (such as formic acid, acetic acid or citric acid) so that only a low incorporation into the shell composition of microcapsules is possible. In addition, efficient crosslinking is hindered due to the bulkiness of the substance. Thereby, the large chitosan molecules form a coating rather than a component as such in the core-shell microparticle wall construction. Consequently, for microcapsules containing such bulky polymers reduced stabilities and performances are expected.

In light of the above, the present invention was based on the complex task of providing a core-shell microcapsule as well as a process for the preparation of such microcapsules which simultaneously, on the one hand, are more environmentally friendly with increased biodegradability, and, on the other hand, which are storage-stable (especially in product formulations) and exhibit an excellent release behavior of the active ingredients, i.e., exhibit excellent performances.

It is particularly difficult to produce microcapsules that have both good stability and good release properties. The ability of the capsules to retain the active ingredient and thus to prevent the loss of volatile components depends in particular on the stability of the capsules in the product base. The shell composition and thus the stability and biodegradability of said capsule shell are primarily influenced by the starting materials used. The stability and release behavior of capsules are affected primarily by the shell composition, its morphology and thickness. Thus, capsules with good stability in particular do not automatically exhibit good biodegradability. As the degree of crosslinking increases, mostly the stability of the microcapsules increases, while at the same time the ability to biodegrade the capsule shell tends to decrease. In addition, very stable microcapsules, usually tend to exhibit a low performance as the break of the microcapsules and thus the release of the active ingredient(s) is hindered. However, if they are too unstable, they are already destroyed during storage or result in a leakage of the active ingredient and do not perform either.

Therefore, the present invention focuses on the provision of microcapsules, based on environmentally more compatible capsule wall materials which preferably show increased biodegradation properties, and which at the same time exhibit an outstanding stability as well as excellent release properties, i.e., capsule performance. It is important that not only the macromolecular material of the capsule wall itself is more environmentally compatible, but also each of the fragments produced during their disintegration.

It is therefore an object of the present invention to provide a process for the preparation of sustainable and environmentally friendly microcapsules having an increased biodegradability, in particular microcapsules as well as dispersions of such microcapsules (microcapsule slurry), which include at least one hydrophobic active ingredient, preferably a fragrance substance consisting of a single odorous compound or a mixture of odorous compounds or a perfume mixture or a cosmetic ingredient, which show a good balance of improved biodegradability, stability and performance compared to microcapsules of the state of the art.

It is also an object of the present invention to provide environmentally sustainable microcapsules with an additional benefit of increased biodegradability as well as consumer products containing said microcapsules.

These and other objects and advantages of the present invention will become clear from the following disclosure.

Surprisingly, it was found that this task can be solved by the biobased core-shell microcapsules according to the present invention, which contain crosslinked biobased or natural low molecular weight or short-chained materials that are biodegradable as such, in particular natural (amino)saccharide segments having less than 20 monomeric units, as the main building blocks in the capsule wall. The incorporation of these biobased/natural materials into the capsule shell via reaction results in more eco-friendly capsule materials with increased biodegradation properties compared to non-biobased state-of-the-art capsules. It has been found that such core-shell microcapsules containing active ingredients, such as fragrances, cosmetics, vitamins, and the like as the core, can be efficiently formed via interfacial reaction between reactive crosslinkers and functional monomeric, dimeric, oligomeric and/or short-chained polymeric (amino)saccharides and/or the corresponding thiosaccharides having one or more-OH groups and/or one or more —NH— or —NH₂ groups and/or one or more —SH groups. In addition, it was found that these microcapsules are highly stable (especially in product formulations) and show excellent release properties which are superior to those of state-of-the-art microcapsules even after long-term storage in said product formulations. These superior and balanced properties have also been surprisingly found for microcapsules having considerably reduced shell wall portions/thicknesses (see Examples 11 and 12).

### SUMMARY OF THE INVENTION

In a first aspect, the present invention relates to a biobased core-shell microcapsule, wherein the shell composition comprises or consists of a polymeric material being a reaction product of at least one polyisocyanate having at least two isocyanate groups, and preferably a mixture of at least two such polyisocyanates, and at least one saccharide and/or at least one aminosaccharide each independently having less than 20 monomeric units, preferably 15 or less monomeric units and most preferred 10 or less monomeric units (i.e., repeat units or monomeric (amino)saccharide units) and wherein the core of the capsule comprises or consists of at least one active ingredient.

In a second aspect, the present invention relates to a microcapsule slurry comprising a plurality of core-shell microcapsules according to the invention dispersed in an aqueous phase, preferably wherein the microcapsule concentration within the aqueous phase is greater than 5% and less than 70%, preferably greater than 20% and less than 60%, and most preferred greater than 30% and less than 50%.

In a third aspect, the present invention relates to a process for preparing a microcapsule slurry (i.e., a microcapsule dispersion), comprising the following steps:
(a) Providing an oil phase comprising at least one polyisocyanate having at least two isocyanate groups, and preferably a mixture of at least two such polyisocyanates, and one or more, preferably hydrophobic, active ingredient(s);
(b) Providing a first aqueous phase comprising at least one capsule formation aid;
(c) Providing a second aqueous phase comprising at least one saccharide and/or at least one aminosaccharide each independently having less than 20 monomeric units, optionally wherein the aqueous phase further comprises one or more additional capsule formation aid(s);
(d) Blending the oil phase and the first aqueous phase to obtain a preliminary oil-in-water emulsion;
(e) Blending the second aqueous phase and the preliminary oil-in-water emulsion obtained in step (d) to obtain a microcapsule slurry;
(f) Curing the microcapsules obtained in step (e).

Optionally, the process comprises an additional step (f2), wherein one or more suspension aids or structuring aids chosen from natural gums (e.g., xanthan gum, gellan gum, diutan gum, cellulose gum) or other non-gum suspension aids such as microcrystalline celluloses (Vivapur®, J. Rettenmeier & Soehne GmbH+Co) are added as thickening agents to provide for an increased suspension stability.

In another aspect, the present invention relates to a process for preparing biobased core-shell microcapsules by preparing a slurry according to the process described above, and subsequently or alternatively to step (f2) comprising the step of:
(g) Isolating the core-shell microcapsules from the slurry and optionally, drying the as-obtained isolated core-shell microcapsules.

In yet another aspect, the present invention relates to a slurry comprising core-shell microcapsules (i.e., a microcapsule dispersion) obtained by the process according to the invention as described above. Moreover, the present invention also relates to core-shell microcapsules obtained by the process according to the invention as such.

In addition, the present invention relates to the use of the inventive core-shell microcapsules and/or the inventive slurry comprising the core-shell microcapsules for the preparation of a variety of consumer products.

Finally, the present invention relates to consumer products comprising the biobased core-shell microcapsules according to the invention or the microcapsule slurry according to the invention, wherein the consumer product is selected amongst others from the group consisting of: cosmetics, personal care products, in particular skin cleaning and skin care products, shampoos, rinse-off conditioners, deodorants, antiperspirants, body lotions, textile care product and, homecare/household products, in particular liquid detergents, all-purpose cleaners, laundry and cleaning agents, fabric softeners, scent boosters, fragrance enhancers in liquid or solid form as well as pharmaceuticals.

Surprisingly, it was found that the biobased core-shell microcapsules according to the invention show increased biodegradability and at the same time exhibit high mechanical, thermal and chemical stabilities as well as excellent release properties.

These and other aspects, features and advantages of the present invention become apparent to the person skilled in the art by studying the following detailed description, figures, and the patent claims. Each feature from one aspect of the invention can be used or exchanged in another aspect of the invention. The examples contained herein illustrate the invention without restricting it.

The term “saccharide” as used herein is to be understood to encompass the features “saccharide”, “aminosaccharide” and/or “thiosaccharide”, meaning, for example, when referring to “saccharide-based microcapsules” all variants of microcapsules being formed using the building blocks as defined herein are meant, i.e., capsules formed based on the polymerization of saccharide building blocks, aminosaccharide building blocks and/or thiosaccharide building blocks each independently having less than 20 monomeric units, preferable 15 or less monomeric units and even more preferred 10 or less monomeric units.

The terms “at least one” or “one or more” as used herein refer to 1 or more and are used synonymously. The terms “at least two” and “two or more” or “more than one” as used herein refer to two, three, four or more, and are used synonymously.

Numerical examples given in the form “from x to y” include the values mentioned. If several preferred numerical ranges are given in this format, all ranges resulting from the combination of the different endpoints are also included.

### FIGURES

FIG. 1A: FIG. 1A is a diagram showing the results of a sensory evaluation of the release properties of state-of-the-art microcapsules (according to Comparative Example 13; polyurea microcapsules) and microcapsules according to Example 1 in comparison to a pure fragrance oil reference in a fabric softener formulation, each aged for one week in the softener, after machine drying.

FIG. 1B: FIG. 1B is a diagram showing the results of a sensory evaluation of the release properties of state-of-the-art microcapsules (according to Comparative Example 13; polyurea microcapsules) and microcapsules according to Example 1 in comparison to a pure fragrance oil reference in a fabric softener formulation, each aged for one week in the softener, after line drying.

FIGS. 2A and 2B: FIGS. 2A and 2B are diagrams showing the results of a sensory evaluation of the release properties of microcapsules according to Example 2 in comparison to a pure fragrance oil reference in a fabric softener formulation, each aged for one week in the softener, after machine drying and after line drying, respectively.

FIGS. 3A and 3B: FIGS. 3A and 3B are diagrams showing the results of a sensory evaluation of the release properties of microcapsules according to Example 3 in comparison to a pure fragrance oil reference in a fabric softener formulation, each aged for one week in the softener, after machine drying and after line drying, respectively.

FIGS. 4A and 4B: FIGS. 4A and 4B are diagrams showing the results of a sensory evaluation of the release properties of microcapsules according to Example 4 in comparison to a pure fragrance oil reference in a fabric softener formulation, each aged for one week in the softener, after machine drying and after line drying, respectively.

FIGS. 5A and 5B: FIGS. 5A and 5B are diagrams showing the results of a sensory evaluation of the release properties of microcapsules according to Example 5 in comparison to a pure fragrance oil reference in a fabric softener formulation, each aged for one week in the softener, after machine drying and after line drying, respectively.

FIG. 6A: FIG. 6A is a diagram showing the results of a sensory evaluation of the release properties of state-of-the-art microcapsules (according to Comparative Example 13; polyurea microcapsules) and microcapsules according to Example 6 in comparison to a pure fragrance oil reference in a fabric softener formulation, each aged for one week in the softener, after machine drying.

FIG. 6B: FIG. 6B is a diagram showing the results of a sensory evaluation of the release properties of state-of-the-art microcapsules (according to Comparative Example 13; polyurea microcapsules) and microcapsules according to Example 6 in comparison to a pure fragrance oil reference in a fabric softener formulation, each aged for one week in the softener, after line drying.

FIG. 6C: FIG. 6C is a diagram showing the results of a sensory evaluation of the microcapsules according to Example 6 in comparison to a pure fragrance oil reference in a fabric softener formulation, each aged for four weeks in the softener, after machine drying.

FIG. 6D: FIG. 6D is a diagram showing the results of a sensory evaluation of the microcapsules according to Example 6 in comparison to a pure fragrance oil reference in a fabric softener formulation, each aged for four weeks in the softener, after line drying.

FIGS. 7A and 7B: FIGS. 7A and 7B are diagrams showing the results of a sensory evaluation of the release properties of microcapsules according to Example 7 in comparison to a pure fragrance oil reference in a fabric softener formulation, each aged one week in the softener, after machine drying and after line drying, respectively.

FIGS. 8A to 8D: FIGS. 8A and 8B are diagrams showing the results of a sensory evaluation of the release properties of microcapsules according to Example 8 in comparison to a pure fragrance oil reference in a fabric softener formulation, each aged for one week in the softener, after machine drying and after line drying, respectively, while FIGS. 8C and 8D show the corresponding results of a sensory evaluation of the release properties of capsules aged for four weeks in the softener.

FIGS. 9A and 9B: FIGS. 9A and 9B are diagrams showing the results of a sensory evaluation of the release properties of two microcapsule samples according to Example 9 (Examples 9A and 9B) in comparison to a pure fragrance oil reference in a fabric softener formulation, each aged for one week in the softener, after machine drying and after line drying, respectively.

FIGS. 9C and 9D: FIGS. 9C and 9D are diagrams showing the results of a sensory evaluation of the release properties of two microcapsule samples according to Example 9 (Examples 9A and 9B) in comparison to a pure fragrance oil reference in a fabric softener formulation, each aged for four weeks in the softener, after machine drying and after line drying, respectively.

FIGS. 10A to 10D: FIGS. 10A and 10B are diagrams showing the results of a sensory evaluation of the release properties of microcapsules according to Example 10 in comparison to a pure fragrance oil reference in a fabric softener formulation, each aged for one week in the softener, after machine drying and after line drying, respectively, while FIGS. 10C and 10D show the corresponding results of a sensory evaluation of the release properties of capsules aged for four weeks in the softener after drying.

FIG. 11A: FIG. 11A is a diagram showing the results of a sensory evaluation of the release properties of state-of-the-art microcapsules (according to Comparative Example 13; polyurea microcapsules) in comparison to microcapsules according to Examples 11 and 12 in a fabric softener formulation, each aged for one week in the softener, after line drying.

FIG. 11B: FIG. 11B is a diagram showing the results of a sensory evaluation of the release properties of state-of-the-art microcapsules (according to Comparative Example 13; polyurea microcapsules) in comparison to microcapsules according to Examples 11 and 12 in a fabric softener formulation, each aged for four weeks in the softener, after line drying.

FIGS. 12A to 12D: FIGS. 12A and 12B are diagrams showing the results of a sensory evaluation of the release properties of microcapsules according to Example 12 in comparison to a pure fragrance oil reference in a fabric softener formulation, each aged for one week in the softener, after machine drying and after line drying, respectively, while FIGS. 12C and 12D show the corresponding results of a sensory evaluation of the release properties of capsules aged for four weeks in the softener after drying.

FIG. 13: FIG. 13 is a diagram showing the increased biodegradability of microcapsules according to Example 1 (Sample #1) according to OECD 301F in comparison with sodium benzoate and a toxicity control (mixture of the microcapsules according to the invention and sodium benzoate) during the incubation period which was prolonged to 45 days.

FIG. 14: FIG. 14 is a diagram showing the increased biodegradability of microcapsules according to Example 1 (Sample #2) according to OECD 301F in comparison with sodium benzoate and a toxicity control (mixture of the microcapsules according to the invention and sodium benzoate) during the incubation period of 28 days.

FIG. 15: FIG. 15 is a diagram showing the biodegradability results of fully synthetic polyurea-based state-of-the-art microcapsules according to OECD 301F prepared according to Comparative Example 13 and corresponding to the capsules disclosed in U.S. Pat. No. 6,586,107 B2.

FIGS. 16A to 16M: FIGS. 16A to 16M are diagrams showing the distribution of the median particle size by volume (Dv(50)) of microcapsules according to Examples 1 to 12 and microcapsules according to Comparative Example 13 (polyurea microcapsules) within the corresponding microcapsule slurries. FIG. 16I corresponds to Example 9A.

FIG. 17: FIG. 17 is a diagram showing the increased biodegradability of microcapsules according to Example 6 according to OECD 301F in comparison with sodium benzoate and a toxicity control (mixture of the microcapsules according to the invention and sodium benzoate) during the incubation period.

FIG. 18: FIG. 18 is a diagram showing the increased biodegradability of microcapsules according to Example 7 according to OECD 301F in comparison with sodium benzoate and a toxicity control (mixture of the microcapsules according to the invention and sodium benzoate) during the incubation period.

FIG. 19: FIG. 19 is a diagram showing the increased biodegradability of microcapsules according to Example 8 according to OECD 301F in comparison with sodium benzoate and a toxicity control (mixture of the microcapsules according to the invention and sodium benzoate) during the incubation period.

### DETAILED DESCRIPTION OF THE INVENTION

In a first aspect, the present invention relates to biobased or (amino)saccharide-based core-shell microcapsules, wherein the shell comprises or consists of a polymeric material being a reaction product of at least one polyisocyanate having at least two isocyanate groups, and preferably a mixture of at least two polyisocyanates each having at least two isocyanate groups, and at least one saccharide and/or at least one aminosaccharide each independently having less than 20 monomeric units, i.e., comprising or preferably consisting of less than 20 monomeric units (monosaccharide units and/or monomeric aminosaccharide units), and wherein the core comprises or consists of at least one active ingredient, such as a fragrance compound, which is preferably hydrophobic in nature.

Preferably, the saccharide and/or aminosaccharide building block or component is a mono-, di- and/or (straight or branched) oligosaccharide and/or a monomeric, dimeric and/or (straight or branched) oligomeric aminosaccharide (also known as oligoaminosaccharide) or a mixture of these monomeric, dimeric or oligomeric compounds in any combination. However, also short-chained polymeric structures are suitable as building blocks as long as they comprise or consist of less than 20 monomeric units and preferably 15 or less monomeric units and even more preferred 10 or less monomeric units.

Thus, additionally, short-chained (straight or branched) polysaccharides (polymeric saccharides) and/or polyaminosaccharides (polymeric aminosaccharides) with less than 20 monomeric units, i.e., having equal to 19 or less monomeric units, are suitable and can be used as building blocks alone in any combination or in combination with the aforementioned monomeric, dimeric or oligomeric compounds for the formation of a capsule wall according to the invention.

In the context of the present invention, microcapsules are microparticles produced by interfacial polymerization which contain at least one or more active ingredient(s) as core material in the interior of the capsule and which are enclosed by a capsule shell or capsule wall. The active ingredients are preferably hydrophobic or lipophilic active ingredients. Such active ingredients are not soluble or are only poorly soluble in water, but are highly soluble in fats and oils. The terms “microcapsule” and “capsule” are used synonymously within the context of the present invention as well as the terms “hydrophobic” and “lipophilic” and the terms “shell” and “wall”.

In the context of the present invention, the capsule shell or capsule wall is preferably based on saccharides and/or aminosaccharides, as specified herein, reacted and crosslinked by isocyanates to produce a stable and highly efficient but simultaneously increasingly biodegradable capsule shell with excellent release properties, i.e., release performance.

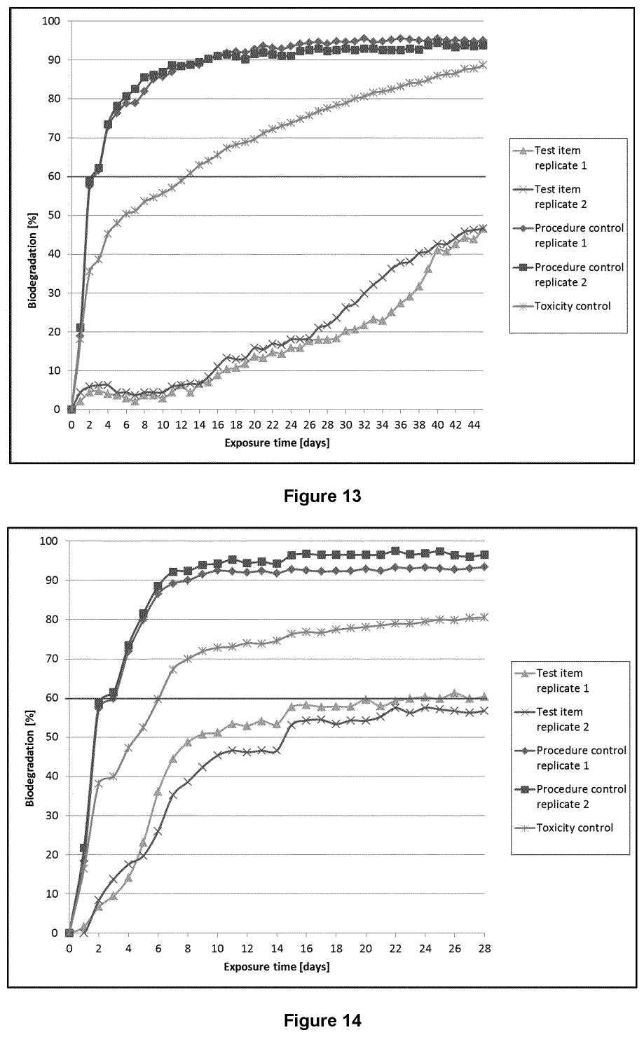

It was surprisingly found that the inventive biobased/(amino)saccharide-based core-shell microcapsules possess increased biodegradation properties according to the standardized OECD 301F test procedure compared to the-state-of-art microcapsules based on fully synthetical structures and more specifically guanidine-based building blocks (see FIGS. 13 to 15 and 16 to 19). While the fully synthetic capsules according to the state of the art show nearly no biodegradability at all, the inventive capsule shells show a considerably increased biodegradability according to the OECD 301F biodegradability standard.

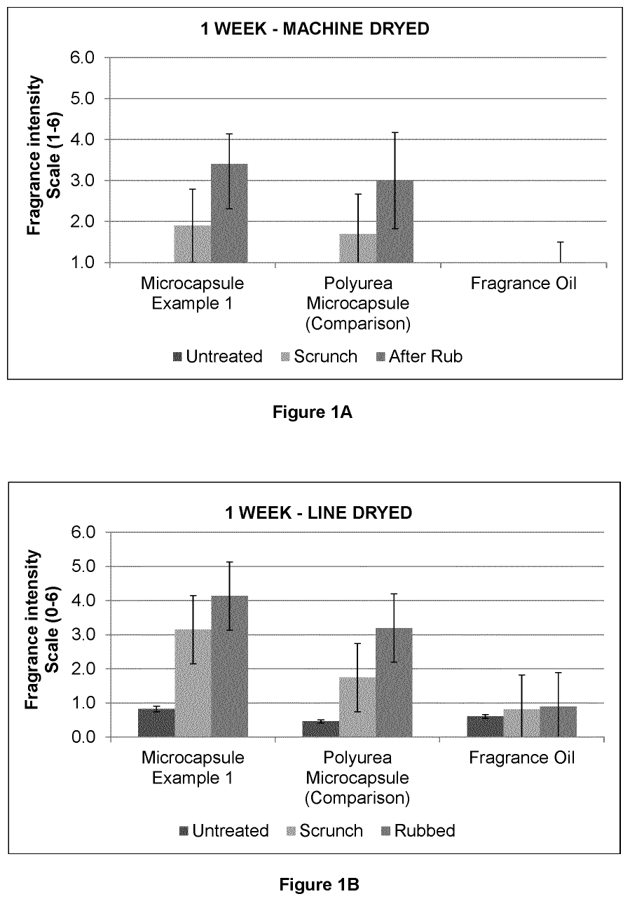

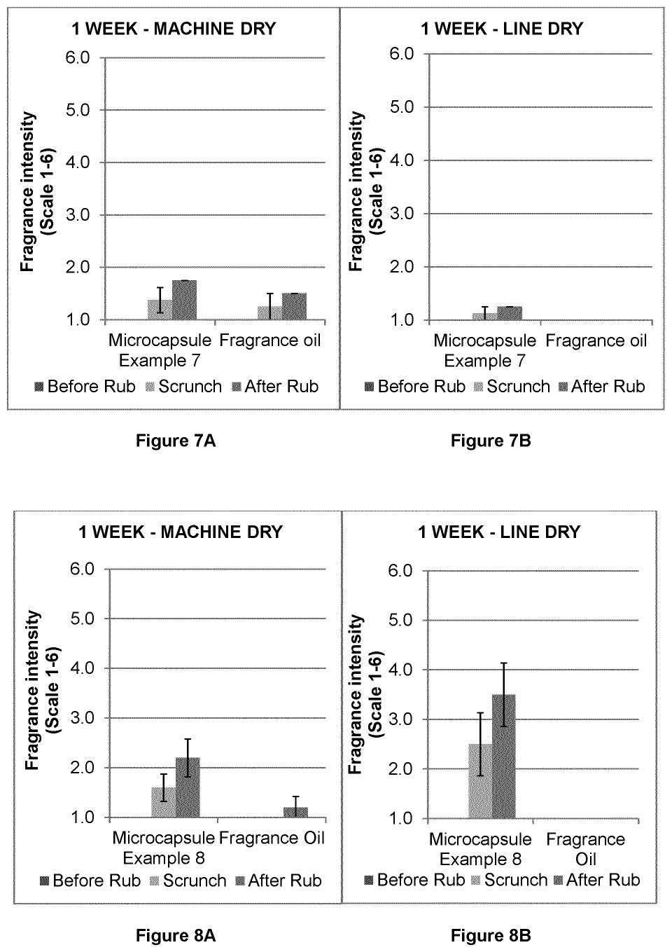

Simultaneously, the inventive microcapsules exhibit a high mechanical and thermal stability against mechanical influences, such as those prevailing in a tumble dryer or heat, as well as a high chemical stability for example within product formulations, but at the same time allow for an efficient targeted, signal-induced release of the active ingredient(s). In addition, sensory experiments have shown that, in the case of fragrances as active ingredients, high fragrance intensities could be perceived, which suggests that the fragrances were efficiently enclosed within the inventive core-shell microcapsules indicating the efficient reduction of losses due to diffusion of the active ingredient out of the microcapsules (see FIGS. 1 to 12). Simultaneously, this also demonstrates the efficient balance between performance and stability of said capsules. Moreover, the inventive microcapsules showed an increased stability within consumer product formulations such as fabric softeners of at least four weeks. Consequently, it was possible to produce highly stable microcapsules which allow for an efficient encapsulation of active ingredients and simultaneously high performances despite being based on biobased components contrary to the teaching of the state of the art.

Thereby, it was found that the (amino)saccharide building blocks (and/or thiosaccharide building blocks) serve as sites for biological attack and degradation, thus significantly reducing the overall environmental impact of the microcapsules according to the invention. Consequently, the inventive biobased core-shell microcapsules differ from state-of-the-art microcapsules primarily in their considerably higher biodegradability and thus are more suitable for the development of more environmentally friendly but efficient consumer product formulations.

Consequently, in a preferred variant, the present invention relates to core-shell microcapsules according to the first aspect, wherein the microcapsules show increased biodegradability compared to state-of-the-art microcapsules and are preferably inherently biodegradable, and more preferably readily biodegradable, thus showing increased biodegradation properties according to the OECD 301F biodegradability standard (manometric respirometry test). For some samples the test was prolonged to 45 days in accordance with the official test procedure.

However, preferably a mixture of two or more different polyisocyanates is used for the formation of the capsule wall, i.e., as the isocyanate component or crosslinker. These result in more stable and better performing microcapsules and thus in improved capsule properties. Moreover, it has been observed that, for example, polyisocyanates having an aliphatic structure result in more stable core-shell microcapsules allowing for the efficient encapsulation of, for example, volatile active ingredients such as fragrance materials and that the combination of two or more different polyisocyanates is advantageous in terms of capsule properties, especially when at least one of these polyisocyanates is an aliphatic polyisocyanate or alternatively, when all of the polyisocyanates comprised in the mixture are aliphatic in nature (see for example Examples 3 and 4), allowing for the formation of capsules with improved properties such as stability and performance, i.e., release behavior.

Thus, in an alternative embodiment, core-shell microcapsules are disclosed, wherein at least one of the one or more polyisocyanates having at least two isocyanate groups, i.e., two or more isocyanate functionalities, comprises an aliphatic structure, as will be specified herein, and preferably a cycloaliphatic structure. Therefore, preferably at least one aliphatic polyisocyanate having at least two isocyanate groups is used for the preparation of the core-shell microcapsules according to the invention. Thereby, this aliphatic polyisocyanate can be either used in mixture with other aliphatic polyisocyanates or alternatively in mixture with aromatic polyisocyanates or even in mixture with further aliphatic and aromatic polyisocyanates.

Therefore, one embodiment of the present invention relates to microcapsules according to the invention being prepared using one or more aliphatic polyisocyanates or comprising the reaction product of one or more aliphatic polyisocyanates with at least one saccharide and/or at least one aminosaccharide having less than 20 monomeric units, respectively.

In another preferred embodiment the capsules are prepared using a mixture of at least one aliphatic polyisocyanate and at least one aromatic polyisocyanate resulting in highly efficient microcapsules.

Thereby, preferably, the molar or weight ratio, and even more preferred the molar ratio of the at least one aliphatic polyisocyanate to the at least one aromatic polyisocyanate is ranging from 85:15, and even more preferred from 90:10, to 99:1 for microcapsules preferably being based on or comprising aminosaccharides preferably having more than one monomeric unit (see Example 11). Thus, the molar or weight ratio, and even more preferred the molar ratio of the at least one aliphatic polyisocyanate to the at least one aromatic polyisocyanate is preferably higher than 85:15 and preferably higher than 90:10 for microcapsules preferably being based on or comprising aminosaccharides preferably having more than one monomeric unit. Based on these ratios highly stable but at the same time excellently performing capsules (in terms of the targeted release behavior) can be prepared, showing an increased stability within product formulation.

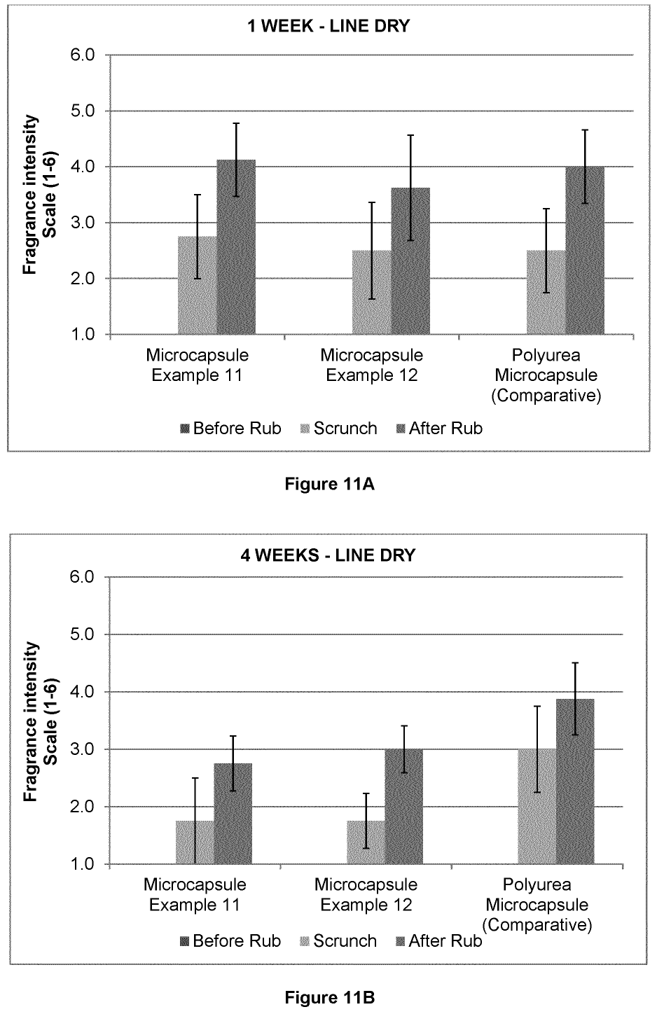

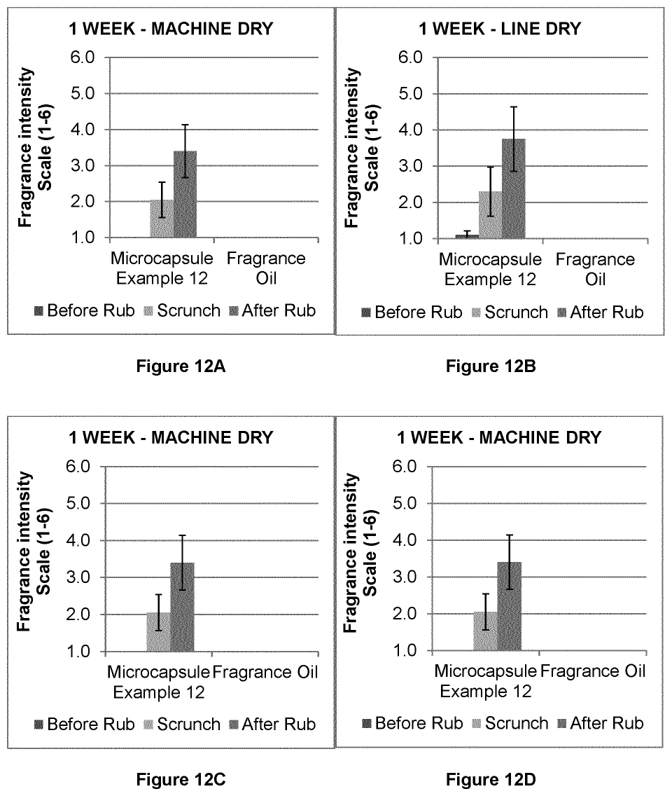

Preferably, if mixtures of two or more polyisocyanates are used, it is preferred that said mixture comprises or consist of more than 80 mol-% of aliphatic components/polyisocyanates, i.e., the molar fraction of the aliphatic components/polyisocyanates is preferably more than 80% and even more preferred at least 81%, and further preferred at least 85 mol-% and most preferred at least 90 mol-% in the mixture of isocyanates for the microcapsules being based on or comprising aminosaccharides having more than one monomeric unit. It was surprisingly found that microcapsules having such high molar contents of aliphatic isocyanates relative to the total isocyanate content show excellent stabilities and performances even after four-week agent in liquid product formulations such as fabric softeners and thus an improved balance of the overall capsule properties. These excellent properties are particularly surprising for microcapsules having considerably reduced shell contents, i.e., considerably reduced shell thicknesses (see Examples 11 and 12 and FIGS. 11 to 12).

Enhanced crosslinking and thus polymerization with the polyisocyanate(s) could be achieved for saccharides and/or aminosaccharides each independently having preferably 15 or less, and even more preferred 10 or less monomeric units resulting in more stable and better performing capsules. In addition, for these capsules an enhanced balance between stability, release performance and biodegradability could be observed. The same is true for the corresponding thiosaccharides. Even more preferred the one or more saccharide and/or one or more aminosaccharide is monomeric and most preferred it is glucosamine.

Therefore, core-shell microcapsules are particularly preferred, wherein the at least one saccharide and/or aminosaccharide has 10 or less monomeric units.

In order to achieve an efficient encapsulation based on the polymerization of the building blocks, i.e., the isocyanate component(s) and the (amino)saccharide component(s), it is further necessary, that the at least one saccharide and/or at least one aminosaccharide contains at least two functional groups independently selected from the group consisting of: primary and secondary hydroxyl groups (—OH) as well as primary and secondary amine groups (—NH₂, —NH—) allowing for the formation of polyurethane and/or polyurea-based linkages.

Alternatively, or in combination with the above (amino)saccharides thiosaccharides can be suitably used, which have one or more functional thiol groups (—SH) allowing for the formation of polythiourethane structures/linkages or mixtures of these with the aforementioned polyurethane and/or polyurea-based linkages between the building blocks.

The inventive eco-friendly core-shell microcapsules allow for the efficient encapsulation of a variety of active ingredients and thus allow for the incorporation into a wide range of consumer product formulations.

Suitable active ingredients are for example cosmetic active ingredients, such as hair conditioning agents or active skin-product ingredients such as skin moisturizing agents, wrinkle control agents, skin conditioning agents, skin lightening agents, anti-acne agents and the like, as well as fragrances or perfumes, meaning single fragrance or perfume substances (also called odorous substances or fragrant substances), i.e., chemical compounds having a smell or odor and thus all natural and synthetic substances that impart an olfactorily perceptible odor, or compositions of one or more fragrance or perfume substances, i.e., mixtures of the aforementioned compounds or mixtures comprising said compounds, and/or other perfume components, perfume oils, aroma substances, and/or aromas. Further suitable active ingredients are, for example, agrochemicals, active pharmaceutical ingredients, dyes, UV-active substances, optical brighteners, bodying agents, drape and form control agents, smoothness agents, static control agents, wrinkle control agents, sanitizing agents, disinfecting agents, germ control agents, mold control agents, mildew control agents, antiviral agents, antimicrobials, drying agents, stain resistance agents, soil release agents, malodor control agents, fabric freshening agents, dye fixatives, color maintenance agents, color restoring/rejuvenating agents, anti-fading agents, anti-abrasion agents, wear resistance agents, fabric integrity agents, anti-wear agents, rinsing aids, UV-protection agents, sun fade inhibitors, insect repellents, anti-allergenic agents, flame retardants, water-proofing agents, fabric softening agents, shrinkage resistance agents and/or stretch resistance agents, fluorescent paints, solvents, waxes, silicone oils, lubricants, cooling agents, TRPV-modulators as well as mixtures of the above-mentioned active ingredients.

However, preferably the active ingredient is a fragrance substance/perfume substance or composition as defined above, i.e., a substance or mixture of substances that impart an olfactorily perceptible odor, a perfume oil or cosmetic ingredient and preferably these fragrance/perfume substances or compositions encapsulated by the inventive core-shell microcapsules are imparting a pleasant odor to a consumer product.

The core-shell microcapsules according to the invention preferably have a median particle size by volume (Dv(50)) of 1 μm to 100 μm, preferably of 5 μm to 55 μm and most preferred 5 μm to 50 μm, within the microcapsule dispersion, i.e. the microcapsule slurry. Thereby, the Dv(50) value refers to the volume distribution of particles and is defined as the particle diameter at which half of the population lies below this value.

The diameter of the microcapsule is preferably determined by laser diffraction using a Malvern Mastersizer 3000 (according to the manufactures instructions). Thereby the measured intensity values of the scattered light are calculated into a Dv(50) value using a mathematical model, such as and preferably by Fraunhofer diffraction/approximation. The Dv(50) value is the particle size at which 50 vol.-% of the microcapsules are finer than the Dv(50) value and 50 vol.-% are coarser. Accordingly, the Dv(10) and Dv(90) values indicate that 10% and 90%, respectively, of the total volume of capsule in the sample are smaller than these values.

If the median particle size of the core-shell microcapsules is within this range, the deliverance of actives in applications such as fabric, skin and personal care products is enhanced. Larger particles, in particular particles larger than 100 μm, are less effective regarding the deliverance of actives to surfaces and cannot be stably incorporated into the product formulation and might aggregate. Moreover, they are more difficult to suspend in aqueous formulations or product formulations and result in a more tactile feel (roughness), which is disadvantageous for most of the intended applications. In addition, if the particle size is within the range defined above, enhanced balances between stability and release performances could be achieved.

Preferably however, the inventive microcapsules are obtained and stored in form of a slurry, i.e., as finely dispersed microcapsules in an aqueous phase (microcapsule dispersion). The usage of such a slurry facilitates the dosage and incorporation of the microparticles into product formulations and allows for an enhanced storage without accumulation of the particles or oxidation and suppresses the evaporation of volatile components. Moreover, the usage of such dispersions allows for an efficient release of the active ingredients by external forces such as friction, heat or an environmental change. In addition, the stability of the actives against the environmental conditions of the consumer product formulations (pH of the consumer product base etc.) can be enhanced up to the release of the actives based on the receipt of a release signal (friction, change in pH, heat, etc.).

The microcapsule slurry comprising a plurality of the inventive biobased core-shell microcapsules can be effectively incorporated in a variety of different applications, in particular into liquid (product) formulations. The inventive core-shell microcapsules exhibit a high stability within the dispersion (slurry) and allow for long storage times and easier incorporation and dosage into consumer product formulations.

Therefore, another object of the present invention relates to a microcapsule slurry comprising a plurality of core-shell microcapsules according to the invention dispersed in an aqueous phase, preferably wherein the microcapsule concentration within the aqueous phase is greater than 5% and less than 70% by weight, preferably greater than 20% and less than 60% by weight, most preferably greater than 30% and less than 50% by weight.

In a further aspect the present invention relates to a process for the preparation of a microcapsule slurry according to the invention. The core-shell microcapsule slurry, and subsequently also the isolated core-shell microcapsules, can be obtained based on the following steps:
(a) Providing an oil phase comprising at least one polyisocyanate having at least two isocyanate groups, i.e., two or more isocyanate functionalities, such as one or more linear or branched aliphatic and/or aromatic polyisocyanate(s) or a mixture thereof, and one or more preferably hydrophobic active ingredient(s);
(b) Providing a first aqueous phase comprising at least one capsule formation aid;
(c) Providing a second aqueous phase comprising at least one saccharide and/or at least one aminosaccharide each independently having less than 20 monomeric units, preferably 15 or less monomeric units, even more preferred 10 or less monomeric units, preferably having at least two functional reactive groups independently selected from the group consisting of: primary and secondary hydroxyl groups (—OH) as well as primary and secondary amine groups (—NH₂, —NH—), optionally wherein the second aqueous phase further comprises one or more additional formation aid(s);
(d) Blending the oil phase and the first aqueous phase to obtain a preliminary oil-in-water emulsion;
(e) Blending the second aqueous phase and the preliminary oil-in-water emulsion obtained in step (d) to obtain a microcapsule slurry; and
(f) Curing the microcapsules obtained in step (e) within the microcapsule slurry.

Optionally, the process comprises an additional step (f2), wherein one or more suspension aids or structuring aids chosen from natural gums (e.g., xanthan gum, gellan gum, diutan gum, cellulose gum and preferably diutan gum) are added as thickening agents to provide for an increased suspension stability. Such substances spontaneously form colloidal dispersions with water and thus stabilize suspensions. Suitable suspension aids are, for example, Kelco-Vis™ DG and Keltrol® from CP Kelco, carboxymethyl cellulose (CMC) and Vivapur™ (microcrystalline cellulose) from J. Rettenmaier & Soehne GmbH+Co KG.

In the following, the single process steps as well as suitable components according to the first aspect are disclosed in more detail.

In a first stage of the process according to the invention, a preliminary oil-in-water emulsion is formed. For this purpose, an internal non-aqueous phase, and more specifically an oil phase is provided which comprises or consists of at least one polyisocyanate with two or more isocyanate groups and at least one preferably hydrophobic active ingredient which is to be encapsulated.

The at least one isocyanate used in the production process described herein or in the core-shell microcapsules according to the first aspect has at least two isocyanate groups for forming polymeric networks, i.e., the capsule shell or capsule wall by polymerization. Thereby, the di- and/or higher polyisocyanates, respectively, form stable polymeric structures based on polyurethane and/or polyurea-based chemical linkages by interfacial polymerization with the functional groups of the at least one saccharide and/or aminosaccharide each independently having less than 20 monomeric units as specified herein, efficiently encapsulating the hydrophobic core comprising the active ingredient by forming an efficient but simultaneously biobased capsule wall showing a higher biodegradability compared to fully synthetic capsules according to the state of the art.

In the case of thiosaccharides accordingly thiourethane-based linkages are formed in addition to or alternative to the above linkages. Suitable thiosaccharides are for example N-acetylcysteine, 5-thio-D-glucose or methyl 6-thio-6-deoxy-α-D-galactopyranoside. Further suitable substances are however cysteine, homocysteine and glutathione.

In a preferred variant, the at least one polyisocyanate having at least two isocyanate groups, i.e., two, three or more functional isocyanate groups, is preferably selected from the group consisting of: linear, cyclic, and branched aliphatic and aromatic polyisocyanates as well as mixtures thereof. Thereby, the di- and/or higher polyisocyanates can comprise monomeric, dimeric, oligomeric or polymeric structures having different chain lengths. For example, di- and polyisocyanates often self-react to form dimers (uretidiones), trimers (isocyanurates) or higher isocyanate oligomers. In addition, the polyisocyanates can form adducts, such as Takenate™ D-110N and D-120N by Mitsui Chemicals which are aliphatic polyisocyanate adduct prepolymers.

Within the context of the present invention, the term “isocyanate” as used herein refers to the functional group having the formula R—N═C═O. A compound that has at least two isocyanate groups, i.e., two or more isocyanate groups, is referred to as a polyisocyanate.

A molecule described herein by the term “aliphatic polyisocyanate” refers to any polyisocyanate that is non-aromatic and might be linear, branched or cyclic, i.e., cycloaliphatic. In these compounds the functional isocyanate groups are not directly attached to an aromatic ring. However, the aliphatic isocyanate(s) can comprise aromatic structures. Moreover, the polyisocyanate can be of aromatic nature (“aromatic polyisocyanates”), i.e., structures wherein the functional isocyanate groups are directly attached to an aromatic ring. The polyisocyanates used within the scope of the present invention further may exhibit any substitutions, including for instance aliphatic substituents, aromatic substituents, heteroatoms, such as a halogen, particularly fluorine, chlorine, bromine and/or iodine, and/or other functional groups, such as alkoxy groups. Thereby, the cyclic structures can also be heterocyclic or heteroaromatic.

In a preferred variant, the at least one polyisocyanate is therefore, preferably selected from the group consisting of: monomeric, dimeric or oligomeric (i.e. interlinked polyisocyanates) linear or branched aliphatic polyisocyanates, cycloaliphatic (also heterocyclic) polyisocyanates, aromatic (or heteroaromatic) polyisocyanates, their substitution products as well as mixtures of the above-mentioned monomeric, dimeric or oligomeric compounds, however, aliphatic compounds or mixtures of aliphatic and/or aromatic polyisocyanates being preferably used.

Preferably, a mixture of at least two different polyisocyanates is used for the preparation of the inventive microcapsules resulting in microcapsules with improved balance of the capsule properties. Even more preferred, a mixture of two different polyisocyanates is used.

Alternatively, or additionally, the corresponding polyisothiocyanates can be used for the formation of the capsule wall as well as compounds comprising a mixture of both, i.e., at least one isocyanate and at least one isothiocyanate functionality.

According to a preferred embodiment of the present invention, the linear or branched aliphatic polyisocyanate(s) is or are selected from the group consisting of pentamethylene diisocyanate (PDI, such as Stabio D-370N or D-376N by Mitsui Chemicals Inc., Japan), hexamethylene diisocyanate (HDI) such as Desmodur N-3400 (HDI-uretdione; Covestro Corp.), Bayhydur® (Covestro Corp.), ethyl ester lysine triisocyanate, lysine diisocyanate ethyl ester and derivatives thereof, preferably wherein each of the derivatives comprises more than one isocyanate group and optionally further comprises one or more groups selected from the group consisting of biuret, isocyanurate, uretdione, iminooxadiazinedione and trimethylol propane adduct, and/or wherein the cyclic aliphatic polyisocyanate(s) is or are selected from the group consisting of isophorone diisocyanate (IPDI), 1,3-bis(isocyanatomethyl)cyclohexane (H6XDI, such as Takenate 600 by Mitsui Chemicals Inc., Japan), 1,2-bis(isocyanatomethyl)cyclohexane, 1,4-bis(isocyanato-methyl)cyclohexane, methylenebis(cyclohexyl isocyanate) (H12MDI) and derivatives thereof, preferably wherein each of the derivatives comprises more than one isocyanate group and optionally further comprises one or more groups selected from the group consisting of biuret, isocyanurate, uretdione, iminooxadiazinedione and trimethylol propane adduct (such as TMP adduct of H6XDI, particularly Takenate D-120N by Mitsui Chemicals Inc., Japan).

Also preferred is/are aliphatic polyisocyanate(s) obtained from renewable resources, such as PDI (Stabio D-370N or D-376N by Mitsui Chemicals Inc., Japan). It has been found that such aliphatic polyisocyanates obtained from renewable resources do not impede the quality/properties of the core-shell capsules. It was found that, for example, Stabio D-370N polyisocyanates contain approximately 70% biobased carbon, i.e., renewable, and non-fossil organic carbon and approximately only 30% fossil-based carbon relative to the total carbon of the substance indicated by the ¹⁴C (radiocarbon) content.

Biobased or bio-derived reagents are preferred in view of health and environmental aspects. Biobased materials (bio-derived materials) refer to compounds containing organic carbon of renewable origin such as from agricultural, plant, animal, fungi, microorganism, marine or forestry materials living in a natural environment in equilibrium with the atmosphere, being of non-fossil origin such as petroleum as defined according to the ASTM D6866 standard test. Further it is defined that such biogenic compounds are compounds containing carbon (organic and inorganic) of renewable origin like agricultural, plant, animal, fungi, microorganisms, microorganisms, marine, or forestry materials. Preferably, the inventive core-shell microcapsules comprise such biogenic or biobased, i.e., bio-derived materials. Therefore, bio-derived materials are preferably used for the preparation of core-shell microcapsules according to the invention.

Thus, in a further preferred variant of the present invention preferably bio-based polyisocyanates such as STABiO™ and/or bio-derived (amino)saccharides are used, i.e., building blocks which are considered natural and renewable, and which are from a sustainable feedstock i.e., a bio-derived feedstock that is not mainly petrochemically-derived.

Polymerizable aliphatic isocyanates are particularly preferable in this context due to their chemical affinity to biobased systems. For example, both lysine and 1,5-diisocyanatopentane show the same degradation product, 1,5-diaminopentane, and are therefore particularly suitable for use in the production of biobased and biodegradable microcapsules, taking environmental considerations into account.

Therefore, in a preferred embodiment of the present invention preferably at least one aliphatic polyisocyanate is used.

Preferably, derivatives of the linear, branched and/or cyclic aliphatic polyisocyanates are employed. A derivative as used herein is understood in its broadest meaning as a compound that is derived from a compound by a chemical reaction. Examples of derivatives encompass oligomers and/or adducts of the above mentioned linear or branched aliphatic or cycloaliphatic polyisocyanate(s). Preferred oligomers are biurets, isocyanurates, uretdiones, iminooxadiazinediones and preferred adducts are trimethylol propane adducts. These oligomers/adducts are well known in the art and disclosed for instance in U.S. Pat. No. 4,855,490 A or U.S. Pat. No. 4,144,268 A. Preferably, the aliphatic polyisocyanate is present in a monomeric form and/or dimerised form or in an oligomeric form.

Said derivatives of the linear, branched or cyclic aliphatic polyisocyanates may also be obtained by reaction of said polyisocyanates with polyalcohols (e.g., glycerine), polyamines, polythiols (e.g., dimercaprol), and/or mixtures thereof.

Within the framework of this text, aromatic polyisocyanates are compounds, wherein two or more isocyanate residues are directly bound to aromatic C-atoms, and derivatives thereof, wherein each of the derivatives comprises more than one isocyanate group and further comprises one or more groups selected from the group consisting of biuret, isocyanurate, uretdione, iminooxadiazinedione and trimethylol propane adducts and the like.

Examples of aromatic di- or polyisocyanates include compounds such as monomeric diphenylmethane-2,4′- or -4,4′-diisocyanate (MDI) and oligomeric/polymeric forms thereof (PMDI) or mixtures thereof, 2,4- and/or 2,6-toluene diisocyanate (TDI) and 1,5- or 1,8-naphthalene diisocyanate (NDI).

Among the polyisocyanates, diisocyanates are particularly preferred and are therefore primarily used in the context of the present invention, i.e., isocyanate compounds having two functional isocyanate groups. Therefore, suitable polyisocyanates are, for example, methylenediphenyl diisocyanate (MDI; all isomers and derivatives); toluol diisocyanate (TDI); hexamethylene diisocyanate (HDI; all isomers and derivatives); isophorone diisocyanate (IPDI); 4,4-dicyclohexylmethane diisocyanate (H12MDI); 1,5-pentamethylene diisocyanate (PDI; Stabio™); 1,3-bis(isocyanatomethyl)cyclohexane (Takenate™ 600); hydrophilic modified hexamethylene diisocyanate (Bayhydur®) or mixtures thereof.

The above-mentioned compounds expressly encompass the different isomers, if present, alone or in combination as well as the corresponding derivatives. For instance, methylenebis(cyclohexyl isocyanate) (H12MDI) encompasses 4,4′-methylenebis(cyclohexyl isocyanate), 2,4′-methylenebis(cyclohexyl isocyanate) and/or 2,2′-methylenebis(cyclohexyl isocyanate) or the aliphatic polyisocyanate adducts such as Takenate™ D-110N and D-120N by Mitsui Chemicals.

Other particularly preferred monomeric isocyanate compounds are: diisocyanates such as 1,4-diisocyanatobutane, 1,6-diisocyanatohexane, 1,5-diisocyanato-2,2-dimethylpentane, 2,2,4- and 2,4,4-trimethyl-1,6-diisocyanatohexane, 1,10-diisocyanatodecane, 1,3- and 1,4-Diisocyanatocyclohexane, 1-isocyanato-3,3,5-trimethyl-5-isocyanatomethylcyclohexane (isophorone diisocyanate), 4,4′-diisocyanatodicyclohexylmethane, 2,4- and 2,6-diisocyanatomethylcyclohexane and mixtures thereof. Preferred polyisocyanates within the scope of the present invention are 1,5-pentamethylene diisocyanate (PDI) (Stabio™) and/or 1,3-bis(isocyanatomethyl)cyclohexane (Takenate™ 600) both from Mitsui Chemical, allowing the formation of highly stable and good performing core-shell microcapsules. Also preferred are adducts such as Takenate™ D-110N and D-120N by Mitsui Chemicals.

In another variant, the combination of at least two different polyisocyanates is preferred. It was observed that a particularly stable and better, i.e., more densely branched crosslinking is achieved within the capsule shell if different polyisocyanates were used as a mixture. Thereby, in such mixtures of isocyanates the polyisocyanates can either be all linear aliphatic, all branched aliphatic, all cycloaliphatic or all aromatic or a combination of, for example, at least one aromatic polyisocyanate and at least one aliphatic polyisocyanate. Generally, all possible combinations of said substances are suitable for the purpose of the present invention and for achieving a denser crosslinking resulting in more stable and efficient encapsulations while simultaneously allowing for improved performances.

However, preferably at least one of the one or more polyisocyanates having at least two isocyanate groups is aliphatic, and even more preferred cycloaliphatic. Even more preferred, the polyisocyanate component of the oil phase is a mixture of two or more aliphatic and/or aromatic polyisocyanates each having at least two isocyanate groups. Consequently, the isocyanate component can comprise two or more different aliphatic polyisocyanates, two or more different aromatic polyisocyanates or a mixture of at least one aliphatic and at least one aromatic polyisocyanate. It was surprisingly found that such mixtures of two or more polyisocyanates result in more stable and better performing encapsulations, especially when used as fragrance microcapsules. Preferably, at least one of the polyisocyanates used in such a mixture is aliphatic, i.e., linear aliphatic, branched aliphatic or cycloaliphatic as these allow the formation of highly stable microcapsule, especially in consumer product formulation and under mechanical stress (such as in a washing machine or drier) or heat, and which simultaneously show excellent release properties.

Moreover, it is known that aliphatic isocyanates show a lower toxicity compared to the aromatic polyisocyanates, thus the resulting structures being more biocompatible than, for example aromatic isocyanate-based polyurethanes.

Therefore, in a preferred embodiment of the present invention at least one aliphatic polyisocyanate is used for the preparation of microcapsules according to the invention and/or the microcapsules according to the first aspect comprise the reaction product of at least one aliphatic polyisocyanate with at least one saccharide and/or at least one aminosaccharide having less than 20 monomeric units, respectively, and thus in the microcapsules according to the invention at least one of the one or more polyisocyanates having at least two isocyanate groups comprises an aliphatic structure.

In one variant thereof, preferably only aliphatic polyisocyanates are used, i.e., a mixture of two or more aliphatic isocyanates which can be for example linear, branched, or cycloaliphatic.

As indicated above, in another variant, preferably the capsules are prepared using a mixture of at least one aliphatic polyisocyanate and at least one aromatic polyisocyanate, i.e., the core-shell microcapsules according to the first aspect comprise the reaction product of at least one polyisocyanate with at least one saccharide and/or at least one aminosaccharide having less than 20 monomeric units, wherein at least one of the one or more polyisocyanates having at least two isocyanate groups comprises an aliphatic structure, allowing for the formation of highly effective microcapsules.

Preferably, the molar or weight ratio, and preferably the molar ratio, of the at least one aliphatic polyisocyanate to the at least one aromatic polyisocyanate is ranging from 85:15, and even more preferred from 90:10 to 99:1, for microcapsules being preferably based on or comprising aminosaccharides having preferably more than one monomeric unit allowing for the formation of highly stable but at the same time excellently performing capsules (in terms of the targeted release behavior), showing an increased stability within product formulation even after four-week ageing in said product formulation (see Example 11).

The combination of different polymerizable polyisocyanates therefore results in particularly stable capsule shells or capsule walls, which in turn is reflected in a better encapsulation of the active ingredient and reduced losses during storage but simultaneously allowing for a better performance (i.e., excellent odorant release behavior with high intensities) of the capsules, for example in the area of fragrance or odorant encapsulation. Accordingly, in the present invention the combination of at least two different polymerizable isocyanates is generally preferred. Even more preferred, at least one of said isocyanates is aliphatic in nature irrespective of the (amino)saccharide component.

Therefore, preferably the capsule wall is based on the reaction of at least one aliphatic polyisocyanate having at least two isocyanate groups. Consequently, in step (a) of the process described above, preferably an oil phase comprising at least one aliphatic and optionally at least one aromatic polyisocyanate having at least two isocyanate groups, and preferably at least one aliphatic polyisocyanate and one or more preferably hydrophobic active ingredients is provided.

It should be noted that the terms “polyisocyanate”, “isocyanate” and “isocyanate component”, “isocyanate compound” or “polyisocyanate component” are used synonymously throughout the present disclosure. The same is true for the term “(poly)isothiocyanate”.

In a further embodiment, mixtures of isocyanate and isothiocyanate compounds can be used in the oil phase.

The proportion of the isocyanate component to the total oil phase is at least 0.2% by weight and preferably at least 0.5% by weight or particularly preferable at least 1% by weight of the oil phase. Thereby, the upper limit is dependent on the amount of functional —NCO, —OH, —NH₂ and —NH— groups so that no free isocyanate groups are remaining. However, preferably the upper limit is 10% by weight.

Based on the present invention, it is possible to produce microcapsules in which the absolute isocyanate content amounts to only a fraction of the total capsule containing the active ingredient(s), due to the low content of the isocyanate component. Thus, with the process described herein, it was possible to produce efficient microcapsules with a significantly reduced isocyanate content. Despite the isocyanate content being low, it was possible to obtain microcapsules which are highly stable and simultaneously show excellent release properties. In addition, due to the low isocyanate content concerns in view of health and environmental aspects could be considerably reduced. Additionally, based on the reduced isocyanate content the crosslink density of the shell wall could be reduced, without negatively influencing the stability and/or performance of the inventive microcapsules. Consequently, the overall isocyanate content is low relative to the total capsule weight.

In the process for the production of biobased microcapsules according to the invention, the at least one polymerizable isocyanate is preferably first dissolved in a hydrophobic medium together with the active ingredient(s) to be encapsulated. Preferably, the active ingredient as such is serving as the hydrophobic medium in which the isocyanate compound is dissolved, e.g., a perfume oil or fragrance substance or mixture, i.e., a substance or substance mixture having an olfactorily perceptible odor. In the context of the present description, the active ingredient to be encapsulated is thus preferably a hydrophobic active ingredient. The choice of such an active ingredient ensures that the material to be encapsulated is in the oil phase and does not mix with the external aqueous phase. Consequently, the hydrophobic ingredient forms the dispersed phase and correspondingly the aqueous phase is the continuous phase. This ensures that the hydrophobic active ingredient is actually effectively enclosed inside the microcapsule shell as the core material.

Suitable oil components for this purpose are specified in more detail below:

The oil phase may consist of the isocyanate component as defined above itself and the at least one active ingredient, however, it additionally can contain one or more oil components as a solvent having a c Log P (octanol/water partition coefficient) value being greater than 2, and preferably greater than 3 or even more preferred greater than 4, such as for example:
(i) linear or branched saturated paraffins (mineral oils) having 15 or more C-atoms, in particular having 18 to 45 C-atoms;
(ii) esters having 12 or more C-atoms of linear or branched fatty acids having 6 to 30 C-atoms and linear or branched, saturated or unsaturated mono-, di- or triols having 3 to 30 C-atoms, wherein these esters do not have free hydroxyl groups;
(iii) esters of benzoic acid and linear or branched, saturated or unsaturated monoalcohols having 8 to 20 C-atoms;
(iv) monoesters or diesters of alcohols having 3 to 30 C-atoms and naphthalenemonocarboxylic or dicarboxylic acids; especially naphthalenemonocarboxylic acid C₆-C₁₈ esters and naphthalenedicarboxylic acid di-C₆-C₁₈ esters;
(v) linear or branched, saturated or unsaturated di-C₆-C₁₈-alkyl ethers;
(vi) silicone oils;
(vii) 2-alkyl-1-alkanols of the following formula:

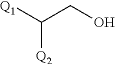

wherein Q₁ is a linear or branched alkyl radical having 6 to 24 C-atoms and Q₂ is a linear or branched alkyl radical having 4 to 16 C-atoms.

Thereby, the c Log P value (partition coefficient) is defined as the ratio of concentrations of a compound in a mixture of two immiscible solvents at equilibrium representing a measure of the lipophilicity or hydrophobicity of a specific substance.

An oil phase or oil component in the narrower (and preferred) sense of the present invention can encompass the following groups of substances:
(i) linear or branched, saturated paraffins having 20 to 32 C-atoms;
(ii) esters having at least 14 C-atoms of linear or branched, saturated fatty acids having 8 to 24 C-atoms and linear or branched, saturated or unsaturated mono-, di- or triols having 3 to 24 C-atoms, wherein these esters do not contain free hydroxyl groups;
(iii) esters of benzoic acid and linear or branched, saturated monoalcohols having 10 to 18 C-atoms;
(iv) alkylenediol dicaprylate caprates, especially propylenediol dicaprylate caprate;
(v) linear or branched, saturated di-C₆-C₁₈-alkyl ethers, especially (straight-chain) di-C₆-C₁₂-alkyl ethers;
(vi) silicone oils from the group of the cyclotrisiloxanes, cyclopentasiloxanes, dimethylpolysiloxanes, diethylpolysiloxanes, methylphenylpolysiloxanes, diphenylpolysiloxanes and hybrid forms thereof;
(vii) 2-alkyl-1-alkanols having 12 to 32 C-atoms of the following formula:

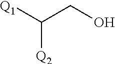

wherein Q₁ is a (preferably linear) alkyl radical having 6 to 18 C-atoms and Q₂ is a (preferably linear) alkyl radical having 4 to 16 C-atoms.

An oil phase in the narrowest (and most preferred) sense of the present invention can encompass the following groups of substances:
(i) linear or branched, saturated paraffins having 20 to 32 C-atoms such as isoeicosane or squalane;
(ii) esters having at least 16 C-atoms of linear or branched, saturated fatty acids having 8 to 18 C-atoms and linear or branched, saturated mono-, di- or triols having 3 to 18 C-atoms, these esters containing no free hydroxyl groups;
(iii) esters of benzoic acid and linear or branched, saturated monoalcohols having 12 to 15 C-atoms, especially C₁₂-C₁₅-alkyl benzoates;
(iv) alkylenediol dicaprylate caprates especially propylenediol dicaprylate caprate;
(v) straight-chain di-C₆-C₁₀-alkyl ethers; especially di-n-octyl ether (dicaprylyl ether);
(vi) silicone oils from the group undecamethylcyclotrisiloxane, cyclomethicone, decamethylcyclopentasiloxane, dimethylpolysiloxanes, diethylpolysiloxanes, methylphenylpolysiloxanes and diphenylpolysiloxanes;
(vii) 2-alkyl-1-alkanols having 12 to 32 C-atoms of the following formula:

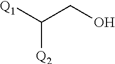

wherein Q₁ is a (preferably linear) alkyl radical having 6 to 18 C-atoms and Q₂ is a (preferably linear) alkyl radical having 4 to 16 C-atoms.

Particularly preferred components of the group of type (i) in the oil phase are as follows:
isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, 2-ethylhexyl isostearate, isotridecyl isononanoate, 2-ethylhexyl cocoate, caprylic/capric triglyceride, alkylenediol dicaprylate caprates, especially propylenediol dicapylate caprate, and also synthetic, semisynthetic and natural mixtures of such esters, e.g., jojoba oil.

Fatty acid triglycerides (oil components of type (i) in the oil phase) may also be in the form of, or in the form of a constituent of, synthetic, semisynthetic and/or natural oils, examples being olive oil, sunflower oil, soya oil, peanut oil, rapeseed oil, almond oil, palm oil, coconut oil, palm kernel oil and mixtures thereof.

Particularly preferred oil components of type (vii) in the oil phase are as follows: 2-butyl-1-octanol, 2-hexyl-1-decanol, 2-octyl-1-dodecanol, 2-decyltetradecanol, 2-dodecyl-1-hexadecanol and 2-tetradecyl-1-octadecanol.

Particularly preferred oil components in the oil phase are mixtures comprising C₁₂-C₁₅-alkyl benzoate and 2-ethylhexyl isostearate, mixtures comprising C₁₂-C₁₅-alkyl benzoate and isotridecyl isononanoate, mixtures comprising C₁₂-C₁₅-alkyl benzoate, 2-ethylhexyl isostearate and isotridecyl isononanoate, mixtures comprising cyclomethicone and isotridecyl isononanoate, and mixtures comprising cyclomethicone and 2-ethylhexyl isostearate.

Preferred oil bodies, used within the scope of the present invention, are, for example, Guerbet alcohols based on fatty alcohols having 6 to 18, preferably 8 to 10, carbon atoms, esters of linear C₆-C₂₂-fatty acids with linear or branched C₆-C₂₂-fatty alcohols or esters of branched C₆-C₁₃-carboxylic acids with linear or branched C₆-C₂₂-fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂-fatty acids with branched alcohols, in particular 2-ethylhexanol, esters of C₁₈-C₃₈-alkylhydroxy carboxylic acids with linear or branched C₆-C₂₂-fatty alcohols, in particular Dioctyl Malate, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as, for example, dicaprylyl carbonate (Cetiol® CC), Guerbet carbonates, based on fatty alcohols having 6 to 18, preferably 8 to 10, carbon atoms, esters of benzoic acid with linear and/or branched C₆-C₂₂-alcohols (e.g., Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicone methicone grades, etc.) and/or aliphatic or naphthenic hydrocarbons, such as, for example, squalane, squalene or dialkylcyclohexanes. The most preferred oil components are triglycerides, particularly those of natural origin.

In a preferred embodiment the microcapsules of the present invention are loaded with one or more (hydrophobic) active ingredients, such as for example fragrances or perfume oils. For certain applications, also other additives can be used as indicated above. With regard to fragrances and perfume oils the inventive polyurethane and/or polyurea-based capsules allow loading high amounts of up to 80% by weight calculated based on the total capsule weight. The actives are preferably incorporated into the oil phase, however depending on their polarity they can also be incorporated into the first aqueous phase.

The term “active substance”, as used in the present invention, is to be understood to mean a substance or ingredient pertaining to a certain effect, for example a fragrance, an aroma, a dye, a pharmaceutical drug, a pesticide, etc. The preferably hydrophobic active substance is either part of the oil phase, i.e., with another component constituting the oil phase, or itself constitutes the oil component of the oil phase as used herein. Preferably, the hydrophobic substance and the polyisocyanate component constitute the oil phase. Fragrances and/or perfumes as used herein refer to both, single fragrance substances or perfume substances (also called odorous substances or fragrant substances), i.e., chemical compounds having a smell or odor and thus all natural and synthetic substances that impart an olfactorily perceptible odor, as well as compositions of one or more fragrance substances or perfume substances, i.e., mixtures of the aforementioned compounds or mixtures comprising said compounds.

The lists of these active substances and other ingredients are non-limiting and may include further active ingredients and other ingredients that are not elucidated further below.

Suitable fragrances and/or perfume oils are for example single odorous substances or mixtures of natural and synthetic odorous substances. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.-butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, chamomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

However, preferably the perfume substance or aroma substance used for encapsulation is a compound which is used for the primary purpose of conferring or modifying an odor or flavor. Preferably, these substances or mixtures of substances are able to impart or modify the odor or flavor of a composition in a positive or pleasant way. For the purposes of the present invention, the terms “perfume oil” or “aroma” include a combination of perfuming or flavoring ingredients for modifying or imparting an odor or flavor.

Besides or in addition to the fragrances/perfume substances and perfume oils exemplarily listed above, the at least one hydrophobic active substance can be preferably selected from the broader group consisting of aroma substances, aromas, agrochemicals, cosmetically active ingredients such as active skin-product ingredients like skin moisturizing agents, skin or hair conditioning agents, skin lightening agents, anti-acne agents and the like, active pharmaceutical ingredients, UV-active substances, optical brighteners, bodying agents, drape and form control agents, smoothness agents, static control agents, wrinkle control agents, sanitizing agents, disinfecting agents, germ control agents, mold control agents, mildew control agents, antiviral agents, antimicrobials, drying agents, stain resistance agents, soil release agents, malodor control agents, fabric freshening agents, dyes and dye fixatives, color maintenance agents, color restoring/rejuvenating agents, anti-fading agents, anti-abrasion agents, wear resistance agents, fabric integrity agents, anti-wear agents, rinsing aids, UV-protection agents, sun fade inhibitors, insect repellents, anti-allergenic agents, flame retardants, water-proofing agents, fabric softening agents, shrinkage resistance agents, stretch resistance agents, fluorescent paints, solvents, waxes, silicone oils, lubricants, cooling agents, TRPV-modulators (such as TRPV1 and TRPV2-modulators), impregnating agents, dirt-repellent agents, friction-reducing agents, as well as mixtures of the above-mentioned active ingredients.

Suitable cooling agents are known in the art and are, for example, menthol-based cooling agents such as Frescolat® and the like. Preferred individual cooling agents for use within the framework of the present invention are listed below. The person skilled in the art can add many other cooling agents to this list; the cooling agents listed can also be used in combination with one another, which are preferably selected here from the following list: menthol and menthol derivatives (for example L-menthol, D-menthol, racemic menthol, isomenthol, neoisomenthol, neomenthol) menthylethers (for example (l-menthoxy)-1,2-propanediol, (l-menthoxy)-2-methyl-1,2-propanediol, l-menthyl-methylether), menthone glyceryl acetal, menthone glyceryl ketal or mixtures of both, menthylesters (for example menthylformiate, menthylacetate, menthylisobutyrate, menthyhydroxyisobutyrat, menthyllactates, L-menthyl-L-lactate, L-menthyl-D-lactate, menthyl-(2-methoxy)acetate, menthyl-(2-methoxyethoxy)acetate, menthylpyroglutamate), menthylcarbonates (for example menthylpropyleneglycolcarbonate, menthylethyleneglycolcarbonate, menthylglycerolcarbonate or mixtures thereof), the semi-esters of menthols with a dicarboxylic acid or derivatives thereof (for example mono-menthylsuccinate, monomenthylglutarate, mono-menthylmalonate, O-menthyl succinic acid ester-N,N-(dimethyl)amide, O-menthyl succinic acid ester amide), menthanecarboxylic acid amides (in this case preferably menthanecarboxylic acid-N-ethylamide [WS3] or Nα-(menthanecarbonyl)glycinethylester [WS5], menthanecarboxylic acid-N-(4-cyanophenyl)amide or menthanecarboxylic acid-N-(4-cyanomethylphenyl)amide, menthanecarboxylic acid-N-(alkoxyalkyl)amides), menthone and menthone derivatives (for example L-menthone glycerol ketal), 2,3-dimethyl-2-(2-propyl)-butyric acid derivatives (for example 2,3-dimethyl-2-(2-propyl)-butyric acid-N-methylamide [WS23]), isopulegol or its esters (l-(−)-isopulegol, l-(−)-isopulegolacetate), menthane derivatives (for example p-menthane-3,8-diol), cubebol or synthetic or natural mixtures, containing cubebol, pyrrolidone derivatives of cycloalkyldione derivatives (for example 3-methyl-2(1-pyrrolidinyl)-2-cyclopentene-1-one) or tetrahydropyrimidine-2-one (for example iciline or related compounds, as described in WO 2004/026840), further carboxamides (for example N-(2-(pyridin-2-yl)ethyl)-3-p-menthanecarboxamide or related compounds), (1R,2S,5R)—N-(4-Methoxyphenyl)-5-methyl-2-(1-isopropyl)cyclohexane-carboxamide [WS12], oxamates and [(1R,2S,5R)-2-isopropyl-5-methyl-cyclohexyl] 2-(ethylamino)-2-oxo-acetate (X Cool). Cooling agents which are preferred due to their particular synergistic effect are L-menthol, D-menthol, racemic menthol, menthone glycerol acetal (trade name: Frescolat® MGA), menthyl lactate (preferably L-menthyl lactate, in particular L-menthyl L-lactate (trade name: Frescolat® ML)), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate and isopulegol. Particularly preferred cooling agents are L-menthol, racemic menthol, menthone glycerol acetal (trade name: Frescolat® MGA), menthyl lactate (preferably L-menthyl lactate, in particular L-menthyl L-lactate (trade name: Frescolat® ML)), 3-menthoxypropane-1,2-diol, 2-hydroxyethyl menthyl carbonate and 2-hydroxypropyl menthyl carbonate.

In addition, TRPV1 and TRPV2 modulators such as capsaicin and other TRPV1 and 2-responsive substances known in the art can effectively be encapsulated by the inventive core-shell microcapsules. A preferred capsule according to the present invention comprises, for example, one or more TRPV1 antagonists. Suitable compounds which reduce the hypersensitivity of skin nerves based on their action as TRPV1 antagonists, encompass e.g., trans-4-tert.-butyl cyclohexanol, or indirect modulators of TRPV1 by an activation of the μ-receptor, e.g., acetyl tetrapeptide-15.

The cosmetically or pharmaceutically active ingredients and/or adjuvants and/or additives or auxiliaries, which can suitably be used are amongst others for example abrasives, antiacne agents, agents against ageing of the skin, anti-cellulitis agents, anti-dandruff agents, anti-inflammatory agents, anti-microbial agents, irritation-preventing agents, irritation-inhibiting agents, antioxidants, astringents, odor absorbers, perspiration-inhibiting agents, antiseptic agents, anti-statics, binders, buffers, carrier materials, chelating agents, cell stimulants, cleansing agents, depilatory agents, surface-active substances, deodorizing agents, antiperspirants, softeners, emulsifiers, enzymes, enzyme inhibitors, essential oils, fibers, film-forming agents, fixatives, foam-forming agents, foam stabilizers, substances for preventing foaming, foam boosters, gelling agents, gel-forming agents, hair care agents, hair-setting agents, hair-straightening agents, moisture-donating agents, moisturizing substances, moisture-retaining substances, bleaching agents, strengthening agents, stain-removing agents, lubricants, moisturizing creams, ointments, opacifying agents, plasticizing agents, covering agents, polish, preservatives, gloss agents, green and synthetic polymers, powders, proteins, re-oiling agents, abrading agents, silicones, skin-soothing agents, skin-cleansing agents, skin care agents, skin-healing agents, skin-lightening agents, skin-protecting agents, skin-softening agents, hair promotion agents, cooling agents, skin-cooling agents, warming agents, skin-warming agents, stabilizers, surfactants, UV-absorbing agents, UV-filters, primary sun protection factors, secondary sun protection factors, detergents, fabric conditioning agents, suspending agents, skin-tanning agents, actives modulating skin or hair pigmentation, matrix-metalloproteinase inhibitors, skin moisturizing agents, glycosaminoglycan stimulators, TRPV1 antagonists, desquamating agents, or fat enhancing agents, hair growth activators or inhibitors, thickeners, rheology additives, vitamins, oils, waxes, pearlizing waxes, fats, phospholipids, saturated fatty acids, mono- or polyunsaturated fatty acids, α-hydroxy acids, polyhydroxy fatty acids, liquefiers, dyestuffs, color-protecting agents, pigments, anti-corrosives, fragrances or perfume oils, odoriferous substances, polyols, electrolytes, organic solvents, and mixtures of two or more of the aforementioned substances, as further described below. As active skin-product ingredients, for example, humectants, biogenic agents, antioxidants etc. can be used.

Suitable soil release polymers, which are also referred to as “antiredeposition agents”, are for example non-ionic cellulose ethers such as methyl cellulose and methylhydroxypropyl cellulose or hydroxypropylcellulose (HPC) (Klucel®) having a proportion of methoxy groups of 15% to 30% by weight and of hydroxypropyl groups of 1% to 15% by weight, based in each case on the non-ionic cellulose ethers, and also the polymers, known from the prior art, of phthalic acid and/or terephthalic acid or of derivatives thereof, especially polymers of ethylene terephthalates and/or polyethylene and/or polypropylene glycol terephthalates or anionically and/or non-ionically modified derivatives of these. Suitable derivatives include the sulphonated derivatives of phthalic acid and terephthalic acid polymers.

Optical brighteners (so-called “whitening agents”) can be incorporated into the capsules according to the invention in order to eliminate greying and yellowing of the treated textile surfaces. These substances are absorbed by the fibers, brightening them and simulating a bleaching effect, by converting invisible ultraviolet radiation into visible longer-wavelength light, wherein the ultraviolet light absorbed from sunlight is given off as a slightly blue fluorescence, combining with the yellow color of the greyed or yellowed laundry to give a pure white color. Suitable compounds are derived for example from the substance classes of the 4,4′-diamino-2,2′-stilbene disulfonic acids (flavonic acids), 4,4′-distyryl-biphenylenes, methyl umbelliferones, coumarins, dihydroquinolinones, 1,3-diarylpyrazolines, naphthalic acid imides, benzoxazole, benzisoxazole and benzimidazole systems, and heterocycle-substituted pyrene derivatives.

Greying inhibitors have the task of keeping the dirt detached from the fibers suspended in the liquor and hence of preventing reattachment of the dirt. Suitable for this purpose are water-soluble colloids, usually organic in nature, for example size, gelatin, salts of ethersulphonic acids of starch or of cellulose or salts of acidic sulphuric acid esters of cellulose or of starch. Also suitable for this purpose are water-soluble polyamides containing acidic groups. In addition, it is possible to use soluble starch preparations and starch products other than those specified above, for example degraded starch, aldehyde starches etc. It is also possible to use polyvinylpyrrolidone. However, preference is given to using cellulose ethers such as carboxymethyl cellulose (sodium salt), methyl cellulose, hydroxyalkyl cellulose and mixed ethers such as methylhydroxyethyl cellulose, methylhydroxypropyl cellulose, methylcarboxymethyl cellulose and mixtures thereof.

Since textile fabrics, especially made of rayon, cellulose, cotton and mixtures thereof, can have a tendency to crease because the individual fibers are sensitive to bending, folding, pressing and squashing transverse to the fiber direction, the microcapsules according to the invention may contain synthetic anti-crease agents. These include, for example, synthetic products based on fatty acids, fatty acid esters, fatty acid amides, fatty acid alkylol esters, fatty acid alkylolamides or fatty alcohols, which have usually been reacted with ethylene oxide, or products based on lecithin or modified phosphoric acid esters.

Increased wearing comfort can be achieved by the additional use of antistatics. Antistatics increase the surface conductivity, thus allowing built-up charges to be more easily discharged. As a rule, antistatics are substances with at least one hydrophilic molecular ligand that provide a more or less hygroscopic film on surfaces. These antistatics, which are usually surface-active, can be divided into nitrogen-containing (amines, amides, quaternary ammonium compounds), phosphorus-containing (phosphoric acid esters) and sulfur-containing (alkyl sulfonates, alkyl sulfates) antistatics. Lauryl-(or stearyl)dimethylbenzyl ammonium chlorides are suitable as antistatics for textile surfaces or as additives to detergents and cleaning agents, wherein an additional conditioning effect is also achieved.

Humectants serve to regulate moisture on the skin. Humectants preferred according to the invention include amino acids, pyrrolidone carboxylic acid, lactic acid and salts thereof, lactitol, urea and urea derivatives, uric acid, glucosamine, creatinine, cleavage products of collagen, chitosan or chitosan salts/derivatives, and in particular polyols and polyol derivatives (e.g., glycerol, diglycerol, triglycerol, ethylene glycol, propylene glycol, butylene glycol, erythritol, 1,2,6-hexane triol, polyethylene glycols such as PEG-4, PEG-6, PEG-7, PEG-8, PEG-9, PEG-10, PEG-12, PEG-14, PEG-16, PEG-18, PEG-20), sugar and sugar derivatives (including fructose, glucose, maltose, maltitol, mannitol, inositol, sorbitol, sorbitol silane diol, sucrose, trehalose, xylose, xylitol, glucuronic acid and salts thereof), ethoxylated sorbitol (Sorbeth-6, Sorbeth-20, Sorbeth-30, Sorbeth-40), honey and hardened honey, hardened starch hydrolysates and mixtures of hardened wheat protein and PEG-20-acetate copolymer. Preferred according to the invention as suitable humectants are glycerol, diglycerol, triglycerol and butylene glycol.

Biogenic agents are understood for example to refer to tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts such as e.g., prune extract, Bambara nut extract and vitamin complexes.

In order to prevent undesired changes in the treated textile surfaces caused by the action of oxygen and other oxidative processes, the macroemulsion can comprise antioxidants. Examples of compounds of this class include amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, -carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cysteamine and glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximine, homocysteine sulfoximine, buthionine sulfone, penta-, hexa-, heptathionine sulfoximine) in very low tolerable dosages (e.g. pmol to μmol/kg), further (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin-A-palmitate) and coniferyl benzoate from benzoic resin, rutinic acid and derivatives thereof, α-glycosylrutin, ferulic acid, furfurylidene glucitol, carnosine, butylhydroxytoluene, butylhydroxyanisole, nordihydroguaiaretic acid, trihydroxybutyrophenone, uric acid and derivatives thereof, mannose and derivatives thereof, superoxide dismutase, zinc and derivatives thereof (e.g. ZnO, ZnSO₄) selenium and derivatives thereof (e.g. selenium methionine), stilbenes and derivatives thereof (e.g. stilbene oxide, trans-stilbene oxide) and suitable derivatives according to the invention of the above-mentioned agents (salts, esters, ethers, sugars, nucleotides, nucleosides, peptides and lipids).

Suitable anti-dandruff agents are piroctone olamine (1-hydroxy-4-methyl-6-(2,4,4-trimythylpentyl)-2-(1H)-pyridinone monoethanolamine salt), Baypival® (climbazole), Ketoconazol®, (4-acetyl-1{-4-[2-(2,4-dichlorphenyl) r-2-(1H-imidazol-1-ylmethyl)-1,3-dioxylan-c-4-ylmethoxyphenyl}piperazine, ketoconazole, elubiol, selenium disulfide, colloidal sulfur, sulfur polyethylene glycol sorbitan monooleate, sulfur ricinol polyethoxylate, sulfur-tar distillates, salicylic acid (or in combination with hexachlorophene), undecylenic acid monoethanolamide sulfosuccinate Na-salt, Lamepon® UD (protein-undecylenic acid condensate), zinc pyrithione, aluminum pyrithione and magnesium pyrithione/dipyrithione-magnesium sulfate.

Cosmetic deodorants (deodorizing agents) counteract body odors or mask or eliminate them. Body odors occur due to the effect of skin bacteria on apocrine perspiration, wherein unpleasant-smelling decomposition products are formed. Accordingly, deodorants comprise agents that function as antimicrobial agents, enzyme inhibitors, odor absorbers, and odor-masking agents.

As antimicrobial agents, all substances active against grampositive bacteria are generally suitable, such as e.g. 4-hydroxybenzoic acid and salts and esters thereof, N-(4-chlorophenyl)-N′(3,4-dichlorophenyl)urea, 2,4,4′-trichloro-2′-hydroxy-di-phenylether (triclosan), 4-chloro-3,5-dimethylphenol, 2,2′-methylene-bis(6-bromo-4-chlorophenol), 3-methyl-4-(1-methylethyl)-phenol, 2-benzyl-4-chlorophenol, 3-(4-chlorophenoxy)-1,2-propanediol, 3-iodo-2-propinylbutylcarbamate, chlorohexidine, 3,4,4′-trichlorocarbanilide (TTC), antibacterial fragrances, thymol, thyme oil, eugenol, clove oil, menthol, mint oil, farnesol, phenoxyethanol, glycerol monocaprinate, glycerol monocaprylate, glycerol monolaurate (GML), diglycerol monocaprinate (DMC), salicylic acid-N-alkylamides such as e.g. salicylic acid-n-octylamide or salicylic acid-n-decylamide.

Examples of suitable enzyme inhibitors are esterase inhibitors. These are preferably trialkyl citrates such as trimethyl citrate, tripropyl citrate, triisopropyl citrate, tributyl citrate and in particular triethyl citrate (Hydagen® CAT). These substances inhibit enzyme activity and thus reduce the formation of odors. Further substances suitable as esterase inhibitors are sterol sulfates or phosphates, such as e.g. lanosterol, cholesterol, campesterol, stigmasteoilrol and sitosterol sulfates or phosphates, dicarboxylic acids and esters thereof, such as e.g. glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof such as e.g. citric acid, malic acid, tartaric acid or tartaric acid diethyl ester, and zinc glycinate.

Suitable as odor absorbers are substances that are capable of absorbing odor-causing compounds and largely retaining them. They reduce the partial pressure of the individual components and thus reduce their rate of diffusion. It is important in this case that perfumes as specified herein must remain unaffected. Odor absorbers have no effect against bacteria. They comprise for example as the main component a complex zinc salt of ricinoleic acid or special, largely odor-neutral substances that are known to the person having ordinary skill in the art as “fixators,” such as e.g., extracts of labdanum or styrax or certain abietic acid derivatives. Fragrances or perfume oils that, in addition to their function as odor-masking agents, provide deodorants with their respective scents act as odor-masking agents. Examples of perfume oils that can be mentioned are mixtures of natural and synthetic fragrances. Natural fragrances are for example extracts of flowers, stems and leaves, fruits, fruit peels, roots, woods, hers and grasses, needles and branches, and resins and balsams. Also suitable are animal raw materials such as e.g., civet and castoreum. Typical synthetic fragrance compounds are products of the ester, ether, aldehyde, ketone, alcohol and hydrocarbon types. Fragrance compounds of the ester type are e.g., benzyl acetate, p-tert.-butylcyclohexyl acetate, linalyl acetate, phenethyl acetate, linalyl benzoate, benzyl formiate, allylcyclohexyl propionate, styrallyl propionate and benzyl salicylate. Examples of ethers include benzyl ethyl ether, and examples of aldehydes include linear alkanals with 8 to 18 carbon atoms, citral, citronellal, citronellyl oxyacetaldehyde, cyclamen aldehyde, hydroxycitronellal, lilial and bourgeonal, examples of ketones include the ionones and methyl cedryl ketone, alcohols include anethol, citronellol, eugenol, isoeugenol, geraniol, linalool, phenethyl alcohol and terpineol, and hydrocarbons include mainly the terpenes and balsams. Preferred, however, are mixtures of various fragrances that together produce a pleasant scent. Essential oils of low volatility, which are usually used as flavoring components, are also suitable as perfume oils, e.g., sage oil, chamomile oil, clove oil, Melissa oil, mint oil, cinnamon leaf oil, linden flower oil, juniper berry oil, vetiver oil, olibanum oil, galbanum oil, labdanum oil and lavandin oil. Preferred are bergamot oil, dihydromyrcenol, lilial, lyral, citronellol, phenethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-Damascone, geranium oil bourbon, cyclohexyl salicylate, Vertofix Coeur, iso-E-super, Fixolide NP, evernyl, iraldein gamma, phenylacetic acid, geranyl acetate, benzyl acetate, rose oxide, Romilat, Irotyl and Floramat are used alone or in mixtures.

Antiperspirants (antiperspirant agents) reduce the formation of perspiration by affecting the activity of the eccrine sweat glands, thus counteracting underarm wetness and body odor. Aqueous or anhydrous formulations of antiperspirants typically comprise the following ingredients: astringent agents, oil components, nonionic emulsifiers, coemulsifiers, bodying agents, excipients such as e.g., thickeners or complexing agents and/or non-aqueous solvents such as e.g., ethanol, propylene glycol and/or glycerol. Suitable as astringent antiperspirant agents are primarily salts of aluminum, zirconium or zinc. Examples of such suitable antihydrotically active agents are e.g., aluminum chloride, aluminum chlorohydrate, aluminum dichlorohydrate, aluminum sesquichlorohydrate and complex compounds thereof, e.g., with propylene glycol-1,2-aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, e.g., with amino acids such as glycine. In addition, antiperspirants may comprise common oil-soluble and water-soluble excipients in small amounts. Such oil-soluble excipients can for example include: anti-inflammatory, skin-protective, or fragrant essential oils, synthetic skin-protective agents and/or oil-soluble perfume oils.

The capsules according to the invention can comprise antimicrobial agents in order to combat microorganisms. In this case, a distinction is made depending on the antimicrobial spectrum and mechanism of action between bacteriostates and bactericides, fungistates and fungicides, etc. Examples of important substances from these groups are benzalkonium chlorides, alkylaryl sulfonates, halophenols and phenylmercuric acetates, wherein these compounds may also be dispensed with entirely in the detergents and cleaning agents according to the invention. As antimicrobial agents, all substances active against grampositive bacteria are generally suitable, such as e.g. 4-hydroxybenzoic acid and salts and esters thereof, N-(4-chlorophenyl)-N′(3,4-dichlorophenyl)urea, 2,4,4′-trichloro-2′-hydroxy-di-phenylether (triclosan), 4-chloro-3,5-dimethylphenol, 2,2′-methylene-bis(6-bromo-4-chlorophenol), 3-methyl-4-(1-methylethyl)-phenol, 2-benzyl-4-chlorophenol, 3-(4-chlorophenoxy)-1,2-propanediol, 3-iodo-2-propinylbutylcarbamate, chlorohexidine, 3,4,4′-trichlorocarbanilide (TTC), antibacterial fragrances, thymol, thyme oil, eugenol, clove oil, menthol, mint oil, farnesol, phenoxyethanol, glycerol monocaprinate, glycerol monocaprylate, glycerol monolaurate (GML), diglycerol monocaprinate (DMC), salicylic acid-N-alkylamides such as e.g. salicylic acid-n-octylamide or salicylic acid-n-decylamide.

Among the compounds serving as bleaching agents that yield H₂O₂ in water, sodium perborate tetrahydrate and sodium perborate monohydrate are of particular importance. Further useful bleaching agents are for example sodium percarbonate, peroxypyrophosphates, citrate perhydrates and H₂O₂-yielding peracidic salts or peracids such as perbenzoates, peroxophthalates, diperazelaic acid, phthaloiminoperacid or diperdodecanedioc acid. Compounds that yield aliphatic peroxocarboxylic acids, preferably with 1 to 10 C-atoms, in particular 2 to 4 C-atoms, and/or optionally substituted perbenzoic acid under perhydrolysis conditions can be used as bleach activators. Suitable are substances bearing O- and/or N-acyl groups with the above-mentioned number of C-atoms and/or optionally substituted benzoyl groups. Preferred are polyacylated alkylene diamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoyl succinimide (NOSI), acylated phenol sulfonates, in particular N-nonanoyl or isononanoyl oxybenzene sulfonate (n- or iso-NOBS), carboxylic anhydrides, in particular phthalic acid anhydride, and acylated polyhydric alcohols, in particular triacetin, ethylene glycol diacetate and 2,5-diacetoxy-2,5-dihydrofuran. In addition to or instead of the conventional bleach activators, so-called bleach catalysts can also be incorporated into the textile treatment agents via the inventive capsules. These substances are bleach-enhancing transition metal salts or transition metal complexes such as e.g., Mn-, Fe-, Co-, R- or Mo-salt complexes or -carbonyl complexes. Mn-, Fe-, Co-, Ru-, Mo-, Ti-, V- and Cu-complexes with nitrogen-containing tripod ligands and Co-, Fe-, Cu- and Ru-ammine complexes can also be used as bleach catalysts.

The capsules according to the invention can also comprise preservatives. Examples are sorbic acid and salts thereof, benzoic acid and salts thereof, salicylic acid and salts thereof, phenoxyethanol, 3-iodo-2-propynyl butylcarbamate, sodium N-(hydroxymethyl)glycinate, biphenyl-2-ol and mixtures thereof. A suitable preservative is the solvent-free, aqueous combination of diazolidinyl urea, sodium benzoate and potassium sorbate (obtainable as Euxyl® K 500 from Schülke and Mayr), which may be used in a pH range of up to 7. Preservatives based on organic acids and/or salts thereof are particularly suitable for preserving the skin-friendly detergents and capsules according to the invention.

Silicone derivatives, for example, may be used in textile treatment in order to improve the rewettability and facilitate ironing of treated textile surfaces. These further improve the rinsing behavior of the detergents and cleaning agents by means of their foam-inhibiting properties. Preferred silicone derivatives are for example polydialkyl or alkylaryl siloxanes in which the alkyl groups have one to five C-atoms and are completely or partially fluorinated. Preferred silicones are polydimethylsiloxanes, which can optionally be derivatized and are then aminofunctional or quaternized or have Si—OH, Si—H and/or Si—Cl bonds. The viscosities of the preferred silicones at 25° C. are in the range of 100 mPas to 100 000 mPas.

The capsules according to the present invention can also comprise insect repellents. Suitable insect repellents are, for example, N, N-diethyl-m-toluamide, 1,2-pentanediol or ethyl butyl acetyl aminopropionate, 1-(1-methylpropoxycarbonyl)-2-(2-hydroxyethyl)piperidine, p-menthane-3,8-diol, 2-undecanone, as well as essential oils, such as citronella oil, lemongrass oil, lavender oil, neem oil and eucalyptus oil, in particular p-menthane-3,8-diol, essential oils, such as citronella oil, lemongrass oil, lavender oil, neem oil and eucalyptus oil.

The capsules according to the present invention can also comprise UV-absorbers that can be absorbed onto the treated textile surface and improve the light stability of the fibers. The term UV-light protection factors is understood for example to refer to organic substances that are liquid or crystalline at room temperature (light protection filters), which are capable of absorbing ultraviolet rays and giving off the absorbed energy in the form of longer-wavelength radiation, e.g., heat. The UV-light protection factors are ordinarily present in amounts of 0.1 wt.-% to 5 wt.-% and preferably 0.2 wt.-to 1 wt.-%. UVB-filters can be oil-soluble or water-soluble. Examples of oil-soluble substances include, for example: 3-benzylidene camphor or 3-benzylidene norcamphor and derivatives thereof, e.g. 3-(4-methylbenzylidene) camphor; 4-aminobenzoic acid derivatives, preferably 4-(dimethylamino)benzoic acid-2-ethyl-hexyl ester, 4-(dimethylamino)benzoic acid-2-octyl ester and 4-(dimethylamino)benzoic acid amyl ester; esters of cinnamic acid, preferably 4-methoxycinnamic acid-2-ethylhexyl ester, 4-methoxy-cinnamic acid propyl ester, 4-methoxycinnamic acid isoamyl ester, and 2-cyano-3,3-phenylcinnamic acid-2-ethylhexyl ester(octocrylene); esters of salicylic acid, preferably salicylic acid-2-ethylhexyl ester, salicylic acid-4-iso-propylbenzyl ester, and salicylic acid homomenthyl ester; derivatives of benzophenone, preferably 2-hydroxy-4-methoxybenzophenone, 2-hydroxy-4-methoxy-4′-methylbenzophenone, and 2,2′-dihydroxy-4-methoxybenzophenone; esters of benzyl malonic acid, preferably 4-methoxybenzyl malonic acid di-2-ethylhexyl-ester; triazine derivatives, such as e.g. 2,4,6-trianilino-(p-carbo-2′-ethyl-1′-hexyloxy)-1,3,5-triazine and octyl triazone or dioctyl butamidotriazone (Uvasorb® HEB); propane-1,3-diones, such as e.g. 1-(4-tert.-butylphenyl)-3-(4′methoxyphenyl)propane-1,3-dione; ketotricyclo(5.2.1.0)decane derivatives.

In particular, suitable UVA-filters include derivatives of benzoyl methane, such as e.g. 1-(4′-tert.-butylphenyl)-3-(4′-methoxyphenyl)propane-1,3-dione, 4-tert.-butyl-4′-methoxydibenzoyl methane (Parsol® 1789), 2-(4-diethylamino-2-hydroxybenzoyl)-benzoic acid hexyl ester (Uvinul® A Plus), 1-phenyl-3-(4′-isopropylphenyl)-propane-1,3-dione and enamine compounds. The UVA and UVB filters can of course also be used in mixtures. Particularly favorable combinations are composed of the derivatives of benzoylmethane, e.g., 4-tert.-butyl-4′-methoxydibenzoylmethane (Parsol® 1789) and 2-cyano-3,3-phenylcinnamic acid-2-ethyl-hexyl ester (octocrylene) in combination with esters of cinnamic acid, preferably 4-methoxycinnamic acid-2-ethylhexyl ester and/or 4-methoxycinnamic acid propyl ester and/or 4-methoxycinnamic acid isoamyl ester. Advantageously, these combinations are used together with water-soluble filters such as e.g., 2-phenylbenzimidazole-5-sulfonic acid and alkali, alkaline earth, ammonium, alkylammoium, alkanolammonium- and glucammonium salts thereof.

In addition to the above-mentioned soluble substances, insoluble light protection pigments, specifically finely dispersed metal oxides or salts, are also suitable for this purpose. Examples of suitable metal oxides are in particular zinc oxide and titanium dioxide, as well as oxides of iron, zirconium, silicon, manganese, aluminum and cerium and mixtures thereof. Silicates (talc), barium sulfate or zinc stearate may be used as salts. The oxides and salts are used in the form of pigments for skin care and skin protection emulsions and decorative cosmetics. In this case, the particles should have an average diameter of less than 100 nm, preferably 5 nm to 50 nm and in particular 15 nm to 30 nm. They can have a spherical shape, but particles may also be used that have an ellipsoid shape or another form deviating from the spherical shape. The pigments can also be present in surface-treated, i.e., hydrophilized or hydrophobized form. Typical examples are coated titanium dioxides, such as e.g., titanium dioxide T 805 (Degussa) or Eusolex® T2000, Eusolex® T, Eusolex® T-ECO, Eusolex® T-Aqua, Eusolex® T-45D (all Merck), Uvinul TiO2 (BASF). In this context, suitable hydrophobic coating agents are primarily silicones, specifically trialkoxyoctylsilanes or simethicones. In sun protection agents, so-called micro- or nanopigments are preferably used. Preferably, a micronized zinc oxide such as e.g., Z-COTE® or Z-COTE HP1® is used.

In addition, substances that complex heavy metals may be enclosed within the capsules according to the present invention. Suitable heavy metal complexing agents are for example the alkali salts of ethylene diamine tetra-acetic acid (EDTA) or nitrilotriacetic acid (NTA) and alkali metal salts of anionic polyelectrolytes such as polymaleates and polysulfonates. A preferred class of complexing agents are the phosphonates, which are included in preferred textile treatment agents in amounts of 0.01% to 2.5% by weight, preferably 0.02% to 2% by weight and in particular 0.03% to 1.5% by weight. Examples of these preferred compounds include in particular organophosphonates such as e.g. 1-hydroxyethane-1,1-diphosphonic acid (HEDP), aminotri(methylene phosphonic acid) (ATMP), diethylene triamine penta(methylene phosphonic acid) (DTPMP or DETPMP) and 2-phosphonobutane-1,2,4-tricarboxylic acid (PBS-AM), which are usually used in the form of their ammonium or alkali metal salts.

The preferably hydrophobic active substances specified above can be used either singularly or in a mixture of two or three or four or even more of the above substances.

According to a particular and preferred variant, the hydrophobic active substance comprises or consists of a perfume substance or aroma substance or at least one perfume oil or aroma as specified herein, i.e., a corresponding mixture of the afore-mentioned substances, if the capsules of the present invention are to be used for imparting, i.e., delivering or transferring, a fragrance or aroma, i.e., it is dispersed in the oil phase of the present invention.

Conceivable further ingredients generally include all compounds for which there exist applications from the field of microencapsulation. Such ingredients are well known in the art. However, active substances which are odorous such as fragrances and perfume oils are preferred, being both of synthetic and of natural origin. Preferred is a core-shell microcapsule according to the invention, wherein the core contains one or more odorous substances/fragrances as active agent(s) selected from the group consisting of extracts of natural raw materials such as essential oils, concretes, absolutes, resins, resinoids, balsams, non-primary or secondary alcohol-containing tinctures and the like. Preferably, these substances are imparting a pleasant odor.

The proportion of the active ingredient to the total oil phase is approximately from 15% to 100% by weight and preferably from 30% to 100% by weight and even more preferred from 40% to 100% by weight and even most preferred from 50% to 100% by weight relative to the total weight of the oil phase.

Thus, with the process described herein, it is possible to efficiently encapsulate a considerable amount of active ingredient(s).

Alternatively, and preferably, the active ingredient itself, for example a perfume oil can serve as the oil component in which the at least one polyisocyanate is dissolved.

The inventive eco-friendly core-shell microcapsules allow for the efficient encapsulation of a variety of active ingredients and thus allow for their use for the preparation of numerous products and the incorporation into a wide range of product formulations.

Moreover, the first stage of the process described herein, requires the provision of a first aqueous phase comprising at least one capsule formation aid, i.e., one or more capsule formation aids. For this purpose, the capsule formation aid is dissolved in the aqueous solvent (preferably pure water) to form the first aqueous phase.

The capsule formation aid(s) used within the scope of the present invention are for example surface-active agents (surfactants or emulsifiers) and/or colloidal protective agents.

Generally, a surface-active agent, or simply a surfactant, is a substance which lowers the surface tension between two different phases, and which is usually amphiphilic in nature having both, hydrophobic and hydrophilic functional groups. Consequently, these substances are able to arrange at the interface of an oil phase and an aqueous phase such as around the droplets of oil-in-water emulsions allowing for the stabilization of finely spread discrete oil droplets in the surrounding aqueous phase and thus avoiding an aggregation of said oil droplets. Therefore, these surface-active agents may act as emulsifiers, i.e., substance that stabilizes an emulsion by increasing its kinetic stability and reducing the interfacial tension between the two phases.

Therefore, in order to facilitate the emulsification, it can be useful to add such emulsifiers as capsule formation aids selected from the group of non-ionic, anionic, amphoteric, and/or cationic surfactants and mixtures thereof to the first aqueous phase.

Suitable non-ionic emulsifiers encompass for example:
products of the addition of 2 mol to 30 mol ethylene oxide and/or 0 mol to 5 mol propylene oxide onto linear C₈₋₂₂ fatty alcohols, onto C₁₂₋₂₂ fatty acids and onto alkyl phenols containing 8 to 15 carbon atoms in the alkyl group;
C_{12/18} fatty acid monoesters and diesters of addition products of 1 mol to 30 mol ethylene oxide onto glycerol;
glycerol mono- and diesters and sorbitan mono- and diesters of saturated and unsaturated fatty acids containing 6 to 22 carbon atoms and ethylene oxide addition products thereof;
addition products of 15 mol to 60 mol ethylene oxide onto castor oil and/or hydrogenated castor oil;
polyol esters and, in particular, polyglycerol esters such as, for example, polyglycerol polyricinoleate, polyglycerol poly-12-hydroxystearate or polyglycerol dimerate isostearate. Mixtures of compounds from several of these classes are also suitable;
addition products of 2 mol to 15 mol ethylene oxide onto castor oil and/or hydrogenated castor oil;
partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose);
mono-, di- and trialkyl phosphates and mono-, di- and/or tri-PEG-alkyl phosphates and salts thereof;
wool wax alcohols;
polysiloxane/polyalkyl polyether copolymers and corresponding derivatives;
mixed esters of pentaerythritol, fatty acids, citric acid and fatty alcohol and/or mixed esters of C₆₋₂₂ fatty acids, methyl glucose and polyols, preferably glycerol or polyglycerol,
polyalkylene glycols and
glycerol carbonate.

The addition products of ethylene oxide and/or propylene oxide onto fatty alcohols, fatty acids, alkylphenols, glycerol mono- and diesters and sorbitan mono- and diesters of fatty acids or onto castor oil are known and commercially available products. They are homologue mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations. In the following, preferred emulsifiers are described in more detail:

Partial glycerides: Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 mol to 30 mol and preferably of 5 mol to 10 mol ethylene oxide onto the partial glycerides mentioned are also suitable.

Sorbitan esters: Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 mol to 30 mol and preferably 5 mol to 10 mol ethylene oxide onto the sorbitan esters mentioned are also suitable.

Polyglycerol esters: Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 mol to 30 mol ethylene oxide.

Tetraalkyl ammonium salts: Cationically active surfactants comprise the hydrophobic high molecular group required for the surface activity in the cation by dissociation in aqueous solution. A group of important representatives of the cationic surfactants are the tetraalkyl ammonium salts of the general formula: (R¹R²R³R⁴N⁺) X⁻. Here R¹ stands for C₁-C₈ alk(en)yl, R², R³ and R⁴, independently of each other, for alk(en)yl radicals having 1 to 22 carbon atoms. X is a counter ion, preferably selected from the group of the halides, alkyl sulfates and alkyl carbonates. Cationic surfactants, in which the nitrogen group is substituted with two long acyl groups and two short alk(en)yl groups, are particularly preferred.

Esterquats: A further class of cationic surfactants particularly useful as co-surfactants for the present invention is represented by the so-called esterquats. Esterquats are generally understood to be quaternized fatty acid triethanolamine ester salts. These are known compounds which can be obtained by the relevant methods of preparative organic chemistry. Reference is made in this connection to international patent application WO 91/01295 A1, according to which triethanolamine is partly esterified with fatty acids in the presence of hypophosphorous acid, air is passed through the reaction mixture and the whole is then quaternized with dimethyl sulphate or ethylene oxide. In addition, German patent DE 4308794 C₁ describes a process for the production of solid esterquats in which the quaternization of triethanolamine esters is carried out in the presence of suitable dispersants, preferably fatty alcohols.

Typical examples of esterquats suitable for use in accordance with the invention are products of which the acyl component derives from monocarboxylic acids corresponding to formula RCOOH in which RCO is an acyl group containing 6 to 10 carbon atoms, and the amine component is triethanolamine (TEA). Examples of such monocarboxylic acids are caproic acid, caprylic acid, capric acid and technical mixtures thereof such as, for example, so-called head-fractionated fatty acid. Esterquats of which the acyl component derives from monocarboxylic acids containing 8 to 10 carbon atoms, are preferably used. Other esterquats are those of which the acyl component derives from dicarboxylic acids like malonic acid, succinic acid, maleic acid, fumaric acid, glutaric acid, sorbic acid, pimelic acid, azelaic acid, sebacic acid and/or dodecanedioic acid, but preferably adipic acid. Overall, esterquats of which the acyl component derives from mixtures of monocarboxylic acids containing 6 to 22 carbon atoms, and adipic acid are preferably used. The molar ratio of mono and dicarboxylic acids in the final esterquat may be in the range from 1:99 to 99:1 and is preferably in the range from 50:50 to 90:10 and more particularly in the range from 70:30 to 80:20. Besides the quaternized fatty acid triethanolamine ester salts, other suitable esterquats are quaternized ester salts of mono-/dicarboxylic acid mixtures with diethanolalkyamines or 1,2-dihydroxypropyl dialkylamines. The esterquats may be obtained both from fatty acids and from the corresponding triglycerides in admixture with the corresponding dicarboxylic acids. One such process, which is intended to be representative of the relevant prior art, is proposed in European patent EP 0750606 B1. To produce the quaternized esters, the mixtures of mono- and dicarboxylic acids and the triethanolamine-based on the available carboxyl functions—may be used in a molar ratio of 1.1:1 to 3:1. With the performance properties of the esterquats in mind, a ratio of 1.2:1 to 2.2:1 and preferably 1.5:1 to 1.9:1 has proved to be particularly advantageous. The preferred esterquats are technical mixtures of mono-, di- and triesters with an average degree of esterification of 1.5 to 1.9.

Further suitable surfactants are nonionic polymers such as sorbitan ester ethoxylate (Tween®) and/or polypropylene oxide/ethylene co-polymer, Tergitol™, Triton™ (Dow Chemicals), and alcohol ethoxylates.

The use of a combination of anionic and/or amphoteric surfactants with one or more nonionic surfactants is further advantageous. In a preferred embodiment according to the invention the composition further comprises emulsifiers that do not cause agglomeration in the required product formulations selected from the group consisting of:
Alkyl phosphate derivatives;
Glyceryl oleate citrate derivatives;
Glyceryl stearate citrate derivatives;
Stearic acid esters;
Sorbitan esters;
Ethoxylated sorbitan esters;
Ethoxylated mono-, di- and triglycerides;
Methyl glucose esters.

Alternatively, or additionally, colloidal protective agents (so-called protective colloids) can be dissolved in the first aqueous phase as capsule formation aid(s).

Preferably, the first aqueous phase comprises one or more colloidal protective agent(s) as the capsule formation aid(s).

Therefore, in a preferred variant of the present invention, one or more colloidal protective agent(s) are used as the at least one capsule formation aid.

In an alternative embodiment, both, at least one colloidal protective agent and at least one surfactant are used for the first aqueous phase.

Preferably, the one or more colloidal protective agents are selected from the group consisting of polymers such as for example, polyvinyl alcohol (hydrolyzation: 70% or more), for example Selvol™ (Sekisui Specialty Chemicals), polyvinylpyrrolidones, polyvinyl acetates, chemically modified biopolymers, preferably chemically modified starches, modified gum Arabic, modified cellulose or cellulose derivatives such as methylcellulose, hydroxy propyl methyl cellulose and hydroxyethyl cellulose, gelatin, casein, acrylate polymers or mixtures thereof. Also suitable are for examples starches (often derived from maize, quinoa, oats, waxy barley, or potato), gum Arabic, or cellulose which is chemically modified for example by octenyl succinic anhydride (OSA). Respective products are obtainable for example as Capsul® Starch or Hi-CAP® 100 (Ingredion Inc.).

Preferably, the colloidal protective agent(s) is/are, for example selected from the group consisting of known protective colloids such as polyvinyl alcohols, polyvinylpyrrolidones, hydroxyethyl cellulose, methylcellulose, gelatin, casein, polymethacrylic acid, and mixtures thereof.

It was found that polyvinyl alcohol is a suitable protective colloid for all types of biobased microcapsules according to the invention (see Examples 1 to 12). Moreover, polyvinyl alcohol is biodegradable under both aerobic and anaerobic conditions and therefore particularly suitable for the preparation of biodegradable and thus environmentally compatible microcapsules.

Furthermore, the protective colloid can be an anionic or amphiphilic polymer which is preferably selected from the group consisting of modified gum Arabic, octenylsuccinic anhydride modified starch (OSA) such as Capsul®, HiCap™ soy protein, sodium caseinate, gelatin, bovine serum albumin, sugar beet pectin, hydrolyzed soy protein, hydrolyzed sericin, Pseudocollagen, Biopolymer SA-N, Pentacare-NA PF, a mixture of gum Arabic and Revitalin, and mixtures thereof.

Preferably said protective colloid is a biopolymer (biomacromolecule), i.e., a material extracted from renewable sources or produced by living organisms (i.e., being of biological origin; bio-derived) such as polysaccharides, e.g., starches or celluloses. Biopolymers are characterized by molecular weight distributions ranging from 1 000 Daltons to 1 000 000 000 Daltons and can for example be carbohydrates (sugar based), proteins (amino acid-based) or a combination of both and can be linear or branched. Biopolymeric derivatives (also called biobased polymers) are compounds derived from such biomacromolecules by means of chemical derivatization. Usually, these substances are also biodegradable. Preferably, one or more protective colloid(s) is/are selected from the group consisting of modified gum Arabic, octenylsuccinic anhydride modified starch (OSA) such as Capsul®, HiCap™ or (modified/derivatized) soy proteins.

Considering the objective of the present invention, the at least one capsule formation aid used within the scope of the present invention is preferably a colloidal protective agent, wherein biopolymeric compounds are especially preferred as the capsule formation aid(s), especially in view of an eco-friendly approach.

A comparison between Example 1 and Example 5 indicates, that such biobased protective colloids can suitably be used for the preparation of biobased microcapsules according to the invention showing high stabilities and simultaneously high release properties.

Therefore, in a preferred variant, the invention relates to a process for the preparation of core-shell microcapsules or a slurry comprising core-shell microcapsules according to the invention, wherein the at least one formation aid is a biopolymer or a biopolymeric derivative, and preferably a biopolymeric derivative.

However, in a further preferred variant, the at least one capsule formation aid is polyvinyl alcohol, allowing for the formation of highly stable and excellently performing microcapsules as indicated above.

Based on these substances specified herein as colloidal protective agents, it is possible to prepare environmentally friendly and simultaneously efficient microcapsules having ideally balanced capsule properties.

The microcapsules according to the present invention can be prepared by using one or more colloidal protective agents and/or one or more surface active agents as specified herein. Preferably one or more colloidal protective agents are used. Even more preferred these colloidal protective agents are biodegradable and/or biobased or bioderived.

Typically, the capsule formation aid(s) is/are comprised in the first aqueous phase in amounts of from 0.1% to 5% by weight and preferably from 0.5% to 5% by weight.

If the capsule formation aid(s), for example the protective colloid(s), is/are contained within these amounts a high degree of stabilization of the oil droplets in the continuous aqueous phase can be achieved. Furthermore, settling is avoided and clumping effects are reduced, thus keeping the oil particles finely dispersed within the surrounding aqueous phase thereby guaranteeing a homogeneous encapsulation and homogeneous size distribution of the resulting microcapsules.

In addition, continuous agitation during the preparation process according to the invention helps avoiding the agglomeration and thus clumping of the particles and/or the resulting microcapsules according to the invention as well as the settling of the microcapsules.

Moreover, the pH-value of the first aqueous phase is preferably 7 or higher. Preferably the pH is approximately 8 or higher. In order to achieve these pH-values corresponding acid or basic substances can be added to the first aqueous phase or alternatively to the preliminary oil-in-water emulsion. Alternatively, the pH-adjustment (if required) is performed in the second aqueous phase or after the addition of said second aqueous phase.

Furthermore, the inventive process requires the provision of a second aqueous phase comprising at least one saccharide and/or aminosaccharide as building blocks each independently having less than 20 monomeric units (or repeat units), i.e., equal to 19 or less monomeric units, and preferably 15 or less monomeric units, and even more preferred 10 or less monomeric units, optionally wherein the second aqueous phase further comprises one or more additional formation aid(s). For this purpose, the at least one saccharide and/or aminosaccharide each independently having less than 20 monomeric units, preferably 15 or less monomeric units, even more preferred 10 or less monomeric units and optionally the capsule formation aid(s) are dissolved in an aqueous solvent (preferably pure water).

However, in an alternative variant of the inventive process it is also possible to directly add the (amino)saccharide(s) into the first aqueous phase and omit the step of providing a second aqueous phase and the preparation of an intermediate oil-in-water emulsion. Thereby, the isocyanate-containing oil phase and the (amino)saccharide- and formation aid-containing aqueous phase are blended to obtain an oil-in-water emulsion followed by curing. If required, the pH-value is adjusted by adding alkaline solutions.

In the following the (amino)saccharide component of the inventive core-shell microcapsules as well as the corresponding components used within the inventive process are described:

Typically, such monomeric, dimeric, oligomeric and small, i.e., short-chained polymeric (amino)saccharides have molecular weights of 3 500 Da or less and preferably less than 3 000 Da. Therefore, within the scope of the present invention preferably aminosaccharides and/or saccharides having a molecular weight of 3 500 Da or less and preferably 3 000 Da or less are used as building blocks for the preparation of highly stable and efficient biobased core-shell microcapsules.

The present invention uses specific ratios of mono, di- and oligosaccharides as well as smaller, i.e., short-chained polysaccharides reacted with polyisocyanates to form structural segment units forming the shell wall in a core-shell microcapsule. The saccharides suitable within the scope of the present invention are carbohydrates or derivatives thereof, i.e., derivatives of biomolecules consisting of carbon, hydrogen and oxygen atoms. These saccharides generally can be divided into four chemical groups based on the number of monomeric units, i.e., monomeric saccharide building blocks: monosaccharides (one monomeric unit), disaccharides (two monomeric units), oligosaccharides (three to 10 monomeric units), and polysaccharides (more than 10 monomeric units). Thereby each monomeric unit, i.e., saccharide building block has two or more hydroxyl groups (—OH). Oligosaccharides are defined as short polymers of monosaccharide residues linked by glycoside bonds having a degree of polymerization (DP) between three and ten units (IUPAC definition). They may be linear or branched and normally contain hexoses or pentoses individually or in mixtures. Other monosaccharides may be present including uronic acids, sialic acids and anhydro sugars. Dimeric, oligomeric and polymeric saccharides can be composed of one type of monomeric unit or of more than one type of monomeric units, i.e., two or more structurally different monomeric units.

Such carbohydrates which are most preferably used within the scope of the invention are saccharides (colloquial: sugars) with less than 20 monomeric units, i.e., less than 20 linked monosaccharide resides, preferably 15 or less monomeric units and even more preferred 10 or less monomeric units, and are for example monosaccharides (glucose, fructose, galactose), disaccharides (sucrose, lactose, maltose), linear or branched oligosaccharides (raffinose, stachyose) and/or linear or branched short-chained polysaccharides as defined herein.

The term saccharide as used herein includes not only saccharides obtained from natural sources but also synthetically produced saccharides and derivatives thereof of equivalent structure to natural saccharides.

However, preferably, the crosslinked saccharide building blocks used within the scope of the present application are mono-, di- and/or oligosaccharides and/or short-chained polysaccharides as defined herein. Preferably these saccharides are from natural sources.

The smaller size of the molecules allows for a facilitated diffusion towards the oil core comprising the isocyanate linker which is surrounded by the capsule formation aid(s) and thus allows for enhanced interfacial polymerization with a denser crosslinking resulting in more stable capsules with simultaneously enhanced performances which allow for the efficient encapsulation of the active ingredient(s) as core materials. Simultaneously the biobased capsule building blocks allow for an improved biodegradability of the capsule wall material compared to fully synthetic state-of-the-art microcapsules commercially available. The stability of the capsules as well as their performance is improved compared to microcapsules being based on larger building blocks such as starch or chitosan.

Generally, the amine or alcohol building-block permeates from the aqueous phase into the organic phase for wall formation. Thereby the permeation rate is primarily controlled by the solubility and diffusion rate of said building blocks, with the molecular size of the building blocks considerably influencing the diffusion rate and thus the permeation rate. For larger building blocks such as commercially available starch or chitosan the diffusion rate and the permeation rate are considerably reduced resulting in a reduced crosslinking density and less efficient encapsulation as well as reduced capsule stabilities. Consequently, also the release performance of microcapsules being based on large building blocks such as starch and/or chitosan is significantly reduced.

In the corresponding aminosaccharides one or more of the hydroxyl groups of the saccharide structure are substituted by an amine group (—NH₂ or —NH—, and preferably —NH₂). Such amino functional mono-, di-, oligo- and polysaccharides are for example: glucosamine, mannosamine, galactosamine, poly- and oligoglucosamines, such as oligochitosans, also known as chitoses, chitooligosaccharides or chitosan oligosaccharides (having less than 20 monomeric units) and chitosan (20 or more monomeric units), and N-acetyl substituted aminosaccharides. Preferably, the aminosaccharides used within the scope of the present invention have less than 20 monomeric units, i.e., equal to 19 or less monomeric units and preferably 15 or less monomeric units, even more preferred 10 or less monomeric units. Therefore, the aminosaccharides used within the context of the present invention are preferably monomeric aminosaccharides (one monomeric unit), dimeric aminosaccharides (two monomeric units), linear or branched oligoaminosaccharides (oligomeric aminosaccharides; three to 10 monomeric units) and/or short-chained polyaminosaccharides (polymeric aminosaccharides; more than 10 monomeric units) having chain-lengths of less than 20 monomeric units and preferably 15 or less monomeric units and being branched or linear. Dimeric, oligomeric and polymeric aminosaccharides can be composed of one type of monomeric unit or of more than one type of monomeric units, i.e., two or more structurally different monomeric units.

Correspondingly, in a preferred variant, the crosslinked aminosaccharide building blocks used within the scope of the present application are monomeric, dimeric and/or oligomeric and/or short-chained polymeric aminosaccharides as defined herein, which are preferably from natural sources.

The term aminosaccharide as used herein includes not only aminosaccharides obtained from natural sources but also synthetically produced aminosaccharides and derivatives thereof of equivalent structure to natural aminosaccharides. However, preferably aminosaccharides from natural sources are used within the scope of the present invention.

Moreover, saccharides comprising or consisting of a mixture of amine-free and amine-containing monomeric units can be suitably used within the scope of the present invention and are also referred to as “aminosaccharides”. Preferably, in an aminosaccharide, however, all of the monomer units comprise amine groups.

In a further preferred variant, the saccharide and/or aminosaccharide building blocks each independently having less than 20 monomeric units are either linear or branched. For forming the capsule wall, a combination of two or more linear and/or branched building blocks can be used, i.e., a mixture of one or more linear saccharides, one or more linear aminosaccharides, one or more branched saccharides and/or one or more branched aminosaccharides as well as mixtures of linear and branched saccharides and/or aminosaccharides.

According to a preferred variant of the present invention the capsule shell is formed of a polymeric material being a reaction product of at least one polyisocyanate having at least two isocyanate groups, i.e., a single isocyanate compound or a mixture of two or more different isocyanate compounds each having two or more isocyanate functionalities, and a at least one saccharide as defined herein each independently having less than 20 monomeric units and preferably 15 or less monomeric units and even more preferred 10 or less monomeric units.

Alternatively, the capsule shell is formed of a polymeric material being a reaction product of at least one polyisocyanate having at least two isocyanate groups, i.e., a single isocyanate compound or a mixture of two or more different isocyanate compounds each having two or more isocyanate functionalities, and one or more aminosaccharides each independently having less than 20 monomeric units and preferably 15 or less monomeric units and even more preferred 10 or less monomeric units as defined herein.

However, in a further preferred variant the capsule shell is formed of a polymeric material being a reaction product of at least one polyisocyanate having at least two isocyanate groups, i.e., a single isocyanate compound or a mixture of two or more different isocyanate compounds each having two or more isocyanate functionalities, and at least one saccharide and at least one aminosaccharides as defined herein, i.e., a combination or mixture of at least one (i.e., one or more) saccharide and at least one (i.e., one or more) aminosaccharide building blocks, the saccharide(s) and aminosaccharide(s) each independently having less than 20 monomeric units and preferably 15 or less monomeric units and even more preferred 10 or less monomeric units.

Based on these polymeric materials highly efficient microcapsules (i.e., microcapsules being stable towards mechanical impact or heat or in product formulations and which are able to efficiently encapsulate the active ingredient until a targeted release is initiated but which simultaneously show a high release performance) can be prepared which at the same time show increased biodegradability properties.

Thereby, the (amino)saccharide units serve as sites for biological attack and thus biological degradation, thus reducing the environmental impact of the capsule material due to their enhanced degradability over state-of-the-art microcapsules, therefore allowing for the preparation of highly efficient and simultaneously environmentally more compatible and thus environmentally more friendly microcapsule alternatives.

The capsule shell of the inventive core-shell microcapsules is formed by interfacial polymerization between the functional groups of the polyisocyanates and the functional hydroxy and/or amine groups of the (amino)saccharide units resulting in polyurethane and/or polyurea-based crosslinkages and thus polymeric structures around the core comprising the active ingredient as specified above. Thereby, the isocyanate units serve as “crosslinks” or “hard” segments whereas the (amino)saccharide units serve as the hydrophilic “soft” segments that form the major portion of the shell material.

According to the invention the term “saccharide” refers to both, saccharide structures comprising hydroxy functionalities and/or saccharides comprising one or more amine functionalities, i.e., aminosaccharides for the sake of simplicity.

The polyaddition reaction of the at least one polyisocyanate with the hydroxyl groups of the at least one (amino)saccharide leads to the formation of so-called urethane bridges (—NH—CO—C—) by addition of the hydroxyl groups of the (amino)saccharide(s) (—OH) to the carbon atom of the carbon-nitrogen bond of the isocyanate groups (—N═C═O). Alternatively, or simultaneously, the formation of the polyurea linkage takes place in a similar way to the formation of the polyurethane linkage by polyaddition of the amine group of the aminosaccharide(s) (—NH₂, —NH—) to the isocyanate functionality of the polyisocyanate(s). Alternatively, in the case of the corresponding thiosaccharides polythiourethane linkages are formed.

Thus, a tightly crosslinked capsule shell can be formed as polymeric material from crystalline, polar segments and/or tightly crosslinked segments with reduced polyisocyanate content which prevents the diffusion or permeation of the hydrophobic active ingredient (or mixture of active ingredients) enclosed in the capsule as core material. In the case of fragrances and perfumes, for example, this leads to an effective encapsulation of the sensorially perceivable active ingredient (fragrance mixture or single fragrance substance), which is effectively released by e.g., mechanical activation or by external environmental changes such as changes in the pH-value or temperature or through biological attack. This requires additionally that the capsules show sufficient stability so that a high performance can be achieved. Based on the inventive biobased core-shell microcapsules it is possible to provide for highly stable capsules which allow for the efficient encapsulation of the active ingredient(s) without losses due to an evaporation of the active(s) and/or its interaction with other components of the product formulation and/or due to premature breakage during application (e.g., in the washing machine etc.). Simultaneously the inventive biobased capsules show a high performance and thus allow for the efficient and targeted release of the active(s). In addition, they show increased biodegradability, without negatively influencing the stability nor the performance.

Additionally, it was surprisingly found that it is possible to prepare highly stable bio-derived and eco-friendly microcapsules with considerably reduced shell materials (see Examples 11 and 12). These capsules show high mechanical, thermal and chemical stability despite having a reduced shell material and low isocyanate content due to the efficient crosslinking of the (amino)saccharide segments as defined herein.

The higher the number of crosslinking functional groups is, the greater the spatial crosslinking and the more stable the resulting capsule shell or capsule wall of the final microcapsule. In addition to the number of functional groups, i.e., the number of branches, the chain length of the individual building blocks has a significant influence on the mechanical properties, i.e., the stability of the capsules. However, too high crosslinking will increase the capsule stability but would simultaneously negatively influence the biodegradability and performance, i.e., the release behavior of the capsule material and the capsules as such. However, based on the process described herein and the (amino)saccharides specified herein according to the first aspect it is possible to achieve microcapsules having an enhanced or even ideal balance of these properties, and thus these building blocks allow for the preparation of highly stable microcapsules with excellent performances and increased biodegradability which are suitable for the stable incorporation in various consumer product formulations.

The inventive microcapsules according to the first aspect exhibit a high mechanical stability against mechanical influences, such as those prevailing in a tumble dryer or heat (thermal stability), but at the same time allow for an efficient targeted, signal-induced release of the active ingredient. In addition, sensory experiments have shown that, in the case of fragrances as active ingredients, high fragrance intensity could be perceived, which suggests that the fragrances were efficiently enclosed within the inventive core-shell microcapsules indicating the efficient reduction of losses due to diffusion of the active ingredient out of the microcapsule even after aging within the consumer product formulation. The biobased core-shell microcapsule, wherein the shell comprises at least one saccharide and/or at least one aminosaccharide each independently having less than 20 monomeric units as defined herein, are highly stable and allow for an efficient encapsulation of active ingredients and high performances despite being based on biobased components. Simultaneously they show an increased biodegradability compared to commercially available state-of-the-art microcapsules.

It was additionally found that a combination of different saccharides and/or aminosaccharides can efficiently be used to regulate the crosslink density in the shell wall and thus the resulting capsule properties.

Therefore, the present invention primarily focuses on (amino)saccharide-based core-shell microcapsules. And more specifically, on (amino)saccharide-based core-shell microcapsules, wherein the shell comprises or consists of a polymeric material being a reaction product of at least one polyisocyanate having at least two isocyanate groups and at least one (amino)saccharide, i.e., at least one saccharide and/or at least one aminosaccharide, each independently having less than 20 monomeric units and wherein the core comprises or consists of at least one active ingredient.

Saccharide-based within the scope of the present invention means that at least one saccharide and/or at least one aminosaccharide as specified herein is used as building block for the formation of the capsule shell.

Biobased or bio-derived within the scope of the present invention means that the materials used within the scope of the present application are preferably derived from replenishable, natural resources and more specifically, from renewable and biogenic sources like agricultural crops, agricultural residues or wood. For example, the (amino)saccharides described herein refer to natural biocompounds (i.e., chemical compounds of biological origin), and more specifically a class of natural molecules. It was surprisingly found that these bio-derived materials can be suitably used as the main building blocks for the preparation of stable and efficient biobased/bio-derived or (amino)saccharide-based core-shell microcapsules and thus as biobased and more environmentally friendly alternatives to fossil-based microcapsules.

Thereby, it has been found that specific monomeric, dimeric and oligomeric (amino)saccharides as well as short-chained polymeric (amino)saccharides as specified herein such as low molecular weight maltodextrins with less than 20 monomeric units (or repeat units), and preferably 15 or less monomeric units and even more preferred 10 or less monomeric units are particularly advantageous in the shell formation of said bio-derived core-shell microcapsules. These low molecular weight (amino)saccharides diffuse more readily into the reaction interface compared to large molecular weight poly(amino)saccharides. The diffusion is less hindered, and the wall formation facilitated allowing for the formation of a higher degree of crosslinking resulting in more stable and simultaneously better performing microcapsules. The capsules efficiently encapsulate the active ingredient without losses due to diffusion of the active ingredient(s) and can stably be incorporated in various formulations, in particular consumer products such as detergents, fabric softeners and the like. Moreover, these biobased microparticles show a high performance, i.e., excellent fragrance release properties in the case of fragrance or odorant capsules and increased biodegradability.

Aminosaccharides occur widely in nature, having been found in a wide range of compounds such as structural polysaccharides, mucopolysaccharides, bacterial capsular polysaccharides, teichoic acids, lipopolysaccharides, glycolipids, mucoproteins, nucleotides, and the like.

Chitosan, for example, is a generally nontoxic, biodegradable, and biocompatible polysaccharide, i.e., a biopolymer, based on (1-4)-linked D-glucosamine monomeric units and N-acetyl-D-glucosamine monomeric units having a molecular weight in the range of 30 000 Da to 50 000 Da or more, whereby the polymeric molecule comprises 20 or more monomeric units, i.e., glucosamine-based monomeric units (D-glucosamine or N-acetyl-D-glucosamine monomeric units; each being considered a monomeric unit individually) and usually even more than 100 monomeric units. However, chitosan has only a poor solubility in water or in organic solvents and is only soluble to a few percent (1-3%) in organic acidic solutions such as acetic acid, propionic acid, citric acid, and formic acid. In addition, acidified chitosan solution has a very poor reactivity with polyisocyanates in an interfacial reaction. Therefore, chitosan as such is not suitable for the preparation of core-shell microcapsules. In addition, due to the large structure of the molecule, the diffusion towards the isocyanate reactant is hindered resulting in less dense cross-linkages and thus less efficient encapsulations with reduced stabilities.

However, it was now surprisingly discovered that specific low molecular weight chitosan oligomers having less than 20 monomeric units and preferably 15 or less monomeric units and even more preferred 10 or less monomeric units, which are also known as oligochitoses (or chito-oligosaccharides) and which are the degraded products of chitosan or chitin prepared by enzymatic or chemical hydrolysis of chitosan (Carbosynth-Biosynth® LTD, England) and derivatives thereof, overcome the poor solubility in both acidic and alkaline solvents and allow for solubilities at higher levels exceeding 10%.

Per definition, according to the invention, chitosan derivatives having a degree of polymerization (DPs) of less than 20 and an average molecular weight of less than approximately 3 900 Da are referred to as chitosan oligomers, chitooligomers, oligochitoses or chito-oligosaccharides (COS). These terms are used synonymously. The definition of what constitutes a chitosan oligomer may deviate from the classic definition of oligomeric structures as defined above having less than 10 monomeric units. However, according to the present invention chitosan oligomers are defined as having less than 20 monomeric units and are therefore also referred to as “oligoaminosaccharides” or “oligomeric aminosaccharides” or alternatively, “short-chained polyaminosaccharides”.

The uniqueness of the low molecular weight chitosan oligomers (MW<3 900 g/mol and preferably <3 000 g/mol; also known as chito-oligosaccharides or oligochitoses) allows for a higher solubility in both acidic and basic medias compared to common chitosan having a molecular weight of 30 000 g/mol to 5 000 000 g/mol or higher. In addition, the chito-oligosaccharides are readily soluble in water due to their shorter chain lengths and thus in dependence of the degree of polymerization (DP). The chitosan oligomer having a molecular weight of less than 3 000 g/mol is based on 10 to 15 monomeric glucosamine units (D-glucosamine or N-acetyl-D-glucosamine monomeric units). This permits an increased reaction with the isocyanate components and a direct incorporation as a segment into the shell of the core-shell microcapsules as opposed to a coating as this aminosaccharide is able to efficiently polymerize with the crosslinkers, i.e., polyisocyanates, to form structural units in the shell wall. Moreover, the smaller size of the building blocks allows for a denser crosslinking. Therefore, these compounds are suitable for the preparation of saccharide-based or biobased core-shell microcapsules. Consequently, chitosan in its oligosaccharide form is preferably used within the scope of the present invention. Thereby, for example formulations with different mixtures of glucosamine and oligochitoses can be used to regulate the crosslink density in the shell wall and its properties.

Preferably, the chito-oligosaccharides and its derivatives suitably used within the context of the present invention have a molecular weight of less than 3 000 g/mol or 3 000 Da.

In a further preferred variant, the chito-oligosaccharides comprise less than 20 monomeric units and preferably 15 or less monomeric units and even more preferred 10 or less monomeric units.

In addition, the smaller size of the molecule allows for a facilitated diffusion towards the oil core comprising the isocyanate linker which is surrounded by the capsule formation aid allowing for the efficient formation of crosslinks between the isocyanate component and the biobased crosslinker, i.e., the saccharide and/or aminosaccharide each independently having less than 20 monomeric units, preferably 15 or less monomeric units, even more preferred 10 or less monomeric units. Thereby a biobased or (amino)saccharide-based polymeric material is formed as reaction product forming the capsule shells.

As indicated above, biobased reagents are preferred in view of health and environmental aspects. Therefore, the saccharide(s) and/or aminosaccharide(s) and/or polyisocyanate(s) used for the preparation of the inventive core-shell microcapsules are preferably bio-derived. Preferably, also the capsule formation aids are bio-derived or at least biodegradable.

Furthermore, small alkaline or neutral chitosan oligomers, i.e., chito-oligosaccharides, as described above allow for an improved interfacial reaction with isocyanate crosslinkers dissolved in the oil phase compared to the long-chained chitosan. It was further found that polyisocyanates react more favorable under basic, i.e., alkaline conditions than under acidic conditions with the amino groups of aminosaccharides such as glucosamine and the short-chained chitosan oligomers. Therefore, preferably the pH-value is kept above 8 during the preparation process described herein. Thus, preferably the pH of the first and/or second aqueous phase is in the alkaline range and preferably above 8.

Therefore, in a preferred variant of the present invention, the (amino)saccharides preferably used within the scope of the invention are (amino)saccharides each independently having less than 20 monomeric units, preferably 15 or less monomeric units and even more preferred 10 or less monomeric units. According to the present invention, at least one such saccharide and/or at least one aminosaccharide is reacted with the polyisocyanate(s).

In another preferred embodiment preferably a mixture of at least one saccharide and at least one aminosaccharide as defined herein is used.

Preferably, the inventive core-shell microcapsule comprises or consists of a polymeric material being a reaction product of at least one polyisocyanate having at least two isocyanate groups and at least one saccharide and/or at least one aminosaccharide each independently having less than 20 monomeric units selected from the following group: glucosamine, maltodextrin and/or chito-oligosaccharides, wherein the core comprises or consists of at least one active ingredient.

These (amino)building blocks alone or in mixture allow for the formation of highly stable, but simultaneously excellently performing microcapsules which are stable in consumer product formulations for a long time (at least four weeks) and which show increased biodegradability. For chito-oligosaccharide based capsules see e.g., Examples 2 and 3 and for maltodextrin-based capsules see e.g., Examples 6 to 8 and 10.

However, in an even more preferred embodiment, the at least one (amino)saccharide-component having less than 20 monomeric units is monomeric (i.e., a monosaccharide or monomeric aminosaccharide) and even more preferred the (amino)saccharide-based building block is glucosamine (see Examples 1 or 5).

Glucosamine can either be used as the only (amino)saccharide building block or can also be suitably combined with other (amino)saccharide building blocks as defined herein such as maltodextrin and/or chito-oligosaccharides. Glucosamine is a monomeric aminosaccharide compound and one of the most common monosaccharides which is even used as dietary supplement. Core-shell microcapsules prepared based on glucosamine show excellent stabilities, also within product formulation, high release performances and improved biodegradability properties.

Within the scope of the present invention, generally, a small molecule such as glucosamine or glucose can be freely mixed with larger dimeric, oligomeric or short-chained (amino)saccharides as defined herein given the similarities in their structures.

Therefore, in preferred embodiment core-shell microcapsules are disclosed, wherein the shell comprises or consists of a polymeric material being a reaction product of at least one polyisocyanate having at least two isocyanate groups and at least one saccharide and/or at least one aminosaccharide each independently having less than 20 monomeric units, wherein the at least one saccharide and/or at least one aminosaccharide is glucosamine and wherein the core comprises or consists of at least one active ingredient.

The reaction of the polyisocyanates with the available amino and/or hydroxyl groups of the saccharide(s) and/or aminosaccharide(s) having less than 20 monomeric units, preferably 15 or less monomeric units and even more preferred 10 or less monomeric units, by forming urea and/or urethane-based crosslinks is exemplarily shown below for an aminosaccharide building block:

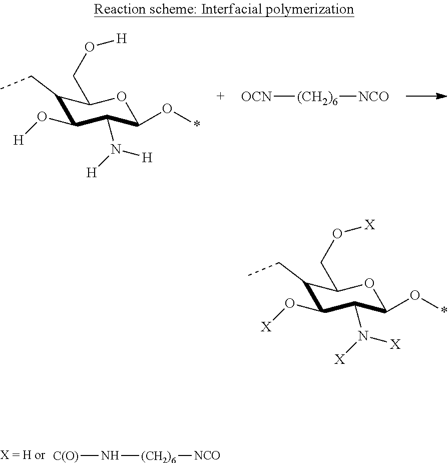

Consequently, based on the reaction of the polyisocyanates as defined herein and the (amino)saccharides as defined herein, it is possible to obtain core-shell microcapsules, wherein the shell material comprises or consists of a biobased polymeric material being a reaction product of at least one polyisocyanate, preferably at least one aliphatic polyisocyanate, having at least two isocyanate groups, i.e., a polyisocyanate having more than one isocyanate groups, and preferably a mixture of at least two polyisocyanates, preferably wherein at least one of the polyisocyanates in said mixture is aliphatic (or wherein all of the polyisocyanates in said mixture are aliphatic in nature), and at least one saccharide and/or at least one aminosaccharide each independently having less than 20 monomeric units, preferably 15 or less monomeric units and even more preferred 10 or less monomeric units, i.e., comprising or consisting of less than 20, 15 or less or 10 or less monomeric units, respectively, and wherein the core comprises or consists of at least one active ingredient, such as a fragrance compound.

Thereby, preferably, the at least one polyisocyanate is aliphatic. Even more preferred, a mixture of two or more polyisocyanates is used, wherein preferably at least one or all of the polyisocyanates is/are aliphatic. Alternatively, preferably at least one or all of the polyisocyanates of the mixture of polyisocyanates is/are aromatic. In a further alternative, a mixture of two or more polyisocyanates is used, wherein at least one of the polyisocyanates in aliphatic and at least one polyisocyanate is aromatic. Preferably, if mixtures of two or more polyisocyanates are used, it is preferred that said mixture comprises or consist of more than 80 mol-% of aliphatic components/polyisocyanates. In the latter case, preferably the molar or weight ratio of the aliphatic polyisocyanate(s) to the aromatic polyisocyanate(s) is ranging from 85 to 15, and even more preferred from 90 to 10 to 99:1.

The isocyanate component is preferably selected independently from the (amino)saccharide component. Thereby, the aliphatic/aromatic isocyanates form a regional stratification in the shell independently of the amine/alcohol component due to the large reactivity differences between aromatic and aliphatic isocyanates.

Therefore, in a further aspect the present invention relates to a core-shell microcapsule, wherein the shell comprises or consists of a polymeric material being a reaction product of at least one polyisocyanate having at least two isocyanate groups and at least one saccharide and/or at least one aminosaccharide each independently having less than 20 monomeric units and wherein the core comprises or consists of at least one active ingredient.

Thereby a combination of one or more saccharide(s) and/or aminosaccharide(s) is particularly preferred allowing for the adjustment of the crosslink-density and thus of the resulting capsule properties. This results in varying lengths of the linkages between the building blocks influencing the crosslink density and allowing for different functionalities of the capsules due to the different functional groups of the building blocks. Moreover, different degrees of polymerization and crosslinking are achieved providing for more flexible capsule wall compositions and positively influencing the overall capsule properties.

The polymerization involves the reaction of active hydrogens of the corresponding (amino)saccharide(s) for crosslinking: In order to achieve an efficient encapsulation based on the polymerization of the building blocks it is therefore further necessary, that the at least one saccharide and/or at least one aminosaccharide has at least two functional groups independently selected from the group consisting of: primary and secondary hydroxyl groups (—OH) as well as primary amine (—NH₂) and secondary amine groups (—NH—) allowing for the formation of polyurethane and/or polyurea-based linkages.

A primary hydroxyl group is a hydroxy group (—OH) which is bonded to a primary carbon atom, i.e., a carbon atom which has one carbon atom directly attached to it, while a secondary hydroxy group is bonded to a secondary carbon atom, i.e., a carbon atom which has two carbon atoms directly attached to it.

On the other hand, primary amines can be derived from ammonia by replacing one of three hydrogen atoms with a non-hydrogen group, i.e., the nitrogen atom is bound to a non-hydrogen atom, for example a carbon-containing group, so that two hydrogen atoms remain. When referring to secondary amines two of the three hydrogen atoms are replaced by a non-hydrogen group, i.e., in these secondary substituted amines, the nitrogen is bonded with two non-hydrogen atoms leaving only one hydrogen atom bonded to the nitrogen.

Preferably, the at least one saccharide has at least two hydroxyl groups while the at least one aminosaccharide has at least two functional groups selected from the list specified above, wherein at least one of these functional groups is an amine group, for the formation of polyurethane and/or polyurea-based linkages as these groups result in enhanced stabilities and denser crosslinked capsule shells while simultaneously allowing for an enhanced capsule release performance, i.e., release behavior.

In a further preferred variant the present invention relates to core-shell microcapsule according to the invention, wherein the at least one saccharide and/or at least one aminosaccharide having less than 20 monomeric units is selected from the group consisting of: monosaccharides such as glucose, galactose, fructose, xylose, mannose, arabinose, erythrose, threose, ribose, arabinose, lyxose, allose, altrose, talose, fucose, rhamnose, monomeric aminosaccharides such as glucosamine, galactosamine, N-acetylglucosamine, disaccharides such as sucrose, lactose, maltose, isomaltulose, trehalose, lactulose, cellobiose, chitobiose, isomaltose, isomaltulose, maltulose, dimeric aminosaccharides, linear and/or branched oligosaccharides such as malto-oligosaccharides such as maltodextrins, raffinose, stachyose, fructo-oligosaccharides, melicitose, umbelliferose, cyclodextrins, oligoaminosaccharides (oligomeric aminosaccharides) such as chitooligosaccharides, and the like or mixtures thereof.

Alternatively, or in combination with the (amino)saccharides specified above thiosaccharides can be suitably used having less than 20 monomeric units and preferably 15 or less monomeric units and even more preferred 10 or less monomeric units, having at least two functional groups independently selected from the group consisting of: primary and secondary hydroxyl groups (—OH) and thiol groups (—SH). Preferably, these thiosaccharides have one or more functional thiol groups (—SH) allowing for the formation of polythiourethane structures/linkages. Preferably at least one thiol group and at least one hydroxyl group are present allowing for the formation of both, polythiourethane and polyurethane-based structures.

A combination of at least one thiosaccharide with at least one saccharide and/or aminosaccharide results in a polymeric structure based on polythiourethane and polyurethane and/or polyurea-linkages between the building blocks.

In another preferred variant of the present invention, therefore, a core-shell microcapsule is described, wherein the shell comprises or consists of a polymeric material being a reaction product of at least one polyisocyanate having at least two isocyanate groups and preferably a mixtures of two or more polyisocyanates and at least one saccharide and/or at least one aminosaccharide and/or at least one thiosaccharide each independently having less than 20 monomeric units and wherein the core comprises or consists of at least one active ingredient.

In a further alternative also sugar alcohols containing one hydroxyl group (—OH) attached to each carbon atom such as sorbitol or mannitol can be used as building blocks for the reaction with the isocyanate components.

The proportion of the at least one saccharide and/or at least one aminosaccharide and/or at least one thiosaccharide, i.e., the (amino)saccharide and/or thiosaccharide component to the total oil phase is at least 0.2% by weight and preferably at least 0.5% by weight, or particularly preferably at least 1% by weight, of the total weight of the oil phase. The upper limit is ranging from 5% to 10% by weight. However excess (amino)saccharide and/or thiosaccharide building blocks, i.e., unreacted building blocks in the aqueous phase are generally desired to ensure that all polyisocyanate molecules have reacted.

The proportion of the saccharide component(s) (saccharide, aminosaccharide, thiosaccharide) to the total second aqueous phase is from 1% to 30% by weight and preferably from 5% to 20% by weight. If the proportion of the saccharide component(s) is within said ranges, ideal reaction conditions can be achieved for an efficient encapsulation.

As indicated above, the second aqueous phase optionally comprises one or more additional formation aids such as colloidal protective agents and/or surface-active agents as specified above, which might be added in amounts of approximately 0.1% to 5% by weight and preferably 0.2% to 2% by weight by weight based on the second aqueous phase.

The second aqueous solution may contain for example, at least one surface active agent, wherein said agent is preferably a non-ionic and/or cationic and/or anionic polymer, as indicated above.

Generally, the capsule formation aid(s) added to the second aqueous phase can be the same or different form the capsule formation aid(s) used in the first aqueous phase.

Preferably, the one or more capsule formation aid(s) used in the first and/or second aqueous phase of the process for preparing biobased core-shell microcapsules or a slurry comprising said core-shell microcapsules according to the invention is thus a surface-active agent and/or a colloidal protective agent, and preferably a colloidal protective agent as specified above. Moreover, preferably the formation aid(s) used is/are based on biopolymers.

Therefore, microcapsules according to the invention may thus be produced using at least one aqueous phase containing capsule formation aid(s). The amount of the additive(s) being, for example, in the range 0.1% to 5% by weight based on the respective phase.

Alternatively, these substances could be added to the final microcapsule slurry in order to increase the stability of finely dispersed microcapsules within the aqueous phase.

Suitable polymers which might be alternatively or additionally added to the second aqueous phase or to the final capsule slurry to improve the spreadability of the composition upon the skin or hair, or improve the water and/or sweat and/or rub-off resistancy of the formula and to improve the protection factor of the composition are for example: VP/Eicosene copolymers sold under the trade name Antaron™ V-220 by International Specialty Products, VP/Hexadecene copolymer sold under the trade names Antaron™ V-216 and Antaron™ V-516 by International Specialty Products, Tricontanyl PVP sold under the trade name Antaron™ WP-660 by International Specialty Products, Isohexadecane and Ethylene/Propylene/Styrene copolymer and Butylene/Styrene copolymer sold under the trade names Versagel® MC and MD by Penreco, Hydrogenated polyisobutene and Ethylene/Propylene/Styrene copolymer and Butylene/Styrene copolymer sold under the trade mane Versagel® ME by Penreco, Acrylates/Octylacrylamide Coploymers sold under the trade name Dermacryl® 79, Dermacryl® AQF and Dermacryl® LT by AkzoNobel, Polyurethanes such as PPG-17/IPDI/DMPA copolymer sold under the trade name Avalure™ UR 450 & 525 sold by Lubrizol, Polyurethanes-2 and -4 sold under the trade names Avalure™ UR-405, -410, -425, -430 and -445, -525 sold by Lubrizol, Polyurethane 5 and Butyl Acetate and isopropyl alcohol sold under the trade name Avalure™ UR-510 and -525 sold by Lubrizol, Polyurethanes-1 and -6 sold under the trade name Luviset® P.U.R. by BASF, Hydrogenated Dimer Dilinoleyl/Dimethylcarbonate Copolymer sold under the trade name of Cosmedia® DC by Cognis.

Moreover, the pH-value of the second aqueous phase preferably ranges between 8 and 11 and can be adjusted, for example, by using alkaline solutions such as potassium or sodium hydroxide solutions. Even more preferred the pH of the second aqueous phase is greater than 9 and less than 11. If the pH-value is within this range, the reaction of the crosslinking agent(s), i.e., the isocyanate(s) with the (amino)saccharide(s) in the organic phase is facilitated. In the case of amine containing ingredients, high pH-values, i.e., pH-values higher than 8 to 9 cause a conversion of amine hydrochloride to free amine (—NH₂, NH—). As amine hydrochloride groups react slower with isocyanates than free amine groups, higher pH-values are generally preferred. When saccharide-based building blocks are used, likewise, pH-values higher than 8 are advantageous. The pH-value can be adjusted correspondingly by addition of alkaline water-soluble hydroxides and/or basic catalysts such as DABCO®.

Subsequently, in step (d) the oil phase and the first aqueous phase are blended to obtain a preliminary oil-in-water emulsion, with finely dispersed and stabilized discrete oil droplets. In this step the oil phase (comprising the isocyanate component and active ingredients and optionally additional oil components) and the first aqueous phase (comprising the capsule formation aid(s)) are mixed to form finely spread oil droplets within the continuous aqueous phase.

Emulsions, comprising two phases, can be subdivided into two different types. In an oil-in-water (O/W) emulsion oil droplets are dispersed in water. This is the most common type of emulsion. Conversely, a water-in-oil (W/O) emulsion involves water droplets finely dispersed in oil. Within the context of the present invention, the emulsion is an oil-in-water (O/W) emulsion in which the oily phase is dispersed in an aqueous phase. Preferably, the active ingredients are hydrophobic, thus being comprised as core material in the inventive core-shell microcapsules.

Alternatively, it is possible to prepare a corresponding water-in-oil emulsion for the encapsulation of hydrophilic actives.

In order to achieve an interfacial reaction of the isocyanate component(s) and the (amino)saccharide component(s) it is necessary the isocyanate component(s) is/are predominantly water-insoluble while the (amino)saccharide component(s) show water-solubility.

Core-shell capsules are usually produced by fine dispersion of the core material(s) in an aqueous phase. Based on the reaction between isocyanate and saccharide and/or aminosaccharide components in the oil phase near the interface of the aqueous phase and the oil phase the polymeric wall material is formed around the finely dispersed/discrete oil droplets. This results in a suspension containing the as-obtained microcapsules having a shell wall and an oil core. The size of the oil droplets therefore directly determines the size of the subsequently formed capsule cores. High rotating speeds allow for the formation of finely spread discrete and homogeneous oil droplets, resulting in a homogeneous core-shell microparticle sizes.

As indicated above, advantageously stirring is applied throughout the whole preparation process.

Emulsion formation in the case of liquid active ingredients or suspension formation in the case of solid active ingredients according to the present invention, i.e., emulsification or suspension of the internal non-aqueous or oily phase with the external aqueous or hydrophilic phase, takes place under high turbulence or strong shear, whereby the strength of the turbulence or shear determines the diameter of the microcapsules obtained. The production of the microcapsules can be continuous or discontinuous. With increasing viscosity of the aqueous phase or with decreasing viscosity of the oily phase, the size of the resulting capsules usually decreases.

Preferably emulsification, i.e., the formation of the preliminary oil-in-water emulsion preferably takes place by subjecting the mixture to high-speed shearing, for example using an Ultra-Turrax® from IKA® Works at 3 000 rpm to 5 000 rpm for about 20 seconds to about 120 seconds in order to achieve a homogeneous preliminary oil-in-water emulsion with discrete particles homogenous in size. Subsequently, the stirring speed was lowered to 300 rpm to 800 rpm and preferably 600 rpm to 650 rpm in order to prevent the agglomeration of the particles, for example by using an overhead mixer.

Thereby, the temperature is preferably kept within a range of 0° C. to 50° C. and preferably between 20° C. and 40° C.

Once the preliminary oil-in-water emulsion is prepared, it is blended with the second aqueous phase containing the at least one saccharide and/or at least one aminosaccharide and/or at least one thiosaccharide each independently having less than 20 monomeric units, preferably 15 or less monomeric units, even more preferred 10 or less monomeric units as defined above, in order to form polyurethane and/or polyurea and/or polythiourethane-based linkages by reaction of the at least two functional groups, i.e. the hydroxyl groups and/or amine groups and/or thiol groups, with the isocyanate functional groups of the isocyanate components.

The second aqueous phase is preferably added at a stirring speed of 300 rpm to 800 rpm and preferably 600 rpm to 650 rpm. Preferably, the second aqueous phase is added at a temperature of 0° C. to 50° C. and preferably between 20° C. and 40° C.

In the process according to invention, the at least one saccharide and/or aminosaccharide has at least two functional groups independently selected from the group consisting of: primary and secondary hydroxyl groups (—OH) as well as primary and secondary amine groups (—NH₂, —NH—).

Preferably, the saccharide- and/or aminosaccharide- and/or thiosaccharide-components having at least two functional reactive groups independently selected from the group consisting of: primary and secondary hydroxyl groups (—OH) as well as primary and secondary amine groups (—NH₂, —NH—) or thiol (—SH) groups.

Preferably, in the process for preparing core-shell microcapsules or a slurry comprising core-shell microcapsules or the microcapsules according to the first aspect, the molar or weight ratio, and preferably the molar ratio, of the at least one polyisocyanate having at least two isocyanate groups to the at least one saccharide and/or aminosaccharide and/or at least one thiosaccharide having less than 20 monomeric units and preferably having at least two functional reactive groups independently selected from the group consisting of: primary and secondary hydroxyl groups (—OH) as well as primary and secondary amine groups (—NH₂, —NH—) or thiol (—SH) groups, is in the range of from 1:3 to 1:1, and preferably from 1:3 to 1:2.

If the isocyanate to (amino)saccharide ratio is within the ranges specified above, it is possible to achieve efficient crosslinking and thus highly stable microcapsules which however still show excellent release properties.

Finally, the microcapsules obtained in step (e) in form of a microcapsule slurry have to be cured.

Once the preliminary oil-in-water emulsion is blended with the second aqueous phase, polyaddition takes place and the crude microcapsules encapsulating the actives are formed. It is understood that the capsule formation aid(s) is/are either incorporated into the capsule shell or bond to its surface. At that time the microcapsules show insufficient stability in the dispersion, therefore a final curing step is necessary: After the completion of the crosslinking process, the as-prepared microcapsules are present as raw microcapsules in the form of an aqueous dispersion with still soft and flexible capsule shells or capsule walls. Subsequently, it is therefore necessary to subject the microcapsules to an additional curing process in order to cure, i.e., harden the still soft, flexible and unstable single-layer microcapsule shells or walls and thus to provide sufficient stability by consuming the isocyanate component and thereby growing the polymer. Typically curing takes place by treating the obtained dispersion at elevated temperatures of from about 50° C. to about 90° C. over a period of from about 1 hour to about 12 hours. In order to avoid aggregation and thus clumping of the crude microcapsules it is advantageous to agitate the dispersion continuously during the curing process at 300 rpm to 1000 rpm using non-high shear blades. Preferably, the stirring speed is about 650 rpm during the curing process.

If desired the solvent can be removed to obtain the “pure” capsules, which typically show an average diameter of from about 5 microns to about 50 microns.

Consequently, the process for preparing biobased core-shell microcapsules by preparing a slurry optionally comprise a subsequently additional step of isolating the microcapsules as such preferably in dry form from the slurry/dispersion (step (g)).

This can be achieved by, for example, filtration. Further common techniques suitable for this purpose are, for example, filtration or spray drying, freeze drying, or vacuum drying to remove the solvent(s) and to obtain the isolated microcapsules. Also, centrifugation and subsequent drying might be an option.

Optionally, the process comprises an additional step (f2) instead of the isolation of the microcapsule after step (f), wherein one or more suspension aids or structuring aids chosen from natural gums (e.g., xanthan gum, gellan gum, diutan gum, cellulose gum) are added as thickening agents to provide for an increased suspension stability.

Thereby the thickening agent(s) is/are preferably added while stirring at approximately 1200 rpm to 1500 rpm for 1 minute to 5 minutes before lowering the stirring speed to less than 1000 rpm, preferably about 850 rpm. Thereby, the thickener or structuring agent is added to provide slurry stability against “creaming” or settling of the particles.

Preferably, subsequently, the as obtained capsules are cured again in an additional step, optionally followed by isolation of the microcapsules from the slurry as described above.

In addition, preferably catalyst(s) can be added in order to promote the reaction between the (amino)saccharide building blocks and the isocyanate crosslinkers, in particular with regard to the formation of polyurethane-based structures. Suitable catalysts are for example metal-ligand catalyst based on tin, zinc, bismuth or a tertiary amine such as DABCO® (1,4-diazabicyclo[2.2.2]octane; Air Products & Chemicals, Inc; Supplier: Sigma-Aldrich Corp. St. Louis, MO, USA.).

Preferably, the catalyst(s) are added to the oil and/or one or both of the aqueous phases and/or directly to the (preliminary) oil-in-water emulsion. Preferably, approximately 0.03% to 0.1% by weight of a metal-based catalyst (bismuth neodecanoate) are added to the organic/oil phase and additionally approximately 0.03% to 0.1% by weight of a tertiary amine (such as DABCO®) to one of the aqueous phases or directly to the (preliminary) oil-in-water emulsion for synergistic catalysis. Preferably, bismuth neodecanoate is used as the catalyst as this compound preferred due to its low toxicity compared other metal-ligand catalysts.

All in all, the process described herein allows for the preparation of stable and well-performing biobased or saccharide-based core-shell microcapsules which, due to the more efficient crosslinking, allow for significant savings in shell material and thus make it possible to further reduce the required isocyanate content compared to state-of-the-art capsules without adversely affecting the stability or performance of the capsules. In addition, the capsules produced in this way have a very good base compatibility and increased biodegradability and are therefore particularly suitable for use in various consumer goods, especially against the background of the constantly increasing health and environmental awareness of today's society.

Therefore, another object of the present invention is to provide a slurry comprising biobased core-shell microcapsules obtained by the process described above as well as the biobased core-shell microcapsule as such obtained by said process.

In addition, the inventive biobased core-shell microcapsules and/or the inventive slurry comprising the core-shell microcapsules are suitable for the use in the preparation of various consumer products.

Thus, the present invention also pertains to the use of the core-shell microcapsules according to the present invention for preparing consumer products. The core-shell microcapsules of the present invention are suitable for use, without limitation, in the following applications: cosmetics, personal care products, in particular skin cleaning and skin care products, shampoos, rinse-off conditioners, deodorants, antiperspirants, body lotions, textile care products and homecare/household products, in particular liquid detergents, all-purpose cleaners, laundry and cleaning agents, fabric softeners, scent boosters, fragrance enhancers and pharmaceuticals.

Finally, the present invention also pertains to preferably perfumed consumer products comprising the core-shell microcapsules according to the invention or the microcapsules slurry according to the invention, wherein the consumer product is selected amongst others from the group comprising or consisting of: cosmetics, personal care products, in particular skin cleaning and skin care products, shampoos, rinse-off conditioners, deodorants, antiperspirants, body lotions, textile care products and homecare/household products, in particular liquid detergents, all-purpose cleaners, laundry and cleaning agents, fabric softeners, scent boosters, fragrance enhancers, as well as pharmaceuticals.

Examples of personal-care products include shampoos, rinses, hair conditioners, soaps, creams, body washes such as shower or bath salts, body soaps, solid and liquid soaps in general, body liquids, mousses, oils or gels, hygiene products, cosmetic preparations in general, body lotions, leave-on personal care applications including hair refreshers and lotions, personal cleaners or sanitizers, pre-shave products, splash colognes and perfumed refreshing wipes, shower gels, shaving soaps, shaving foams, bath oils, cosmetic emulsions such as skin creams and lotions, face creams and lotions, sun protection creams and lotions, after sun creams and lotions, hand creams and lotions, foot creams and lotions, depilatory creams and lotions, aftershave creams and lotions, tanning creams and lotions, hair care products such as hair sprays, hair gels, hair lotions, hair conditioners, permanent and semi-permanent hair dyes, hair deformers such as cold waves and hair straighteners, hair tonics, hair creams and lotions, deodorants and antiperspirants such as armpit sprays, roll-ons, deodorant sticks, deocreams or decorative cosmetic products. Rinse off products may be liquids, solids, pastes, or gels, of any physical form.

Examples of homecare products include solid or liquid detergents, all-purpose cleaners, fabric softeners and refreshers, ironing waters and detergents, softener and drier sheets, among which liquid, powder and tablet detergents, scent-boosters and fabric softeners are preferred. Further examples are floor cleaners, window glass cleaners, dishwashing detergents, bathroom and sanitary cleaners, scouring lotions, solid and liquid WC-cleaners, powdered and foamed carpet cleaners, liquid washing agents, and powdery washing agents for washing dishes or for cleaning various surfaces, laundry pretreatment agents such as bleaching agents, soaking agents and stain removers, fabric softener, fabric refreshers, washing soaps, washing tablets, disinfectants, surface disinfectants and air fresheners in liquid, gel-like or on a solid carrier applied form, aerosol sprays, waxes and polishes such as furniture polishes, floor waxes and, shoe polishes.

Preferred laundry/textile care products include, in particular, textile care products and detergent-containing preparations, such as powder and liquid detergents and fabric softeners.

The present core-shell microcapsules allow for the efficient encapsulation of a broad range of active ingredients such as fragrances and thus offers the possibility of reducing or completely preventing interactions in the perfumed product with other ingredients of the product formulation as well as the evaporation of the potentially slightly volatile constituents. The use of microcapsules allows for a targeted release the constituent(s) under precisely defined conditions. Thus, for example, in the case of microcapsules which contain fragrant substances, it is possible to enclose the fragrances, which are generally sensitive to environmental conditions (e.g., oxidative effects caused by air or other ingredients of the product formulation), so that they are stable in storage. Only at the moment of the desired fragrance release, the microcapsules are broken by applying a specific signal or trigger (such as mechanical stress) efficiently releasing the fragrance and thus showing a good sensory performance as defined herein. The inventive capsules allow for a controlled release of the active ingredients (without losses upon storage) upon breaking of the capsule shell and simultaneously provide sufficient stability to the capsules for long periods of time while showing an increased biodegradability compared to capsules of the state of the art.

### EXAMPLES

### Preparation of Core-Shell Microcapsules

The following encapsulated particles (core-shell microcapsules) were prepared as described below. Based on the preparations described below corresponding microcapsule slurries comprising a plurality of biobased/(amino)saccharide-based core-shell microcapsules dispersed in an aqueous phase were obtained showing increased biodegradability compared to fully synthetic (non-biobased) state-of-the-art microcapsules and having excellent release properties and capsule stabilities even upon long-term storage.

Table 1 summarizes the equivalent weight and functional group content of the isocyanate components used within the context of the present invention and the inventive examples.

TABLE 1
Isocyanate components of the isocyanate components used in the
following examples.
$Equivalent weight = \frac{42.06 \times 100}{\% NCO}$

| Isocyanate component | % NCO (Supplier) | Equivalent weight |
|---|---|---|
| Takenate 600 (aliphatic) | 43.3 | 97.1 |
| Stabio 370N (aliphatic) | 25.0 | 168.2 |
| Bayhydur 305 (aliphatic) | 16.2 ± 0.4 | 259.6 |
| Desmodur N-3400 (aliphatic) | 21.8 ± 0.7 | 192.9 |
| Mondur M (aromatic) | 33.6 | 125.18 |

Table 2 summarizes the relative weight and molar ratios of the isocyanate compounds used within the context of the present invention and the examples.

TABLE 2
Relative weight ratio and molar ratio of the isocyanate compounds.

| | Relative weight | Relative molar |
|---|---|---|
| Isocyanate component | ratio | ratio |
| Desmodur N-3400/Mondur M | 80/20 | 72.2/27.8 |
| (aliphat./aromat.) | | |
| Desmodur N-3400/Mondur M | 86/14 | 80.1/19.9 |
| (aliphat./aromat.) | | |
| Desmodur N-3400/Mondur M | 90/10 | 85.4/14.6 |
| (aliphat./aromat.) | | |
| Stabio 370N/Takenate 600 | 50/50 | 36.6/63.4 |
| (aliphat./aliphat.) | | |
| Bayhydur 305/Takenate 600 | 90/10 | 77.1/22.9 |
| (aliphat./aliphat.) | | |
| Bayhydur 305/Stabio 370N/ | 50/20/30 | 33.4/30.9/35.7 |
| Takenate 600 | | |
| (aliphat./aliphat./aliphat.) | | |

### Example 1: Glucosamine-Based Microcapsules I

Microcapsules according to the present invention were prepared using glucosamine as the multi-functional nucleophile and polyvinyl alcohol (PVA, Selvol™ 523, Sekisui Specialty Chemicals, Japan) as the capsule formation aid.

More specifically, 210 g of a fragrance material, here TomCap® (Symrise, Teterboro, NJ), were weighed out in a 250 ml-beaker and combined with 6.29 g of Desmodur® N-3400 isocyanate monomer (hexamethylene diisocyanate uretidione, HDI-uretidione; aliphatic polyisocyanate; equivalent weight: 193) and 1.57 g of Mondur® M flakes (monomeric diphenylmethane-4,4′-diisocyanate; aromatic polyisocyanate; equivalent weight: 125.2), both from Covestro Corporation, to form an oil phase comprising the aliphatic and aromatic polyisocyanates in a relative weight ratio of 80:20 or a relative molar ratio of 72.3:27.7, respectively. Subsequently, 0.3 g of an oil soluble catalyst (bismuth neodecanoate, Aldrich Chemical Corporation, St. Louis, MO, USA) were added. In a separate 800 ml-beaker, a solution (322 g) containing 32.2 g of polyvinyl alcohol (PVA, Selvol™ 523, Sekisui Specialty Chemicals, Japan) in water was prepared to form the first aqueous phase. The oil phase was then emulsified into the aqueous phase to obtain an oil-in-water fragrance emulsion having an average particle size ranging from 5 to 50 microns by applying shear forces (Ultra Turrax®, T-50, commercially available from IKA® Werke, Staufen, Germany) by stirring at 3500 rpm for about 20 to 60 seconds in the presence of 0.3 g of DABCO® (1,4-diazabicyclo[2.2.2]octane, Aldrich Chemical Corporation, St. Louis, MO, USA), i.e. the catalyst is added after the emulsification to the preliminary oil-in-water emulsion. The particle size of the finely dispersed oil particles was measured using a Mastersizer® 3000 particle size analyzer (Malvern Instruments, 117 Flanders Road, Westborough, MA, USA). Preferably, the median particle size of the oil droplets finely dispersed within the aqueous phase is ranging from 20 μm to 30 μm.

The as obtained preliminary oil-in-water emulsion was placed within an overhead mixer and agitated at 600 rpm while 60 g of a 10% glucosamine solution (second aqueous phase) were added incrementally. The used glucosamine solution was prepared by dissolving 6 g of glucosamine HCL (Biosynth Carbosynth®, United Kingdom) in 54 g of deionized water. The pH was adjusted to a value from approximately 9.5 to 10.5 by adding potassium hydroxide. The resulting capsule slurry was cured by heat at 70° C. for at least 3 hours. The microparticles in the suspension will separate over time due to density differences between the aqueous phase (water) and the microparticle density. In order to avoid this process, a structuring agent is added to provide solution viscosity. Therefore, finally, 0.60 g of Kelco-Vis™ DG (diutan gum, CP Kelco Inc.) were added in order to stabilize the as-obtained microcapsules within the aqueous phase while stirring the slurry at 1200 rpm for approximately 2 to 4 minutes before lowering the stirring speed to 850 rpm and subsequent curing for one more hour.

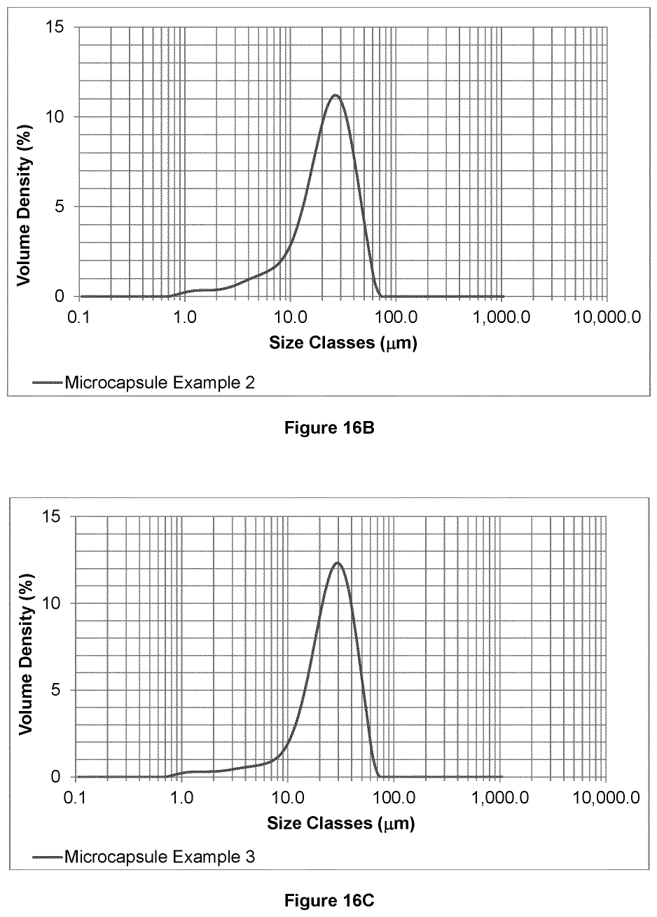

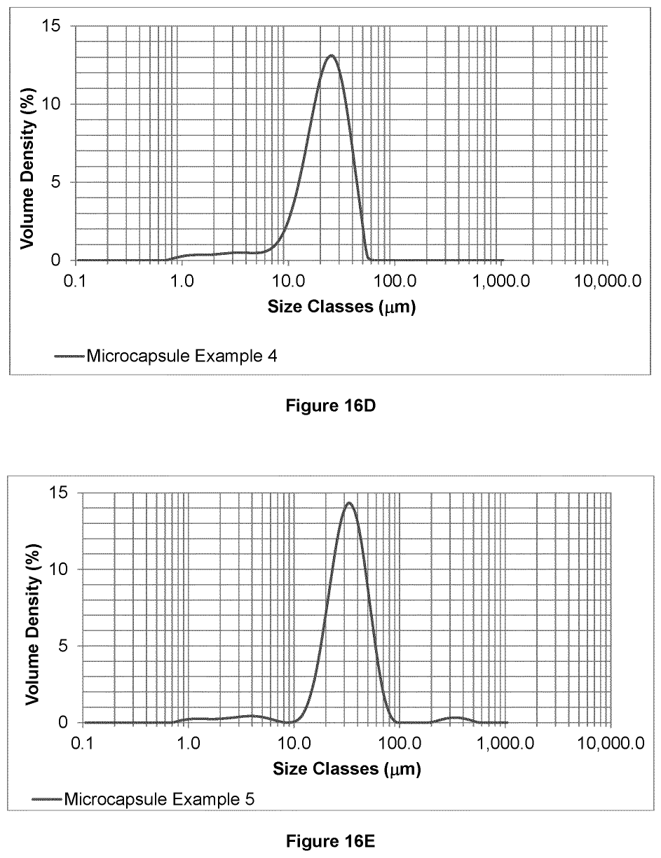

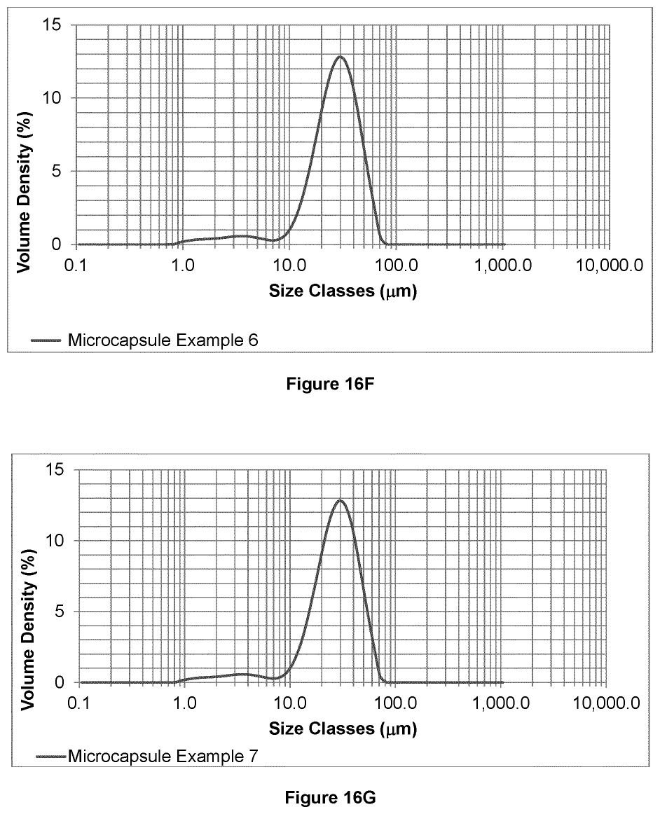

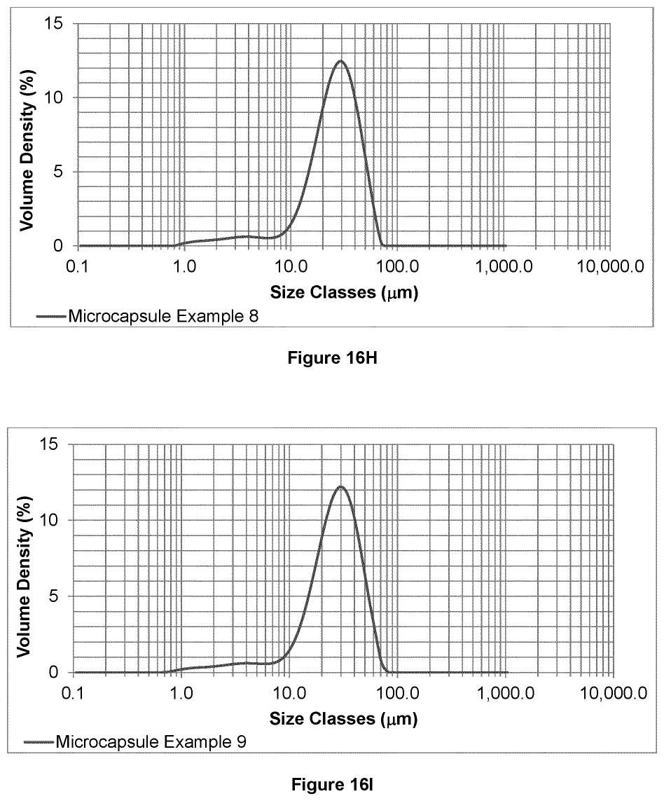

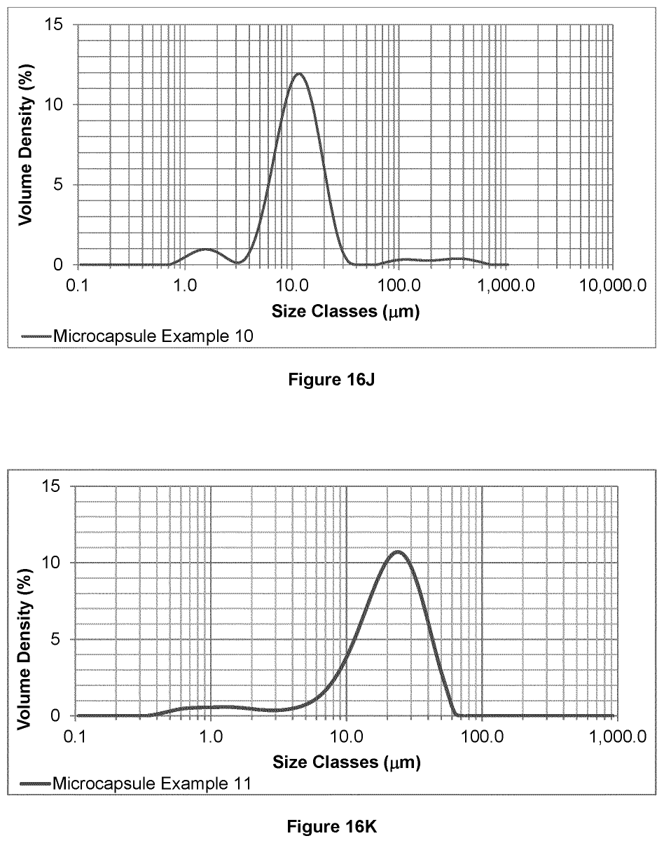

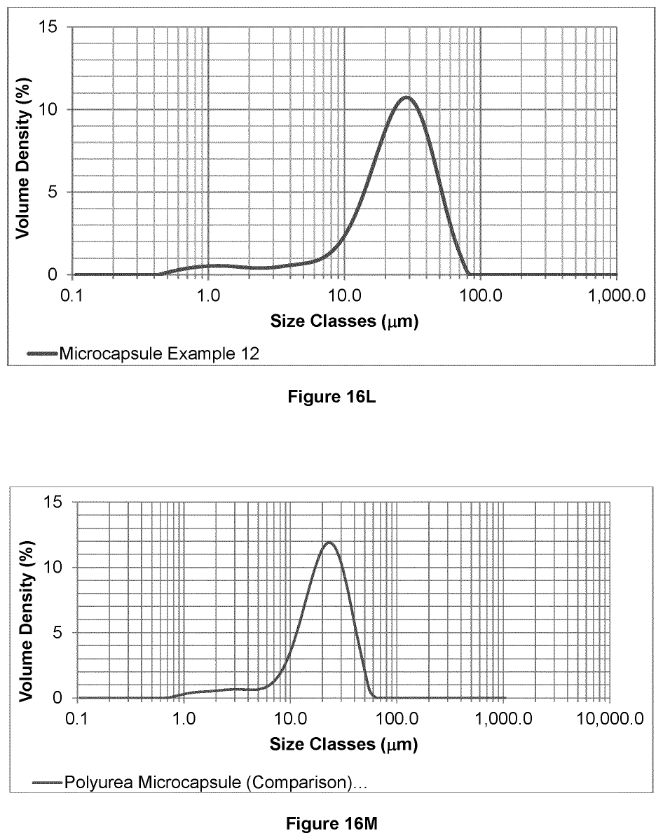

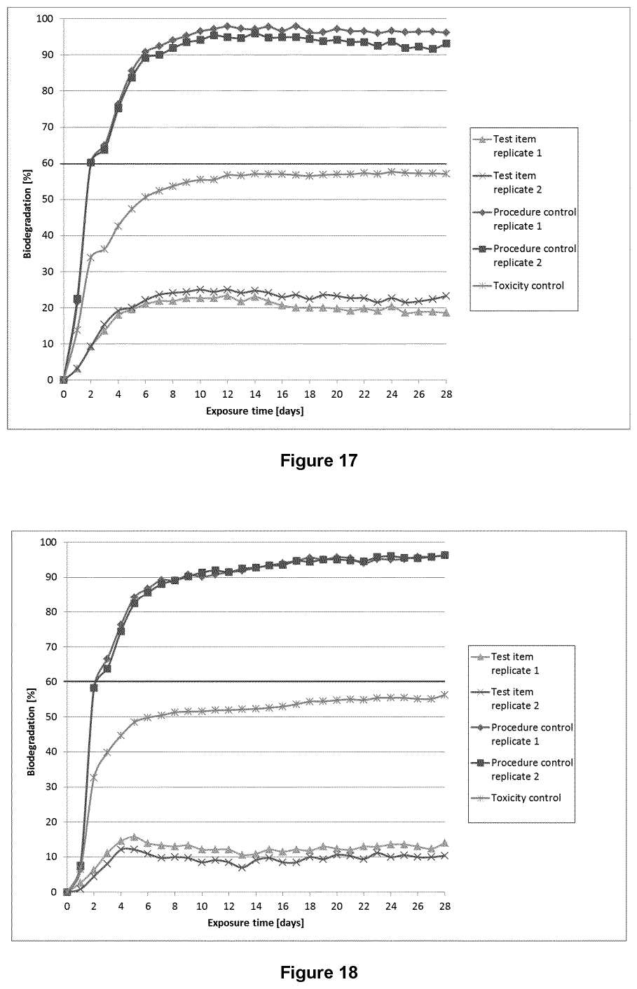

The as-obtained microcapsules have a rather narrow particle size distribution range within the aqueous phase indicating biobased core-shell microcapsules having uniform particle sizes. The core-shell microcapsules have a median particle size by volume (Dv(50)) of 25.3 μm (see FIG. 16A).

Additionally, the resulting microcapsules show high mechanical and chemical stabilities e.g., in product formulations (fabric softener) and excellent release properties, clearly outperforming non-biobased state-of-the-art microcapsules (see FIGS. 1A and 1B). Moreover, the as-obtained microcapsules show significantly enhanced biodegradability properties compared to the state-of-the-art microcapsules which are not biodegradable at all (see FIGS. 13 to 15).

### Example 2: Chito-Oligosaccharide-Based Microcapsules I

Microcapsules according to the present invention were prepared using a chitosan oligosaccharide (chito-oligosaccharide) as the multi-functional nucleophile and polyvinyl alcohol (PVA, Selvol™ 523, Sekisui Specialty Chemicals, Japan) as the capsule formation aid.

More specifically, 210 g of a fragrance material, here TomCap® (Symrise, Teterboro, NJ), were weighed out in a 250 ml-beaker and combined with 6.29 g of Desmodur® N-3400 isocyanate monomer (hexamethylene diisocyanate uretidione, HDI-uretidione; aliphatic polyisocyanate; equivalent weight: 193) and 1.57 g Mondur® M flakes (monomeric diphenylmethane-4,4′-diisocyanate; aromatic polyisocyanate; equivalent weight: 125.2), both from Covestro Corporation, to form an oil phase comprising the aliphatic and aromatic polyisocyanates in a relative weight ratio of 80:20 or a relative molar ratio of 72.3:27.7, respectively. Subsequently, 0.3 g of an oil soluble catalyst (bismuth neodecanoate, Aldrich Chemical Corporation) were added. In a separate 800 ml-beaker, a solution (302 g) containing 3.02 g of polyvinyl alcohol (PVA, Selvol™ 523, Sekisui Specialty Chemicals, Japan) in water was prepared to form the first aqueous phase. The oil phase was then emulsified into the aqueous phase to obtain an oil-in-water fragrance emulsion having an average particle size ranging from 5 to 50 microns by applying shear forces (Ultra Turrax®, T-50, commercially available from IKA® Werke, Staufen, Germany) by stirring at 3500 rpm for about 20 to 60 seconds in the presence of 0.3 g of DABCO® (1,4-diazabicyclo[2.2.2]octane, Aldrich Chemical Corporation, St. Louis, MO, USA). The particle size of the finely dispersed oil particles was measured using a Mastersizer® 3000 particle size analyzer (Malvern Instruments, 117 Flanders Road, Westborough, MA, USA).

The as-obtained preliminary oil-in-water emulsion was placed within an overhead mixer and agitated at 600 rpm while 80 g of a 10% chitosan oligosaccharide solution (second aqueous phase) were added incrementally. The chitosan oligosaccharide solution was prepared by dissolving 8 g of chitosan oligosaccharide HCL (molecular weight <3000 Da, Biosynth Carbosynth®, United Kingdom) in 72 g of deionized water. The pH-value was adjusted to approximately 10 to 11 by adding potassium hydroxide. The resulting capsule slurry was cured by heat at 70° C. for at least 3 hours. Finally, 0.60 g of Kelco-Vis™ DG (diutan gum, CP Kelco Inc.) were added while stirring the slurry at 1200 rpm for approximately 2 to 4 minutes before lowering the stirring speed to 850 rpm and subsequent curing for one more hour.

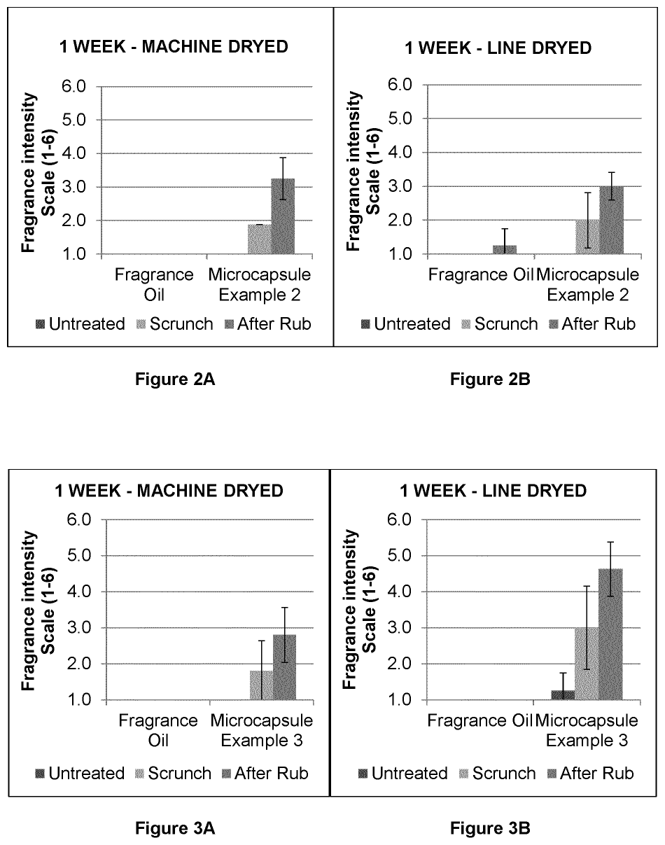

The final core-shell microcapsules have a median particle size by volume (Dv(50)) of 22.9 μm within the slurry (see FIG. 16B) and show good stabilities and release properties even upon machine drying and aging in a softener formulation (see FIGS. 2A and 2B).

### Example 3: Chito-Oligosaccharide-Based Microcapsules II

Microcapsules according to the present invention were prepared using a chitosan oligosaccharide (chito-oligosaccharide) as the multi-functional nucleophile and polyvinyl alcohol (PVA, Selvol™ 523, Sekisui Specialty Chemicals, Japan) as the capsule formation aid.

More specifically, 210 g of a fragrance material, here TomCap® (Symrise, Teterboro, NJ), were weighed out in a 250 ml-beaker and combined with 1.57 g of STABIO™ D-370N (aliphatic polyisocyanurates based on bio-based 1,5-pentamethylene diisocyanate (PDI); equivalent weight: 168.24) and 1.57 g of Takenate™ 600 (1,3-bis(isocyanatomethyl)cyclohexane; aliphatic polyisocyanate; equivalent weight: 97.14), both from Mitsui Chemicals (Japan), to form an oil phase comprising the aliphatic polyisocyanates in a relative weight ratio of 50:50 or a relative molar ratio of 36.6:63.4, respectively. Subsequently, 0.3 g of an oil soluble catalyst (bismuth neodecanoate, Aldrich Chemical Corporation) were added. In a separate 800 ml-beaker, a solution (302 g) containing 3.02 g of polyvinyl alcohol (PVA, Selvol™ 523, Sekisui Specialty Chemicals, Japan) in water was prepared to form the first aqueous phase. The oil phase was then emulsified into the aqueous phase to obtain an oil-in-water fragrance emulsion having an average particle size ranging from 5 to 50 microns by applying shear forces by stirring at 3500 rpm (Ultra Turrax®, T-50, commercially available from IKA® Werke, Staufen, Germany) for about 20 to 60 seconds in the presence of 0.3 g of DABCO® (1,4-diazabicyclo[2.2.2]octane, Aldrich Chemical Corporation). The particle size of the finely dispersed oil particles was measured using a Mastersizer® 3000 particle size analyzer (Malvern Instruments, 117 Flanders Road, Westborough, MA, USA).

The as-obtained preliminary oil-in-water emulsion was placed within an overhead mixer and agitated at 600 rpm while 80 g of a 10% chitosan oligosaccharide solution were added incrementally. The chitosan oligosaccharide solution was prepared by dissolving 8 g of chitosan oligosaccharide HCL (molecular weight <3000 Da, Biosynth Carbosynth®, United Kingdom) in 72 g of deionized water. The pH-value was adjusted to approximately 9.5 to 10.5 by adding potassium hydroxide. The resulting capsule slurry was cured by heat at 70° C. for at least 3 hours. Finally, 0.60 g of Kelco-Vis™ DG (diutan gum, CP Kelco Inc.) were added while stirring the slurry at 1200 rpm for approximately 2 to 4 minutes before lowering the stirring speed to 850 rpm and subsequent curing for an additional hour.

The final core-shell microcapsules have a median particle size by volume (Dv(50)) of 25.9 μm within the slurry (see FIG. 16C) and show excellent release properties especially after line drying although aged in a fabric softener solution for one week (see FIGS. 3A and 3B). In this particular case, it seems that the increased amount of aliphatic isocyanates seems to positively influence the stability compared to Example 2.

### Example 4: Chito-Oligosaccharide and Glucosamine-Based Microcapsules I

Microcapsules according to the present invention were prepared using glucosamine and a chitosan oligosaccharide as the multi-functional nucleophiles and polyvinyl alcohol (PVA) (Selvol™ 523, Sekisui Specialty Chemicals, Japan) as the capsule formation aid.

More specifically, 210 g of a fragrance material, here TomCap® (Symrise, Teterboro, NJ), were weighed out in a 250 ml-beaker and combined with 3.93 g of STABiO™ D-370N (aliphatic polyisocyanurates based on bio-based 1,5-pentamethylene diisocyanate (PDI); equivalent weight: 168.24) and 3.93 g of Takenate™ 600 (1,3-bis(isocyanatomethyl)cyclohexane; aliphatic polyisocyanate; equivalent weight: 97.14), both from Mitsui Chemicals (Japan), to form an oil phase comprising the aliphatic polyisocyanates in a relative weight ratio of 50:50 or a relative molar ratio of 36.6:63.4, respectively. Subsequently, 0.3 g of an oil soluble catalyst (bismuth neodecanoate, Aldrich Chemical Corporation) were added. In a separate 800 ml-beaker, a solution (302 g) containing 3.02 g of polyvinyl alcohol (PVA, Selvol™ 523, Sekisui Specialty Chemicals, Japan) in water was prepared to form the first aqueous phase. The oil phase was then emulsified into the aqueous phase to obtain an oil-in-water fragrance emulsion having an average particle size ranging from 5 to 50 microns by applying shear forces (Ultra Turrax®, T-50, commercially available from IKA® Werke, Staufen, Germany) by stirring at 3500 rpm for about 20 to 60 seconds in the presence of 0.3 g of DABCO® (1,4-diazabicyclo[2.2.2]octane, Aldrich Chemical Corporation). The particle size of the finely dispersed oil particles was measured using a Mastersizer® 3000 particle size analyzer (Malvern Instruments, 117 Flanders Road, Westborough, MA, USA).

The as-obtained preliminary oil-in-water emulsion was placed within an overhead mixer and agitated at 600 rpm while 80 g of a solution mixture of 5% chitosan oligosaccharide (molecular weight <3000 Da; corresponding to approximately 12 to 15 monomeric units) and 5% glucosamine (both from Biosynth Carbosynth®, United Kingdom) were added incrementally. The mixed chitosan oligosaccharide/glucosamine solution was prepared by dissolving 4 g chitosan oligosaccharide and 4 g glucosamine HCL in 72 g of deionized water. The pH was adjusted to 10-11 by adding potassium hydroxide. The resulting capsule slurry was cured at 70° C. for at least 3 hours. Finally, 0.60 g of Kelco-Vis™ DG (diutan gum, CP Kelco Inc.) were added while stirring the slurry at 1200 rpm for approximately 2 to 4 minutes before lowering the stirring speed to 850 rpm and subsequent curing for an additional hour.

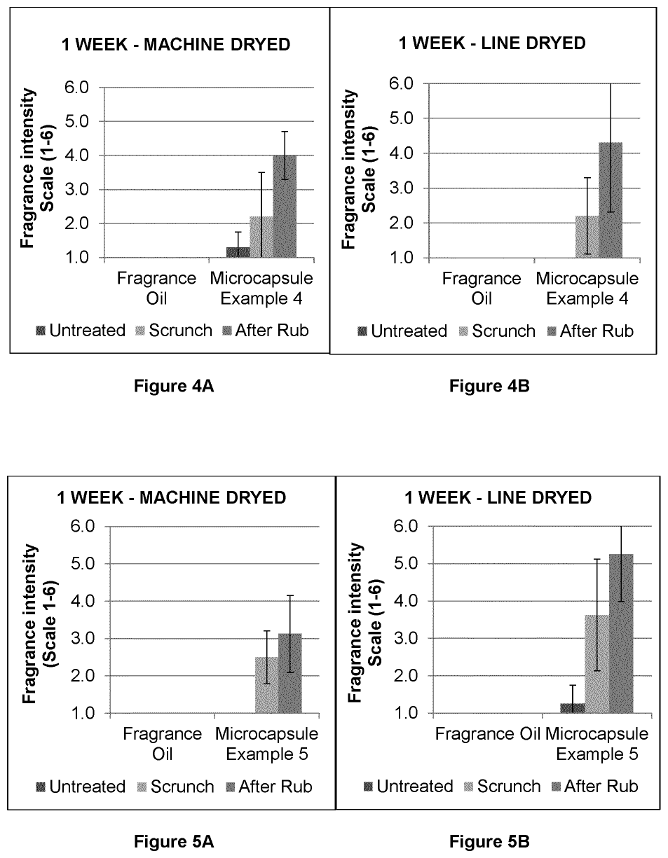

The final core-shell microcapsules have a median particle size by volume (Dv(50)) of 22.6 μm within the slurry (see FIG. 16D) and show excellent performances (release properties) after drying as well as high stabilities within the formulation and during the drying process (see FIGS. 4A and 4B).

### Example 5: Glucosamine-Based Microcapsules II

Microcapsules according to the present invention were prepared using glucosamine (Biosynth Carbosynth®, United Kingdom) as the multi-functional nucleophile and Capsul® starch (commercially available from Ingredion Inc.) as the capsule formation aid.

More specifically, 210 g of a fragrance material, here TomCap® (Symrise, Teterboro, NJ), were weighed out in a 250 ml-beaker and combined with 6.29 g of Desmodur® N-3400 isocyanate monomer (hexamethylene diisocyanate uretidione, HDI-uretidione; aliphatic polyisocyanate; equivalent weight: 193) and 1.57 g of Mondur® M flakes (monomeric diphenylmethane-4,4′-diisocyanate; aromatic polyisocyanate; equivalent weight: 125.2), both from Covestro Corporation, to form an oil phase comprising the aliphatic and aromatic polyisocyanates in a relative weight ratio of 80:20 or a relative molar ratio of 72.3:27.7, respectively. Subsequently, 0.6 g of an oil soluble catalyst (bismuth neodecanoate, Aldrich Chemical Corporation) were added. In a separate 800 ml-beaker, a solution (322 g) containing 6.44 g of octenyl succinic anhydride (OSA) starch (Capsul®, Ingredion Inc.) in water was prepared to form the first aqueous phase. The oil phase was then emulsified into the aqueous phase to obtain an oil-in-water fragrance emulsion having an average particle size ranging from 5 to 50 microns by applying shear forces by stirring at 3500 rpm (Ultra Turrax®, T-50, commercially available from IKA® Werke Staufen, Germany) for about 20 to 60 seconds in the presence of 0.6 g of DABCO® (1,4-diazabicyclo[2.2.2]octane, Aldrich Chemical Corporation). The particle size of the finely dispersed oil particles was measured using a Mastersizer® 3000 particle size analyzer (Malvern Instruments, 117 Flanders Road, Westborough, MA, USA).

The as-obtained preliminary oil-in-water emulsion was placed within an overhead mixer and agitated at 600 rpm while 60 g of a 10% glucosamine solution were added incrementally. The glucosamine solution was prepared by dissolving 6 g of glucosamine HCL (Biosynth Carbosynth®, United Kingdom) in 54 g of deionized water. The pH was adjusted to 10-11 by adding potassium hydroxide. The resulting capsule slurry was cured at 70° C. for at least 3 hours. Finally, 1.8 g Keltrol® (xanthan gum, CP Kelco Inc.) was added while stirring the slurry at 1200 rpm for approximately 2 to 4 minutes before lowering the stirring speed to 850 rpm and subsequent curing for an additional hour.

The final core-shell microcapsules have a median particle size by volume (Dv(50)) of 32.1 μm within the slurry (see FIG. 16E) and show outstanding mechanical and chemical stabilities as well as capsule performances especially after line drying. Using starch as the capsule formation aid, i.e., a biobased capsule formation aid, seems to have a positive influence on the capsule stability and release properties of the inventive core-shell microcapsules (see FIGS. 5A and 5B).

### Example 6: Maltodextrin-Based Microcapsules I

Microcapsules according to the present invention were prepared using maltodextrin DE 8 as the multi-functional nucleophile and polyvinyl alcohol (PVA, Selvol™ 523, Sekisui Specialty Chemicals, Japan) as the capsule formation aid.

More specifically, 210 g of a fragrance material, here TomCap® (Symrise, Teterboro, NJ), were weighed out in a 250 ml-beaker and combined with 6.29 g of Desmodur® N-3400 isocyanate monomer (hexamethylene diisocyanate uretidione, HDI-uretidione; aliphatic polyisocyanate; equivalent weight: 193) and 1.57 g Mondur® M flakes (monomeric diphenylmethane-4,4′-diisocyanate; aromatic polyisocyanate; equivalent weight: 125.2), both from Covestro Corporation, to form an oil phase comprising the aliphatic and aromatic polyisocyanates in a relative weight ratio of 80:20 or a relative molar ratio of 72.3:27.7, respectively. Subsequently, 0.7 g of an oil soluble catalyst (bismuth neodecanoate, Aldrich Chemical Corporation) were added. In a separate 800 ml-beaker, a 1% PVA solution (approx. 241 g) (i.e., the PVA solution having a concentration of 1%) (Selvol™ 523, Sekisui Specialty Chemicals, Japan) was prepared to form the first aqueous phase. Said solution was prepared by mixing 6.5 g of a 10% PVA solution (thus corresponding to 0.65 g of pure PVA) with 235.2 g of deionized water. The as-prepared solution corresponds to 0.25% of the total addition of PVA within the conduct of the experiment (i.e., one fourth of the total PVA addition). The oil phase was then emulsified into the said aqueous phase to obtain an oil-in-water fragrance emulsion having an average particle size ranging from 4 to 50 microns by applying shear forces by stirring at 3500 rpm (Ultra Turrax®, T-50, commercially available from IKA® Werke, Staufen, Germany) for about 20 to 60 seconds. The particle size of the finely dispersed oil particles was measured using a Mastersizer® 3000 particle size analyzer (Malvern Instruments, 117 Flanders Road, Westborough, MA, USA).

The as-obtained preliminary oil-in-water emulsion was placed within an overhead mixer and agitated at 650 rpm while the remaining 0.75% PVA solution (i.e., the remaining three fourths of the total addition of 1% PVA containing approximately 1.96 g of PVA in deionized water) (Selvol™ 523, Sekisui Specialty Chemicals, Japan) were added as well as 0.7 g of the oil soluble catalyst (bismuth neodecanoate, Aldrich Chemical Corporation) and 60 g of a 10% maltodextrin DE 8 solution. In total 2.61 g of PVA were added. The maltodextrin DE 8 solution was prepared by dissolving 6 g of maltodextrin having a dextrose equivalent (DE) of 8 in 54 g of deionized water. The resulting capsule slurry was cured at 70° C. for at least 30 minutes. Thereafter, additional 60 g of a 10% maltodextrin DE 8 solution were added, which was prepared in the same manner as previously described. The slurry was again cured at 70° C. for another 30 minutes. Finally, 0.60 g of Kelco-Vis™ DG (diutan gum, CP Kelco Inc.) were added while stirring the slurry at 1200 rpm for approximately 2 to 4 minutes before lowering the stirring speed to 850 rpm and raising the temperature to 80° C. for one more hour.

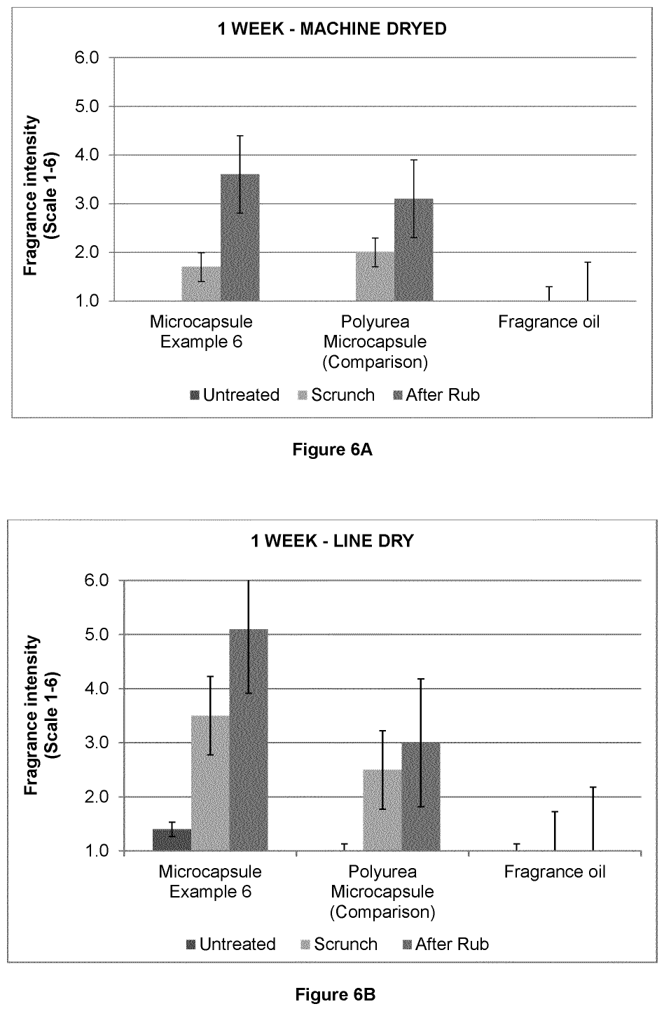

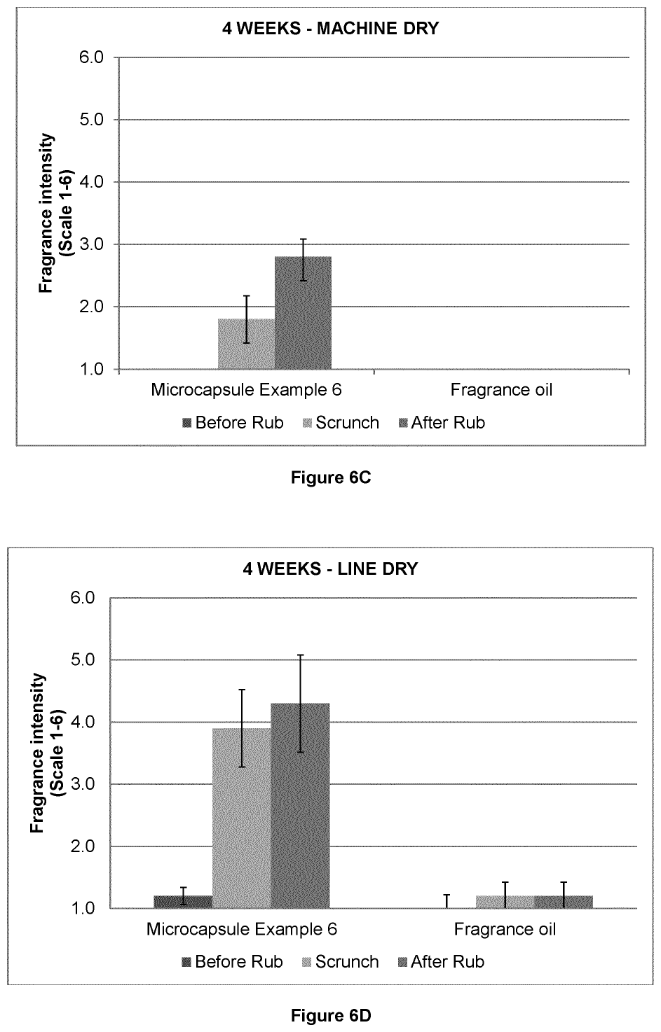

The as-obtained microcapsules have a narrow particle size distribution range within the aqueous phase indicating biobased core-shell microcapsules having uniform particle sizes. The core-shell microcapsules have a median particle size by volume (Dv(50)) of 27.6 μm (see FIG. 16F). The core-shell microcapsules according to Example 6 show considerably improved stabilities as well as release properties compared to state-of-the-art non-biobased microcapsules according to Comparative Example 13 (see FIGS. 6A and 6B). Moreover, the biodegradability is improved compared to said fully synthetic state-of-the-art microcapsules (see FIG. 17).

### Example 7: Maltodextrin-Based Microcapsules II

Microcapsules according to the present invention were prepared using maltodextrin DE 8 as the multi-functional nucleophile and polyvinyl alcohol (PVA, Selvol™ 523, Sekisui Specialty Chemicals, Japan) as the capsule formation aid.

More specifically, 210 g of a fragrance material, here TomCap® (Symrise, Teterboro, NJ), were weighed out in a 250 ml-beaker and combined with 7.07 g of Bayhydur® 305 isocyanate monomer (hydrophilic aliphatic polyisocyanate based on hexamethylene diisocyanate (HDI), Covestro Corporation; equivalent weight: 259.6) and 0.785 g of Takenate™ 600 (1,3-bis(isocyanatemethyl)cyclohexane; aliphatic polyisocyanate, Mitsui Chemicals; equivalent weight: 97.14) to form an oil phase comprising the aliphatic polyisocyanates in a relative weight ratio of 90:10 or a relative molar ratio of 77.1:22.9, respectively. Subsequently, 0.7 g of an oil soluble catalyst (bismuth neodecanoate, Aldrich Chemical Corporation) were added. In a separate 800 ml-beaker, a 1% PVA solution (241 g) in water was prepared to form the first aqueous phase, corresponding to 0.25% of the total addition of 1% PVA (Selvol™ 523, Sekisui Specialty Chemicals, Japan) (see Example 6). The oil phase was then emulsified into the aqueous phase to obtain an oil-in-water fragrance emulsion having an average particle size ranging from 4 to 50 microns by applying shear forces by stirring at 3500 rpm (Ultra Turrax®, T-50, commercially available from IKA® Werke, Staufen, Germany) for about 20 to 60 seconds. The particle size of the finely dispersed oil particles was measured using a Mastersizer® 3000 particle size analyzer (Malvern Instruments, 117 Flanders Road, Westborough, MA, USA).

The as-obtained preliminary oil-in-water emulsion was placed within an overhead mixer and agitated at 650 rpm while the remaining 0.75% of the PVA addition (1% PVA solution in deionized water) (Selvol™ 523, Sekisui Specialty Chemicals, Japan) were added (see Example 6) as well as 0.7 g of the oil soluble catalyst (bismuth neodecanoate, Aldrich Chemical Corporation) and 60 g of a 10% maltodextrin DE 8 solution. The maltodextrin DE 8 solution was prepared by dissolving 6 g of maltodextrin having a dextrose equivalent (DE) of 8 in 54 g of deionized water. The resulting capsule slurry was cured at 70° C. for at least 30 minutes. Thereafter, additional 60 g of a 10% maltodextrin DE 8 solution were added, which was prepared in the same manner as previously described. The slurry was again cured at 70° C. for another 30 minutes. Finally, 0.60 g of Kelco-Vis™ DG (diutan gum, CP Kelco®) were added while stirring the slurry at 1200 rpm for approximately 2 to 4 minutes before lowering the stirring speed to 850 rpm and raising the temperature to 80° C. for one more hour.

The as-obtained microcapsules have a sharp particle size distribution range within the aqueous phase indicating biobased core-shell microcapsules having uniform particle sizes. The core-shell microcapsules have a median particle size by volume (Dv(50)) of 51.5 μm (see FIG. 16G). Moreover, the biodegradability is improved compared to fully synthetic state-of-the-art microcapsules (see FIG. 18).

### Example 8: Maltodextrin-Based Microcapsules III

Microcapsules according to the present invention were prepared using maltodextrin DE 8 as the multi-functional nucleophile and polyvinyl alcohol (PVA, Selvol™ 523, Sekisui Specialty Chemicals, Japan) as the capsule formation aid.

More specifically, 210 g of a fragrance material, here TomCap® (Symrise, Teterboro, NJ), were weighed out in a 250 ml-beaker and combined with 6.29 g of Desmodur® N-3400 isocyanate monomer (hexamethylene diisocyanate uretidione, HDI-uretidione; aliphatic polyisocyanate; equivalent weight: 193) and 1.57 g Mondur® M flakes (monomeric diphenylmethane-4,4′-diisocyanate; aromatic polyisocyanate; equivalent weight: 125.2), both from Covestro Corporation, to form an oil phase comprising the aliphatic and aromatic polyisocyanates in a relative weight ratio of 80:20 or a relative molar ratio of 72.3:27.7, respectively. Subsequently, 0.7 g of an oil soluble catalyst (bismuth neodecanoate, Aldrich Chemical Corporation) were added. In a separate 800 ml-beaker, a 1% PVA solution (241 g) in water was prepared to form the first aqueous phase, corresponding to 0.25% of the total addition of 1% PVA (Selvol™ 523, Sekisui Specialty Chemicals, Japan) (see previous examples). The oil phase was then emulsified into the aqueous phase to obtain an oil-in-water fragrance emulsion having an average particle size ranging from 4 to 50 microns by applying shear forces by stirring at 3500 rpm (Ultra Turrax®, T-50, commercially available from IKA® Werke, Staufen, Germany) for about 20 to 60 seconds. The particle size of the finely dispersed oil particles was measured using a Mastersizer® 3000 particle size analyzer (Malvern Instruments, 117 Flanders Road, Westborough, MA, USA).

The as-obtained preliminary oil-in-water emulsion was placed within an overhead mixer and agitated at 650 rpm while the remaining 0.75% of the PVA addition (1% PVA solution in deionized water) (Selvol™ 523, Sekisui Specialty Chemicals, Japan) were added as well as 0.7 g of the catalyst DABCO® (1,4-diazabicyclo[2.2.2]octane, Aldrich Chemical Corporation) and 60 g of a 10% maltodextrin DE 8 solution. The maltodextrin DE 8 solution was prepared by dissolving 6 g of maltodextrin having a dextrose equivalent (DE) of 8 in 54 g of deionized water. The resulting capsule slurry was cured at 70° C. for at least 30 minutes. Thereafter, additional 60 g of a 10% maltodextrin DE 8 solution were added, which was prepared in the same manner as previously described. The slurry was again cured at 70° C. for another 30 minutes. Finally, 0.60 g of Kelco-Vis™ DG (diutan gum, CP Kelco Inc.) were added while stirring the slurry at 1200 rpm for approximately 2 to 4 minutes before lowering the stirring speed to 850 rpm and raising the temperature to 80° C. for one more hour.

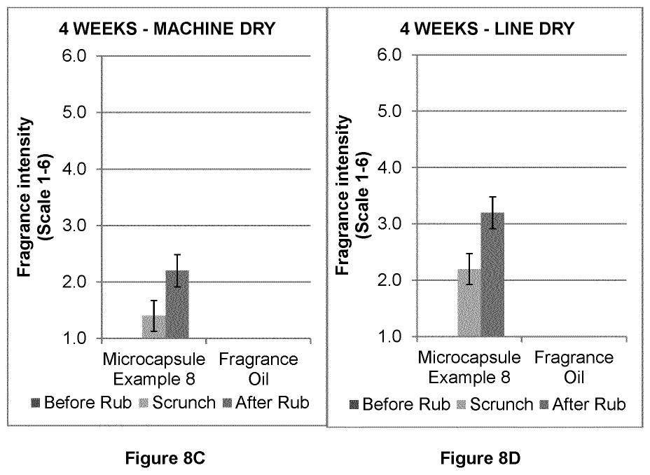

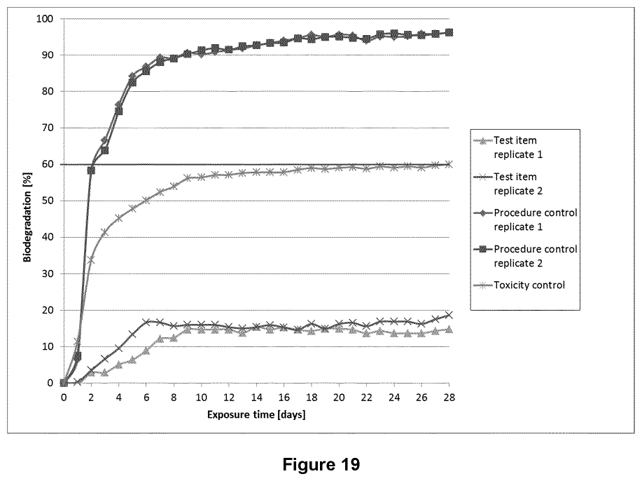

The as-obtained microcapsules have a sharp particle size distribution range within the aqueous phase indicating biobased core-shell microcapsules having uniform particle sizes. The core-shell microcapsules have a median particle size by volume (Dv(50)) of 26.4 μm (see FIG. 16H) and show improved biodegradability (see FIG. 19). Moreover, the inventive capsules show high stabilities and performances even after drying and aging in the product formulation for four weeks (see FIGS. 6A to 6D, 8A to 8D). Examples 6 and 8 differ in the selected catalysts.

### Example 9: Maltodextrin and Glucosamine-Based Microcapsules

Microcapsules according to the present invention were prepared using maltodextrin DE 8 and glucosamine as the multi-functional nucleophiles and polyvinyl alcohol (PVA, Selvol™ 523, Sekisui Specialty Chemicals, Japan) as the capsule formation aid.

More specifically, 210 g of a fragrance material, here TomCap® (Symrise, Teterboro, NJ), were weighed out in a 250 ml-beaker and combined with 6.29 g of Desmodur® N-3400 isocyanate monomer (hexamethylene diisocyanate uretidione, HDI-uretidione; aliphatic polyisocyanate; equivalent weight: 193) and 1.57 g Mondur® M flakes (monomeric diphenylmethane-4,4′-diisocyanate aromatic polyisocyanate; equivalent weight: 125.2), both from Covestro Corporation, to form an oil phase comprising the aliphatic and aromatic polyisocyanates in a relative weight ratio of 80:20 or a relative molar ratio of 72.3:27.7, respectively. Subsequently, 0.5 g of an oil soluble catalyst (bismuth neodecanoate, Aldrich Chemical Corporation) were added. In a separate 800 ml-beaker, a 1% PVA solution (241 g) in water was prepared to form the first aqueous phase, corresponding to 0.25% of the total addition of 1% PVA (Selvol™ 523, Sekisui Specialty Chemicals, Japan) (see previous examples). The oil phase was then emulsified into the aqueous phase to obtain an oil-in-water fragrance emulsion having an average particle size of 4 to 50 microns by applying shear forces by stirring at 3500 rpm (Ultra Turrax®, T-50, commercially available from IKA® Werke, Staufen, Germany) for about 20 to 60 seconds. The particle size of the finely dispersed oil particles was measured using a Mastersizer® 3000 particle size analyzer (Malvern Instruments, 117 Flanders Road, Westborough, MA, USA).

The as-obtained preliminary oil-in-water emulsion was placed within an overhead mixer and agitated at 650 rpm while adding the remaining 75% of the PVA addition (1% PVA solution in deionized water) followed by 0.5 g of the catalyst DABCO® (1,4-diazabicyclo[2.2.2]octane, Aldrich Chemical Corporation) and 60 g of a 10% glucosamine solution. The glucosamine solution was prepared by dissolving 6 of glucosamine HCL (Biosynth Carbosynth®, United Kingdom in 54 g of deionized water followed by an adjustment of the pH-value to 7-8 by addition of KOH. The resulting capsule slurry was cured at 70° C. for 30 minutes followed by the addition of 60 g of a 10% maltodextrin DE 8 solution. The maltodextrin DE 8 solution was prepared by dissolving 6 g of maltodextrin having a dextrose equivalent (DE) of 8 in 54 g of deionized water. The resulting capsule slurry was cured at 70° C. for an additional 30 minutes. Finally, 0.60 g of Kelco-Vis™ DG (diutan gum, CP Kelco Inc.) were added while stirring the slurry at 1200 rpm for approximately 2 to 4 minutes before lowering the stirring speed to 850 rpm and raising the temperature to 80° C. for one more hour. This procedure corresponds to Example 9B.

In parallel a microcapsule slurry was prepared following the process described above, however, with the difference, that 30 g of the corresponding glucosamine solution and 30 g of the corresponding maltodextrin solution were added simultaneously before the first curing step and further 30 g of the glucosamine solution and 30 g of the maltodextrin solution after the first curing step (corresponding to Example 9A).

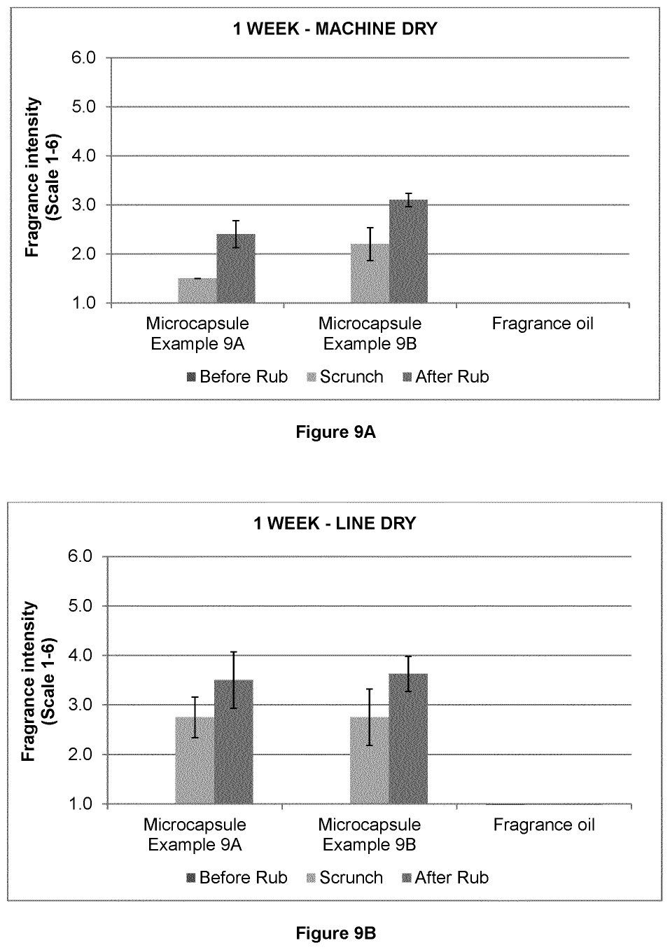

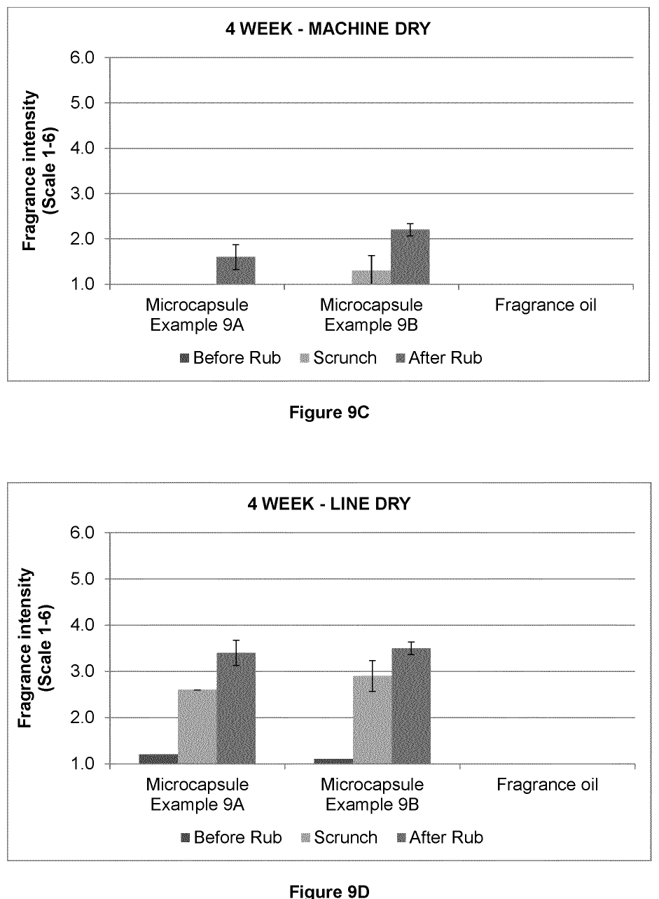

The as-obtained microcapsules have a sharp particle size distribution range within the aqueous phase indicating biobased core-shell microcapsules having uniform particle sizes. The core-shell microcapsules have a median particle size by volume (Dv(50)) of 27.0 μm (see FIG. 16I corresponding to Example 9A) and show satisfactory performances and stabilities (see FIGS. 9A and 9B).

### Example 10: Maltodextrin-Based Microcapsules IV

Microcapsules according to the present invention were prepared using maltodextrin DE 8 as the multi-functional nucleophile and polyvinyl alcohol (PVA, Selvol™ 523, Sekisui Specialty Chemicals, Japan) as the capsule formation aid.

More specifically, 210 g of a fragrance material, here TomCap® (Symrise, Teterboro, NJ), were weighed out in a 250 ml-beaker and combined with 3.93 g of Bayhydur® 305 isocyanate monomer (hydrophilic aliphatic polyisocyanate based on hexamethylene diisocyanate (HDI), Covestro Corporation; equivalent weight: 259.6), 1.57 g of Takenate™ 600 (1,3-bis(isocyanatemethyl)cyclohexane; aliphatic polyisocyanate, Mitsui Chemicals; equivalent weight: 97.14), and 2.36 g of STABIO™ 370-N (1,5-pentamethylene diisocyanate (PDI) based polyisocyanate; aliphatic polyisocyanate, Mitsui chemicals; equivalent weight: 168.24) to form an oil phase comprising the three aliphatic polyisocyanates in a relative weight ratio of 50:20:30 or a relative molar ratio of 33.4:35.7:30.9, respectively. Subsequently, 0.7 g of an oil soluble catalyst (bismuth neodecanoate, Aldrich Chemical Corporation) were added. In a separate 800 ml-beaker, a 1% PVA solution (241 g) in water was prepared to form the first aqueous phase, corresponding to 0.2% of the total addition of 1% PVA (Selvol™ 523, Sekisui Specialty Chemicals, Japan) (see previous examples). The oil phase was then emulsified into the aqueous phase to obtain an oil-in-water fragrance emulsion having an average particle size ranging from 4 to 50 microns by applying shear forces by stirring at 3500 rpm (Ultra Turrax®, T-50, commercially available from IKA® Werke Staufen, Germany) for about 20 to 60 seconds. The particle size of the finely dispersed oil particles was measured using a Mastersizer® 3000 particle size analyzer (Malvern Instruments, 117 Flanders Road, Westborough, MA, USA).

The as-obtained preliminary oil-in-water emulsion was placed within an overhead mixer and agitated at 650 rpm while the remaining 0.75% of the PVA addition (1% PVA solution in deionized water) (Selvol™ 523, Sekisui Specialty Chemicals, Japan) were added as well as 0.7 g of the catalyst DABCO® (1,4-diazabicyclo[2.2.2]octane, Aldrich Chemical Corporation) and 60 g of a 10% maltodextrin DE 8 solution. The maltodextrin DE 8 solution was prepared by dissolving 6 g of maltodextrin having a dextrose equivalent (DE) of 8 in 54 g of deionized water. The resulting capsule slurry was cured at 70° C. for at least 30 minutes. Thereafter, additional 60 g of a 10% maltodextrin DE 8 solution were added, which was prepared in the same manner as previously described. The slurry was again cured at 70° C. for another 30 minutes. Finally, 0.60 g of Kelco-Vis™ DG (diutan gum, CP Kelco Inc.) were added while stirring the slurry at 1200 rpm for approximately 2 to 4 minutes before lowering the stirring speed to 850 rpm and raising the temperature to 80° C. for one more hour.

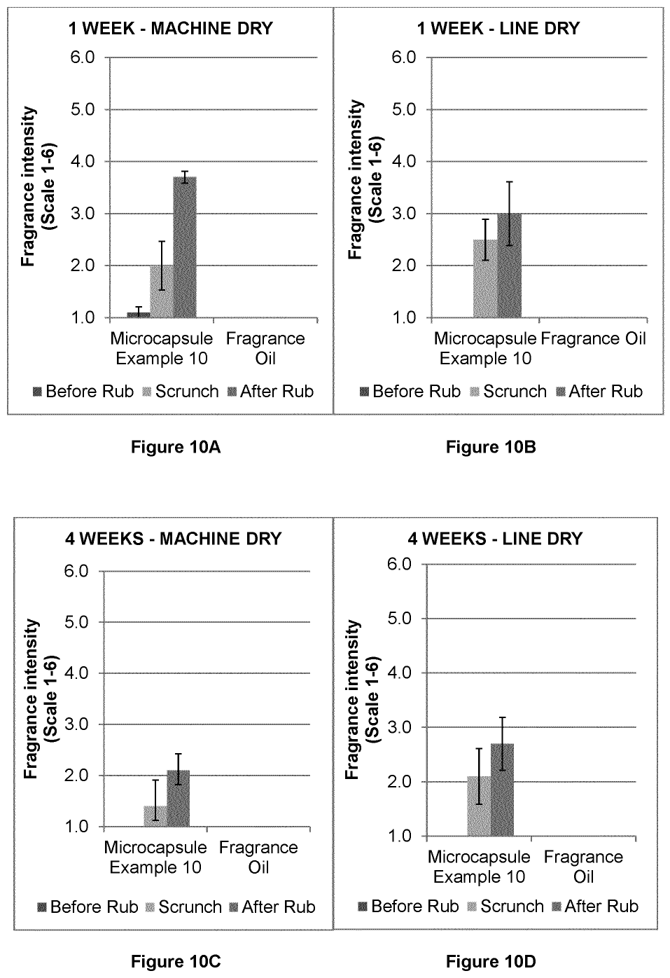

The as-obtained microcapsules have a sharp particle size distribution range within the aqueous phase indicating biobased core-shell microcapsules having uniform particle sizes. The core-shell microcapsules have a median particle size by volume (Dv(50)) of 13.7 μm (see FIG. 16J). Additionally, the core-shell microcapsules exhibit excellent release properties and show good performances and stabilities even after long-term storage in the product formulation or upon mechanical stress in the washing machine and heat (see FIGS. 10A to 10D). The combination of three different aliphatic polyisocyanates seems to positively influence the stability and performance.

### Example 11: Chito-Oligosaccharide and Glucosamine-Based Microcapsules II (90/10 HDI/MDI; Reduced Shell Wall)

Additionally, biobased microcapsules have been prepared showing a reduced shell wall). The shell wall amount is calculated based on the equivalent weights. In Examples 1 to 10 the relative shell wall amount/thickness is equivalent to that of the current state-of-the-art microcapsules. In Examples 11 and 12 the shell wall portion was reduced to almost the half in terms of weight or thickness compared to the state of the art. By virtue of the reduced isocyanate content, it was possible to considerably reduce the capsule thickness and achieve the formation of thinner shell walls.

Microcapsules according to the present invention were prepared using a chitosan oligosaccharide and glucosamine as the multi-functional nucleophiles and polyvinyl alcohol (PVA, Selvol™ 523, Sekisui Specialty Chemicals, Japan) as the capsule formation aid.

More specifically, 210 g of a fragrance material, here TomCap® (Symrise, Teterboro, NJ), were weighed out in a 250 ml-beaker and combined with 4.27 g of Desmodur® N-3400 isocyanate monomer (hexamethylene diisocyanate uretidione, HDI-uretidione; aliphatic polyisocyanate; equivalent weight: 193) and 0.48 g Mondur® M flakes (monomeric diphenylmethane-4,4′-diisocyanate; aromatic polyisocyanate; equivalent weight: 125.2), both from Covestro Corporation, to form an oil phase comprising the aliphatic and aromatic polyisocyanates in a relative weight ratio of 90:10 or a relative molar ratio of 85.4:14.6, respectively. Subsequently, 0.6 g of an oil soluble catalyst (bismuth neodecanoate, Aldrich Chemical Corporation) were added. In a separate 800 ml-beaker, a solution (302 g) containing 3.02 g of polyvinyl alcohol (PVA, Selvol™ 523, Sekisui Specialty Chemicals, Japan) in water was prepared to form the aqueous phase. The oil phase was then emulsified into the aqueous phase to obtain an oil-in-water fragrance emulsion having an average particle size ranging from 5 to 50 microns by applying shear forces (Ultra Turrax®, T-50, commercially available from IKA® Werke, Staufen, Germany) by stirring at 3500 rpm for about 20 to 60 seconds in the presence of 0.3 g of DABCO® (1,4-diazabicyclo[2.2.2]octane, Aldrich Chemical Corporation, St. Louis, MO, USA). The particle size of the finely dispersed oil particles was measured using a Mastersizer® 3000 particle size analyzer (Malvern Instruments, 117 Flanders Road, Westborough, MA, USA).

The as-obtained oil-in-water emulsion was placed within an overhead mixer and agitated at 600 rpm while 80 g of a solution mixture of 5% chitosan oligosaccharide (molecular weight <3000 Da) and 5% glucosamine (both from Biosynth Carbosynth®, United Kingdom) were added incrementally. The mixed chitosan oligosaccharide/glucosamine solution was prepared by dissolving 4 g chitosan oligosaccharide and 4 g glucosamine HCL in 72 g of deionized water. The pH-value was adjusted to approximately 10-11 by adding potassium hydroxide. The resulting capsule slurry was cured by heat at 70° C. for at least 3 hours. Finally, 0.60 g Kelco-Vis™ DG (diutan gum, CP Kelco Inc.) were added while stirring the slurry at 1200 rpm for approximately 2 to 4 minutes before lowering the stirring speed to 850 rpm and subsequent curing for one more hour.

The final core-shell microcapsules have a median particle size by volume (Dv(50)) of 22.6 μm within the slurry (see FIG. 16K). Although the capsule shell is significantly reduced, the microcapsules according to the invention show excellent release properties and stabilities, especially within the product formulation even after four weeks of aging, which are comparable to those of fully synthetic prior art microcapsules according to Comparative Example 13 (see FIGS. 11A and 11B). Although the as-prepared capsules exhibit considerably thinner shell walls compared to standard microcapsule shells the inventive microcapsules according to Example 11 (and Example 12) show excellent release properties and stabilities, even four weeks after aging in the corresponding product formulation. The capsule properties are comparable to those of capsules according to the state of the art having nearly double as thick capsule shells (such as those of Comparative Example 13).

### Example 12: Chito-Oligosaccharide and Glucosamine-Based Microcapsules III (86/14 HDI/MDI; Reduced Shell Wall)

Microcapsules according to the present invention were prepared using a chitosan oligosaccharide and glucosamine as the multi-functional nucleophiles and polyvinyl alcohol (PVA, Selvol™ 523, Sekisui Specialty Chemicals, Japan) as the capsule formation aid. Also, these capsules show a final capsule shell comprised of a reduced shell wall, which is corresponding to a considerable reduction compared to microcapsules according to the state of the art (e.g. according to Example 13 the shell is almost double in comparison to that of Examples 11 and 12).

More specifically, 210 g of a fragrance material, here TomCap® (Symrise, Teterboro, NJ), were weighed out in a 250 ml-beaker and combined with 4.09 g of Desmodur® N-3400 isocyanate monomer (hexamethylene diisocyanate uretidione, HDI-uretidione; aliphatic polyisocyanate; equivalent weight: 193) and 0.66 g Mondur® M flakes (monomeric diphenylmethane-4,4′-diisocyanate; aromatic polyisocyanate; equivalent weight: 125.2), both from Covestro Corporation, to form an oil phase comprising the aliphatic and aromatic polyisocyanates in a relative weight ratio of 86:14 or a relative molar ratio of 80.1:19.9, respectively. Subsequently, 0.6 g of an oil soluble catalyst (bismuth neodecanoate, Aldrich Chemical Corporation) were added. In a separate 800 ml-beaker, a solution (302 g) containing 3.02 g of polyvinyl alcohol (PVA, Selvol™ 523, Sekisui Specialty Chemicals, Japan) in water was prepared to form the aqueous phase. The oil phase was then emulsified into the aqueous phase to obtain an oil-in-water fragrance emulsion having an average particle size ranging from 5 to 50 microns by applying shear forces (Ultra Turrax®, T-50, commercially available from IKA® Werke, Staufen, Germany) by stirring at 3500 rpm for about 20 to 60 seconds in the presence of 0.3 g of DABCO® (1,4-diazabicyclo[2.2.2]octane, Aldrich Chemical Corporation, St. Louis, MO, USA). The particle size of the finely dispersed oil particles was measured using a Mastersizer® 3000 particle size analyzer (Malvern Instruments, 117 Flanders Road, Westborough, MA, USA).

The as-obtained oil-in-water emulsion was placed within an overhead mixer and agitated at 600 rpm while 80 g of a solution mixture of 5% chitosan oligosaccharide (molecular weight <3000 Da) and 5% glucosamine (both from Biosynth Carbosynth®, United Kingdom) were added incrementally. The mixed chitosan oligosaccharide/glucosamine solution was prepared by dissolving 4 g chitosan oligosaccharide and 4 g glucosamine HCL in 72 g of deionized water. The pH-value was adjusted to approximately 10-11 by adding potassium hydroxide. The resulting capsule slurry was cured by heat at 70° C. for at least 3 hours. Finally, 0.60 g Kelco-Vis™ DG (diutan gum, CP Kelco Inc.) were added while stirring the slurry at 1200 rpm for approximately 2 to 4 minutes before lowering the stirring speed to 850 rpm and subsequent curing for one more hour.

The final core-shell microcapsules have a median particle size by volume (Dv(50)) of 22.8 μm within the slurry (FIG. 16L). Although the capsule shell is significantly reduced, the microcapsules according to the invention show excellent release properties and mechanical and chemical stabilities, especially within the product formulation even after four weeks of aging, which are comparable to those of fully synthetic prior art microcapsules according to Comparative Example 13 (see FIGS. 11A and 11B).

### Example 13: Guanidine Carbonate-Based Microcapsules (Comparative Example; Polyurea-Based Microcapsules)

In a comparative example, microcapsules were prepared using guanidine carbonate as the multi-functional nucleophile and polyvinyl alcohol (PVA, Selvol™ 523, Sekisui Specialty Chemicals, Japan) as the capsule formation aid.

More specifically, 210 g of a fragrance material, here TomCap® (Symrise, Teterboro, NJ), were weighed out in a 250 ml-beaker and combined with 6.29 g of Desmodur® N-3400 isocyanate monomer (hexamethylene diisocyanate uretidione, HDI-uretidione; aliphatic polyisocyanate; equivalent weight: 193) and 1.57 g Mondur® M flakes (monomeric diphenylmethane-4,4′-diisocyanate; aromatic polyisocyanates; equivalent weight: 125.2), both from Covestro Corporation, to form an oil phase comprising the aliphatic and aromatic polyisocyanates in a relative weight ratio of 80:20 or a relative molar ratio of 72.3:27.7, respectively. In a separate 800 ml beaker, a solution (322 g) containing 1% of polyvinyl alcohol (PVA, Selvol™ 523, Sekisui Specialty Chemicals, Japan) in water was prepared to form the first aqueous phase. The oil phase was then emulsified into the aqueous phase to obtain an oil-in-water fragrance emulsion having an average particle size of 5 to 50 microns by applying shear forces (Ultra Turrax®, T-50, commercially available from IKA® Werke, Staufen, Germany) by stirring at 3500 rpm for about 20 to 60 seconds. The particle size of the finely dispersed oil particles was measured on a Mastersizer® 3000 particle size analyzer (Malvern Instruments, 117 Flanders Road, Westborough, MA, USA).

The as-obtained oil-in-water emulsion was placed within an overhead mixer and agitated at 600 rpm while 28 g of a 15% guanidine carbonate solution were added incrementally. The guanidine carbonate solution was prepared by dissolving 3.2 g of guanidine carbonate in 24 g of deionized water. The resulting capsule slurry was cured at 70° C. for at least two hours. Finally, 0.60 g of Kelco-Vis™ DG (diutan gum, CP Kelco Inc.) were added while stirring the slurry at 1200 rpm for approximately 2 to 4 minutes before lowering the stirring speed to 850 rpm and subsequent curing at 80° C. for an additional hour.

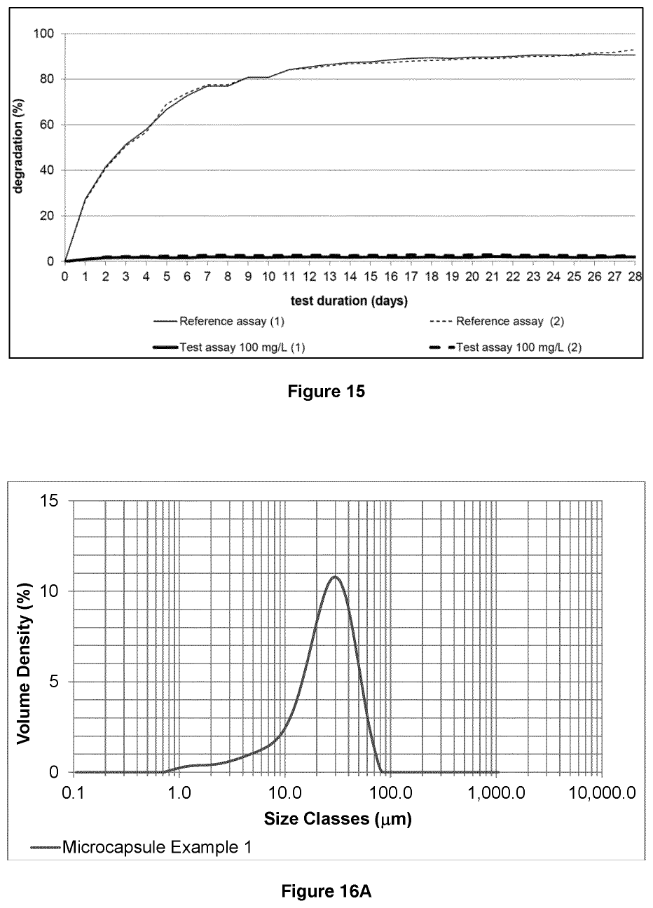

The final core-shell microcapsules have a median particle size by volume (Dv(50)) of 20.6 μm within the slurry (see FIG. 16M) and show no biodegradability (see FIG. 15).

### Sensory Evaluation of the Fragrance Core-Shell Microparticles

For the sensory evaluation, the microcapsules according to the invention were compared with microcapsules according to the state of the art (Comparative Example 13), i.e., microcapsule slurries produced as described above were compared:

0.3 g of the as-obtained core-shell microparticle slurries were dispersed in 30 g of a commercially available fabric softener (Downy® Ultra Free & Gentle™ Liquid Fabric Conditioner, Proctor & Gamble) and aged for one week or longer and preferably for one week, for two weeks and four weeks, respectively (marked as 1-week sample, 2-week sample etc. in the figures). In order to evaluate the sensory properties, each of the different fabric softener samples (each containing different microparticles) was added to 10 cotton-based face towels (small towels) and 10 cotton-based hand towels (larger towels). As reference, 0.1 g of the fragrance oil (TomCap®, Symrise, Teterboro, NJ) were directly dispersed in 30 g of the fabric softener.

The washing instructions were as follows: The towels (cotton cloth) were put into a washing machine and the fabric softener comprising the corresponding core-shell microcapsules or the pure non-encapsulated fragrance oil (reference) was added to the fabric softener compartment after aging for the time indicated above and the washing program started (device: combo (stacked) washing machine/dryer: Whirlpool® (USA); wash cycle: normal washing; water temperature: warm (32-44° C.); load size: medium).

After washing, the towels were divided into two groups and either air dried by hanging them out on a clothesline (indicated as “line dried” in the figures) or machine dried (indicated as “machine dried” in the figures) in the dryer. Subsequently, the towels were labeled correspondingly and stored in large plastic bags until testing.

The release of the fragrance was performed in three steps and the emitted fragrance intensity evaluated by 10 trained panelists. The intensity was determined based on a scale ranging from 1 (odorless) to 6 (very strong) in a blind evaluation. The first step describes the smelling of an untreated as-prepared towel. The second step describes the smelling of a lightly kneaded towel; for this purpose, the towel was subjected to slight mechanical stress by scrunching it. The third step describes the smelling after the towels were rubbed strongly by intensively moving them back and forth between the hands for several times, thus mechanically breaking the capsules and releasing the encapsulated fragrance oil. After each step, the intensity of the scent was evaluated by the panelists.

The microparticle formulations used for the comparison are summarized in Table 3 below.

TABLE 3
Analyzed core-shell microcapsule formulations.

| | Protec. | Shell material | | | | |
|---|---|---|---|---|---|---|
| | colloid | (Amino-) | Isocyanate- | | | |
| | (format. | saccharide - | Component (relative | Dv(10) | Dv(50) | Dv(90) |
| Preparation | aid) | Component | weight ratios) | [μm] | [μm] | [μm] |
| Example 1 | PVA | Glucosamine | Desmodur ® N-3400* | 8.0 | 25.3 | 47.7 |
| | | | Mondur ® M** | | | |
| | | | (80:20) | | | |
| Example 2 | PVA | Chitosan | Desmodur ® N-3400* | 7.7 | 22.9 | 42.2 |
| | | oligosaccharide | Mondur ® M** | | | |
| | | | (80:20) | | | |
| Example 3 | PVA | Chitosan | STABiO ™ D-370N* | 10.7 | 25.9 | 44.9 |
| | | oligosaccharide | Takenate ™ 600* | | | |
| | | | (50:50) | | | |
| Example 4 | PVA | Chitosan | STABiO ™ D-370N* | 10.4 | 22.6 | 38.0 |
| | | oligosaccharide/ | Takenate ™ 600* | | | |
| | | Glucosamine | (50:50) | | | |
| Example 5 | OSA | Glucosamine | Desmodur ® N-3400* | 17.7 | 32.1 | 53.8 |
| | starch | | Mondur ® M** | | | |
| | | | (80:20) | | | |
| Example 6 | PVA | Maltodextrin DE-8 | Desmodur ® N-3400* | 13.1 | 27.6 | 47.7 |
| | | | Mondur ® M** | | | |
| | | | (80:20) | | | |
| Example 7 | PVA | Maltodextrin DE-8 | Bayhydur ® 305* | 6.7 | 51.5 | 85.7 |
| | | | Takenate ™ 600* | | | |
| | | | (90:10) | | | |
| Example 8 | PVA | Maltodextrin DE-8 | Desmodur ® N-3400* | 11.6 | 26.4 | 45.8 |
| | | | Mondur ® M** | | | |
| | | | (80:20) | | | |
| Example 9A | PVA | Glucosamine | Desmodur ® N-3400* | 11.6 | 27.0 | 47.8 |
| | | Maltodextrin DE-8 | Mondur ® M** | | | |
| | | | (80:20) | | | |
| Example 10 | PVA | Maltodextrin DE-8 | Bayhydur ® 305* | 5.9 | 13.7 | 60.4 |
| | | | Takenate ™ 600* | | | |
| | | | STABiO ™ D-370N* | | | |
| | | | (50:30:20) | | | |
| Example 11 | PVA | Chitosan | Desmodur ® N-3400* | 6.86 | 20.4 | 38.7 |
| (reduced | | oligosaccharide/ | Mondur ® M** | | | |
| shell) | | Glucosamine | (90:10) | | | |
| Example 12 | PVA | Chitosan | Desmodur ® N-3400* | 7.97 | 22.8 | 40.3 |
| (reduced | | oligosaccharide/ | Mondur ® M** | | | |
| shell) | | Glucosamine | (86:14) | | | |
| | Protec. | Amine- | Isocyanate- | Dv(10) | Dv(50) | Dv(90) |
| Preparation | colloid | Component | Component | [μm] | [μm] | [μm] |
| Comparative | PVA | Guanidine | Desmodur ® N-3400* | 8.5 | 20.6 | 36.7 |
| Example 13 | | Carbonate | Mondur ® M** | | | |
| | | | (80:20) | | | |

*aliphatic
**aromatic

The microparticle formulations prepared according to Examples 1 and 6 (FIGS. 1A/1B, and FIGS. 6A/6B) were compared to standard polyurea microcapsules prepared according to Comparative Example 13.

The performance of the inventive biobased (amino-)saccharide-based microcapsules using saccharides and/or aminosaccharides having less than 20 monomeric units as building blocks is comparable to and even slightly better than the performance of the fully synthetical guanidine crosslinked polyurea-based microcapsules according to the state of the art after machine drying. However, for linen dried samples significantly higher fragrance intensities have been evaluated for samples treated with the fabric softener comprising the inventive biobased (amino-) saccharide-based microcapsules. Mechanical stress or heat seemed to increase the release of volatiles from the clothes treated with the emulsion. The lower fragrance intensities for towel samples dried in a dryer can be explained based on an increased breaking of the microcapsules due to the mechanical impact within the dryer and increased evaporation due to heat.

However, the inventive biobased microcapsules seem to be more stable compared to the fully synthetical state-of-the-art microcapsules and are able to suppress an evaporation of the volatile fragrance components more efficiently. The high increase in intensity after scrunching and/or rubbing (which is considerably higher compared to the comparative capsules) indicates an efficient encapsulation of the fragrance material. This is particularly evident based on FIGS. 6A and 6B. The experiments indicate a high mechanical and thermal stability of the inventive microcapsules (washing machine, drier) while still allowing for a targeted release of the active ingredient. Additionally, the inventive microcapsules show increased chemical stabilities, e.g., when incorporated and aged within consumer product formulations allowing for an efficient encapsulation of the active ingredient and effectively maintaining the long-term product quality. Therefore, the inventive capsules allow for an efficient encapsulation and an excellent and targeted release of the active ingredients. In addition, the inventive microcapsules are less affected by the drying process within the dryer than the fully synthetical state-of-the-art microcapsules. Consequently, the inventive biobased core-shell microcapsules are highly suitable for the incorporation of a variety of consumer product formulations.

Moreover, samples of the fabric softener containing 0.3% by weight of the respective inventive fragrance microcapsule slurries according to Examples 1 to 12 containing 33% by weight of the active fragrance oil (corresponding to 0.1 wt.-% pure fragrance oil) were compared to 0.1 wt.-% fragrance oil directly incorporated into the fabric softener as reference and compared for both, the machine dried and line dried towels (FIGS. 1 to 12). While the free non-encapsulated fragrance oil quickly evaporated and was not perceivable after the drying process and storage, the biobased microcapsules according to the invention enabled a stable encapsulation of the fragrance material and showed an excellent and targeted release behavior and, as a result, allowed for a highly perceptible fragrance intensity (i.e., high sensory performance).

Even after four weeks of aging in the softener an efficient encapsulation of the fragrance material could be observed, indicating a high stability of the inventive core-shell microcapsules within the softener and thus within consumer product formulations (see for example FIGS. 8A to 8D; FIGS. 10A to 10D; FIGS. 11A and 11B; FIGS. 12A to 12D).

Additionally, microcapsules according to the present invention having reduced shell thicknesses (see Examples 11 and 12) show excellent performances and stabilities comparable to those of state-of-the-art microcapsules having considerably thicker shell walls.

Consequently, it can be concluded that the biobased core-shell microcapsules according to the invention are more stable towards mechanical impact and heat while simultaneously allowing for a targeted release of the encapsulated active material (see machine dried samples). Moreover, the inventive biobased microcapsules efficiently encapsulate the active material for more than four weeks in consumer product formulations while the state-of-the-art capsules show considerable losses in active material with time (see line dried samples). Therefore, it can be concluded, that the biobased microcapsules according to the invention exhibit an improved balance of efficient encapsulation, stability, and targeted release properties/performance.

### Evaluation of the Biodegradation of the Inventive Core-Shell Microparticles

The biodegradability of the inventive core-shell microcapsules was determined as follows:

The biodegradation of the microcapsule slurry in the environment involves the biological breakdown of the polymeric shell as per the invention. Measurement of biological activity or decomposition of the shell material could be determined under different environments in soil, ocean water, or sludge notably under OECD guidelines.

The OECD tests which can be used to determine the ready biodegradability of organic chemicals include the six test methods described in the OECD Test Guidelines No. 301 A-F: DOC Die-Away Test (TG 301A), CO₂ Evolution Test (TG 301B), Modified MITI Test (I) (TG 301C), Closed Bottle Test (TG 301D), Modified OECD Screening Test (TG 301E) and Manometric Respirometry Test (TG 301F). The following pass levels of biodegradation, obtained within 28 days, may be regarded as evidence of ready biodegradability: 70% DOC removal (TG 301A and TG 301E); 60% theoretical carbon dioxide (ThCO₂) (TG 301B); 60% theoretical oxygen demand (ThOD) (TG 301C, TG 301D and TG 301F). More details can be found in the official OECD Guideline for the testing of chemicals: OECD (2006), Revised Introduction to the OECD Guidelines for Testing of Chemicals, Section 3, OECD Guidelines for the Testing of Chemicals, Section 3, OECD Publishing, Paris.

### Test System:

The test method and test system (inoculum) used for the analysis of the biodegradability of the microcapsules according to the invention are consistent with the standardized testing guideline and procedure according to the OECD 301F biodegradability test.

### Isolation of the Shell Material (Sample Preparation):

The shell wall comprising 1% to 10% by weight of the total core-shell microcapsule (minus water) was obtained by extraction of the payload (i.e., the core) and removal of any water-soluble reagents (protective colloid, unreacted (amino)saccharides and salts) by washing.

The isolation of the shell was accomplished by a multi-step process comprising the following steps:
1) Washing with water to remove any soluble materials followed by shell isolation via centrifugation.
2) Solvent extraction of the hydrophobic “core” using organic solvents such as lower alcohols, acetone and the like.
3) Heat drying the as-obtained shell material to remove any remaining solvents and water followed by grinding to obtain shell powder, i.e., the shell material in powdered form.

The isolation of the shell wall could also be accomplished using other processes such freeze-, vacuum- or spray drying or a more elaborated process such as supercritical CO₂ extraction.

The biodegradation study was performed on the as obtained isolated microcapsule shell powders and submitted for test under the standardized OECD 301F procedure. In a first biodegradability study the microcapsule shell material according to Example 1 has been analyzed under the standardized OECD 301F procedure (Sample #1) for its ready biodegradability in a manometric respirometry test according to the OECD Guideline for Testing of Chemicals, No. 301F (1992). In accordance with the test guideline the test was prolonged to 45 days since the biodegradation curve of the test item showed that biodegradation had started but the plateau had not been reached by exposure day 28. It was found that Sample #1 showed inherent, primary biodegradability. In addition, the test item had no inhibitory effect on the activity of activated sludge microorganisms at the tested concentration of 100 mg/L. However, a second sample (Sample #2) of the microcapsule shell material according to Example 1 indicated that the ready biodegradability was almost reached (see FIGS. 13 and 14).

An analysis of a comparative test sample corresponding to a capsule shell prepared according to Comparative Example 13 and corresponding to the capsules disclosed in U.S. Pat. No. 6,586,107 B2 shows that these fully synthetical guanidine-based capsule materials are not biodegradable at all (see FIG. 15). Therefore, based on the degradation curves, it can be concluded that the core-shell microcapsules according to the invention are more biodegradable than the currently available state-of-the-art microcapsules and thus more suitable in terms of more eco-friendly consumer products.

### Consumer Product Formulations

In the following a variety of suitable consumer product formulations according to the invention is shown comprising the inventive core-shell microcapsules allowing for an efficient encapsulation and excellent release of the active ingredient.

TABLE 4
Cleaner, APC liquid, alkaline pH 8-10 (amounts in wt.-%)

| Ingredients (INCI) | Amount |
|---|---|
| Aqua | 59.06 |
| Tri Sodium Citrate Dihydrate | 3.00 |
| Sodium Laureth Sulfate | 30.00 |
| Trideceth-9 | 5.00 |
| Ethanol | 2.00 |
| Citric Acid 10% solution | 0.24 |
| 1,2-Pentanediol (Hydrolite ® 5) | 0.50 |
| Mixture of 5-Chloro-2-methyl-2H-isothiazol-3-one and | 0.10 |
| 2-Methyl-2H-isothiazol-3-one | |
| Microcapsules according to Example 1-12 | 0.30 |

TABLE 5
Fabric softener (amounts in wt.-%)

| Ingredients (INCI) | Amount |
|---|---|
| Aqua | 72.10 |
| Dialkylester ammomium methosulfate | 16.60 |
| Polydimethylsiloxane | 0.30 |
| Magnesiumchloride | 10.00 |
| 1,2-Pentanediol (Hydrolite ® 5) | 0.60 |
| Mixture of 5-Chloro-2-methyl-2H-isothiazol-3-one and | 0.10 |
| 2-Methyl-2H-isothiazol-3-one | |
| Microcapsules according to Examples 1-12 | 0.40 |

TABLE 6
Liquid detergent (amounts in wt.-%)

| Ingredients | Amount |
|---|---|
| Deionized water | 39.60 |
| Optical brightener | 0.10 |
| Coconut fatty acids (C₁₂-C₁₈) | 7.50 |
| Potassium hydroxide 50% solution | 4.30 |
| Propane-1,2-diol | 5.00 |
| Fatty alcohols C₁₂-C₁₅, 8 EO | 12.00 |
| Na-salt of secondary alkyl sulfonates (C₁₃-C₁₇) | 17.00 |
| Triethanolamine | 2.00 |
| Trisodium citrate dihydrate | 5.00 |
| Dequest ® 2066 (Diethylenetriamine penta(methylene | 3.00 |
| phosphonic acid)) | |
| Ethanol | 3.00 |
| Enzymes | 0.70 |
| 1,2-Pentanediol (Hydrolite ® 5) | 0.50 |
| Microcapsules according to Examples 1-12 | 1.00 |

TABLE 7
Liquid detergent concentrate (amounts in wt.-%)

| Ingredients | Amount |
|---|---|
| Deionized water | 12.9 |
| Coconut fatty acids (C₁₂-C₁₈) | 10.0 |
| Fatty alcohols C₁₂-C₁₅, 8 EO | 26.0 |
| Na-salt of secondary alkyl sulfonates (C₁₃-C₁₇) | 26.5 |
| Triethanol amine | 8.5 |
| Na-salt of fatty alcohol sulfates C₁₂-C₁₄ | 3.0 |
| Ethanol | 5.5 |
| Urea | 4.5 |
| Enzymes | 0.9 |
| Citric acid | 1.0 |
| 1,2-Pentanediol (Hydrolite ® 5) | 0.7 |
| Microcapsules according to Examples 1-12 | 0.8 |

TABLE 8
Toilet cleaner (amounts in wt.-%)

| Ingredients | Amount |
|---|---|
| Water | 93.0 |
| Kelzan ® ASX-T | 0.5 |
| Parafin sulfonate, sodium salt | 1.0 |
| Citric acid | 5.0 |
| Colorant (FD & C Yellow No. 6) | 0.1 |
| 1,2-Pentanediol (Hydrolite ® 5) | 0.3 |
| Preservative (Benzisothiazolinone, Glutaral) | 0.05 |
| Microcapsules according to Examples 1-12 | 0.6 |

TABLE 9
Dish washing concentrate (amounts in wt.-%)

| Ingredients | Amount |
|---|---|
| Sodium laurylsulfate | 31.0 |
| Propane-1,2-diole | 6.0 |
| Ethyl alcohol 96% | 7.0 |
| Palm tree glucosides | 6.0 |
| Coco betaine | 18.0 |
| 1,2-Pentanediol (Hydrolite ® 5) | 0.4 |
| Microcapsules according to Examples 1-12 | 0.5 |
| Water | 31.6 |

TABLE 10
Dish washing concentrate (amounts in wt.-%)

| Ingredients | Amount |
|---|---|
| Palm tree glucosides | 4.0 |
| Sodium lauryl sulfate | 45.0 |
| Coco betaine | 8.0 |
| Ethyl alcohol 96% | 1.0 |
| Colorant (C.I. Pigment Blue 15) | 0.05 |
| 1,2-Pentanediol (Hydrolite ® 5) | 0.2 |
| Microcapsules according to Examples 1-12 | 0.7 |
| Water | Ad 100 |

TABLE 11
Solution for wet wipes (amounts in wt.-%)

| Ingredients | INCI | Amount |
|---|---|---|
| SymSol ® PF-3 | Water (Aqua), Pentylene Glycol, | 2.00 |
| | Sodium Lauryl Sulfoacetate, | |
| | Sodium Oleoyl Sarcosinate, | |
| | Sodium Chloride, Disodium | |
| | Sulfoacetate, Sodium Oleate, | |
| | Sodium Sulfate | |
| Dragosantol ® 100 | Bisabolol | 0.10 |
| Glycerol 99.5 P. | Glycerol | 5.00 |
| Water | Water (Aqua) | Ad 100 |
| Hydrolite ® 5 | 1,2-Pentanediol | 5.00 |
| D-Panthenol 75 W | Panthenol | 0.80 |
| DragoCalm ® | Water (Aqua), Glycerol, Avena | 1.00 |
| | Sativa (Oat) Kernel Extract | |
| Witch Hazel-Distillate | Hamamelis Virginiana (Witch Hazel) | 1.00 |
| | Water, Water (Aqua), Alcohol | |
| Allplant Essence ® Org. | Pelargonium Graveolens | 1.00 |
| Rose Geranium P | Flower/Leaf/Stem Water | |
| Preservative | Phenoxyethanol | 0.30 |
| Microcapsules according | | 0.50 |
| to Examples 1-12 | | |

1. A core-shell microcapsule, wherein the shell comprises a polymeric material being a reaction product of at least one polyisocyanate having at least two isocyanate groups and at least one saccharide and/or at least one aminosaccharide having less than 20 monomeric units and wherein the core comprises at least one active ingredient.

2. A core-shell microcapsule according to claim 1, wherein the saccharide(s) and/or aminosaccharide(s) are bio-derived.

3. A core-shell microcapsule according to claim 1, wherein at least one of the one or more polyisocyanates having at least two isocyanate groups comprises an aliphatic structure.

4. A core-shell microcapsule according to claim 1, wherein the at least one saccharide and/or at least one aminosaccharide has 15 or less monomeric units, and preferably 10 or less monomeric units.

5. A core-shell microcapsule according to claim 1, wherein the at least one saccharide and/or at least one aminosaccharide having less than 20 monomeric units is selected from the group consisting of: monosaccharides monomeric aminosaccharides, disaccharides, dimeric aminosaccharides, linear and/or branched oligosaccharides, linear and/or branched oligomeric aminosaccharides, and mixtures thereof.

6. A core-shell microcapsule according to claim 1, wherein the at least one saccharide and/or at least one aminosaccharide has at least two functional groups independently selected from the group consisting of primary and secondary hydroxyl groups (—OH) and primary and secondary amine groups (—NH₂, —NH—).

7. A core-shell microcapsule according to claim 1, wherein the at least one active ingredient is selected from the group consisting of: cosmetic active ingredients, agrochemicals, perfume substances, perfume oils, aroma substances, aromas, active pharmaceutical ingredients, dyes, UV-active substances, optical brighteners, bodying agents, drape and form control agents, smoothness agents, static control agents, wrinkle control agents, sanitizing agents, disinfecting agents, germ control agents, mold control agents, mildew control agents, antiviral agents, antimicrobials, drying agents, stain resistance agents, soil release agents, malodor control agents, fabric freshening agents, dye fixatives, color maintenance agents, color restoring/rejuvenating agents, anti-fading agents, anti-abrasion agents, wear resistance agents, fabric integrity agents, anti-wear agents, rinsing aids, UV protection agents, sun fade inhibitors, insect repellents, anti-allergenic agents, flame retardants, water-proofing agents, fabric softening agents, shrinkage resistance agents and/or stretch resistance agents, fluorescent paints, solvents, waxes, silicone oils, lubricants, cooling agents, TRPV modulators, and mixtures of the above-mentioned active ingredients.

8. A core-shell microcapsule according to claim 1, wherein the microcapsule has a median particle size by volume (Dv(50)) of 1 μm to 100 μm.

9. A core-shell microcapsule according to claim 1, wherein the microcapsule shell is inherently biodegradable according to the OECD 301F biodegradability standard.

10. A microcapsule slurry comprising a plurality of core-shell microcapsules according to claim 1 dispersed in an aqueous phase, wherein the microcapsule concentration within the aqueous phase is greater than 5% and less than 70% by weight.

11. A process for preparing a microcapsule slurry, comprising the following steps:
(a) providing an oil phase comprising at least one polyisocyanate having at least two isocyanate groups and one or more active ingredients;
(b) providing a first aqueous phase comprising at least one formation aid;
(c) providing a second aqueous phase comprising at least one saccharide and/or at least one aminosaccharide having less than 20 monomeric units, optionally wherein the second aqueous phase further comprises one or more additional formation aids;
(d) blending the oil phase and the first aqueous phase to obtain a preliminary oil-in-water emulsion;
(e) blending the second aqueous phase and the preliminary emulsion obtained in step (d) to obtain a microcapsule slurry; and
(f) curing the microcapsules obtained in step (e).

12. A process for preparing core-shell microcapsules comprising preparing a slurry according to the process of claim 11, and subsequently comprising the step of:
(g) isolating the core-shell microcapsules from the slurry.

13. The process for preparing a slurry comprising core-shell microcapsules according to claim 11, wherein the formation aid(s) is/are surface-active agent(s) and/or colloidal protective agent(s).

14. The process for preparing a slurry comprising core-shell microcapsules according to claim 11, wherein the molar ratio of the at least one polyisocyanate having at least two isocyanate groups to the at least one saccharide and/or at least one aminosaccharide having less than 20 monomeric units is in the range of from 1:3 to 1:1.

15. The process for preparing slurry comprising core-shell microcapsules according to claim 11, wherein the formation aid(s) is/are biopolymeric derivative(s).

16. A slurry comprising core-shell microcapsules obtained by the process according to claim 11.

17. A core-shell microcapsule obtained by the process according to claim 12.

18. A consumer product comprising the core-shell microcapsules according to claim 1, wherein the product is selected from the group consisting of: cosmetics, personal care products, shampoos, rinse-off conditioners, deodorants, antiperspirants, body lotions, textile care products, homecare/household products, liquid detergents, all-purpose cleaners, laundry and cleaning agents, fabric softeners, scent boosters, fragrance enhancers and pharmaceuticals.

19. The core-shell microcapsule according to claim 4, wherein the at least one saccharide and/or at least one aminosaccharide has 10 or less monomeric units.

20. The core-shell microcapsule according to claim 8, wherein the microcapsule has a median particle size by volume (Dv(50)) of 5 μm to 50 μm.
